SEQUENCE LISTING (4 sequences)

SEQ ID NO: 1 - DNA, 26419 bp, Homo sapiens
  allele (1272)..(1272): PS2 polymorphic base adenine or guanine
  allele (1702)..(1702): PS6 polymorphic base cytosine or thymine
  allele (1981)..(1981): PS11 polymorphic base cytosine or thymine
  unsure (3055)..(3155): N′s represent unknown nucleotides
  allele (9682)..(9682): PS13 polymorphic base guanine or adenine
  unsure (10786)..(10886): N′s represent unknown nucleotides
  allele (12027)..(12027): PS15 polymorphic base guanine or adenine
  allele (16448)..(16448): PS21 polymorphic base guanine or adenine
  unsure (17114)..(17214): N′s represent unknown nucleotides
  allele (25065)..(25065): PS26 polymorphic base adenine or guanine
  allele (25139)..(25139): PS27 polymorphic base cytosine or thymine
  allele (25215)..(25215): PS28 polymorphic base cytosine or thymine
cactaatatttagaggttaatttatcacagacgcaagagtttccataactaatgcatccaggggttccctgtctatgattttagacaatacctgacaaagcagtgagtaacttttaatcatttactgagaaagttgttccttttgcaatgctgtggcgcccatgagagagtggatagaaagaggtagcttgtgttttctaaacaacaaatactttgaatacacatacaaagaagaaatacaaatggccaacaagcacatgaattgatgcttaacatcatttctcattcaggaagtacaaatccaaaccacaatgagatatcacttcacacccactaggatggaacaaaacaaaaacagaaaataacaagtgttggcgatgatgtggggaaattggaaccctttcattgttggcagaaatgtaaatggtcacagctgctatggaaatcagttggttagtccctcaaaaagttaaacatagaattattgtatgacccagcaattgcactgcctgagtacatacctgaaagaattgaaaacaggcacccaaacatatatttgtacaagaatgttcacagtagcattactcacaaaagtcaaaaagtagatatagtagcccactcaaatgtccatcattagatgaacagataaagaaaatgtggtatatacaggtacatacacaaaatattactcaacaataagaagaaatggtgctataatggggatgaacctgaaaaacattgcgctgagtcaaagaagccattcgctaaagaccacacattgtatgggagccaaggcagtatccagcgagactctggcctctggctgggctgagtgacttcttttcccccagtgcctttgaactctctgcacccaactcagccatgagcaatgctgtcaccagcaatgggcagaggggctggggcaagagtgggatggaaagaatactggatgtggagaccaaatgtgacagtgtccttgcccggctgcctgctttagatctcaggggacacgccatttcctctccgataaactctaagctcttagggtaacccatggacctgcctggctgggtctttgttgacatttacaaggagcctctcccgcagccctccaaggggtccctctcccctgcaggctgcatgcccagttctccctggacctccccaaccccctctctactaacccccgtttgaacaggaagacacagcctgagccaggtctcccctcacaaatctccccaccagggctggatcgcgcaacaaatagagccrgccccagggcactcgccggcagggcgcagccggtgatcgtcacagtccttcgaaattcttaagatctaggtcttgctgcacccccacaacctacaaacagcgtcggggccttctctgcacctccagttcccagctcacctccctcagtgtcacagccggttacctttccttcctccctggcaagggagggcaagacttggggcttgctgactccaggcccagcccagcccggggcacccaggagcccctcaattgctactcaaacaagacaagaagcggcccgagttagtggccagctccaccatgcactacacatcctgacctctctgagcctctactgtcactcggggtcacaaccctttcctgagcacctcccggggcagggggcgatgacacacatgcagctgcctgggggaggccggcggtgtcycctcctttctggaaagcggagggtcctggtgggctctggaaacgcagcccagacctttgcaatgctaggaggatgagggcggagacctcgcggtccccaacaccagactcccgccgccaccgcgcccggtcccgccctccccactgcccccccagctccccgacccaggcgccccgcccggccagctcctcacccaccccagccgcgactgtctccgccgagcccccggggccaggtgtcccgggcgcgccacgatgcggccgcggctgtggctcctcytggccgcgcagctgacaggtaaggcggcggcgcgcgggctacccaagggtctgcgctcccggggcctgagcggggaggtgataagtggctgtcctggccctggtcctggcagggtgcagcgtcgagcccgcggtggcggggcgcccgggaggcagcttggcaggcacggtccctaagggtggaaataaaatacccccatatcgcattaccccgggggaccggagagcccctggactgaggccacctcccctcaaaagcctggacgcaggagaaggggaggcagtgaaaaggggagcgagtgagggaaggaaagagagggtcgctggaggtcaccaggggaaggaaacaggtccctgcccagggtccccgcaggatgtgctcggaggaaggttggccaggccatgggtcctgtggacacatttttattacttccggggaagtgtttgtagtacaatcagacaaacatcgggcgttctcagttctcggagggctagggcagggtgatccctctggctcccgttctccctgatgtcgctggtgttgggtgtcatgggtagcgtggctccctaggaagcagcctggaggttgggttgttggagttggatggggcatctggattgtccttctgaataagcccagccagactgccttttggaagaagcctccacacacccggaaaactacccaaactggtcgatttgctctcgtgcccaaaaagcccaaagctggtcatcttgccagggggtgacaaggcctgcccgccgggggttctggcccagtgagtgcaacatcataaatagaaacagcccaggccgttcagagcactaaagtcattactgatggatctagaaacatcaggtccctctgagaatggaattcctgggttaccgaggggaacatgcagtgaggcctggctccatgtccagcacacagcagggccttggccacagcccggagtattcagttcctaggggacaaaaagcaagtgggccccgggaagagccggggtgtgtctcagccaggagaggaaatgcacactcacagcccaggccttgctcttacaggagtcgcttgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngttctgatccttctccttgagatttaatttacctccagtaacaaccctgtgatatgagacacagggacagtatttacctgtgtacatggccatacagtttacagtgtgcttttttggacagcatttcatttatttccaaccctcagaggcaggcaggacaacagccccaagctgtcctccgttcataagaagacagaggctcggtgactggcctgaggtctcacagctggcacggccagaggctgggttcagggccaggtctgtgagtcctgctctagctcctcatgaataagcccctgcttgccgggtgcttctcgtcagagtctaagatgaactcccagccctgcttctgtggtgtctgtccttccactggggaatagggatccagtgacccatcagttgctgggctcgggggtcatgggctgtaattgtacctaccagggtctgtccagcaacggacagaccagccacatgctctgctgtgattctgggctcctctcccagcctgtcctgggtctccttcggcagtttggagcctaccgaagggctgtacatccagtggcagccaggggaggtgaaaggcaaccagctttggcttcagacttcaaatccttgagtttgactcctaggccacttacatactgtaagtgggactttgaacaagtaacttcatgcctcagtttcctcatctgtgtaatgaggattaaaaatcctggatctatccttttgtggtggtacatgcctatactctcagctacttgggaggctgaggtgggaagatagattgagcccaggaggtcaaggctgaagtgtactatgatagtgcctgtgagtagccactccactccaacctgggcaacataacaagaccccatgtcttttaaaaaattccaaagccagtttgaggcttgattgagttcctttatgcaagggccaggcatataagtacacaataaacaaatggcagctctctcccctgtacctttgtaaagctgaacttagcaaacacctcccttccagatgatctcctgggaggtggggtatgtgaactctgagtccaggagccaacagctgcgctcaacacactccctggacccagtaactgggcaggtccttccatggcttttcctttgacacttgactcagtgtgactgcaaggtcccacagctgtggctaggaaaatgtgtgatgtctctgttcttggcttttcctagttctccatggcaactcagtcctccagcagacccctgcatacataaaggtgcaaaccaacaagatggtgatgctgtcctgcgaggctaaaatctccctcagtaacatgcgcatctactggctgagacagcgccaggcaccgagcagtgacagtcaccacgagttcctggccctctgggattccgcaaaagggactatccacggtgaagaggtggaacaggagaagatagctgtgtttcgggatgcaagccggttcattctcaatctcacaagcgtgaagccggaagacagtggcatctacttctgcatgatcgtcgggagccccgagctgaccttcgggaagggaactcagctgagtgtgggtaagaagcaggctcaatgctatcagtgagcaccgattgtgtgccaggcacgtgccaggcactgggaccaccaggcttcagtgtgccccactctgcagccgtgactaatggcactgctgctaatagtaacagaaagaacatgtggagacttacccaggccaagcccctccaagtgtttacatagttagcttgcttaatgctgccaaccaccctgaaggtaggtctattacctaaccactttaggtgggaataagatgaaactagaagagacgggccagagtcagagatgagcattgtgcttctcttttttatttatttattattattattttttttgagacagagcctcactgtgttgcccaggctggggtgcagtggtgcgatcttggctcactgcagcctctgcttcccgggttcaagtgattctcctgcctcagcttcccgagtagctgggattacaggcatgagccaccacacctggctaattttttgtatttttagtggagacgagatttcaccatgttggccaggtgggtcttgaactcctgacctcaagtgatccacccaccttagcctcccaaagcgctaggattacaggcgtgagccaccatgcctggccaggcattattcttcttatggggacagggcagtgctgtccttcaggccacatcagggacattcaaagctacggctggattgggctgagccccttggccttatagtggtgttagaaatccccagagtgtgcacacagggaaaggaggccctcactgccctgatatacccagtgagggcatttctaggggcaggtgcccttatgttctctgtgagggcctacagctcgaattcatacctgagtcgaagttagtaaggagactgagccaggcccagctctgcttgcaaacttgccatgctttctcccctgggcctcagtgttctcttctgtgtcacacgggggtgctgatggcctccatgacccacttaatcccagagaagctgcagacctctgacttaagtcagcaagtccagcagacgcacatttcaagccacagatgaaattctaaatttttaaagtcttgtttaaaaaagcaaaaagaagcaagtaaaattaatttaaataatgttatatttaactcaatttattaactatattttcatctcaacatgtaatcaatataaaaagtattgctgaaatattttacattcatttttcatactaagtgttcgatgagcattttacataaaacatatggctcatctcgattcagactggccacatttcatgtgctcagcagcacctgtggcatattggacagaaggcatttagttatgggtggtaactctcacagcctggctctggctctagccccacctccagcttggcccaaggctggagccaaccccctccagggctcccactgccctgcttcctgctcacttgagggcactaaaagccacaaggggacccagaaattagaccactcagcgtgaggaagtgcaggggcagctggggacagcagcacaattaggaagacacaaaccaaagtgatgccccgccacctcttgctagcctgggagggtgtattgcaggcaagaagctgaggcatcccttcacgtgggggtccactgattttcgtattgaaaaatggactttgggagtttggggtttggggtttggggtttggntttagcttattgtttttgtctctgtaagccccacacctttccactcatgagatttcagagtctgagaaggtggagggtacaataaacacagctagtttccagagcttcccaccgcctccatcccaaccgcactcactcctgacttccgtgtttccaaaccccaaacccctcccagcactataggaatgtcaatgcctctctccagctctggcttgcagccaaacctgcttctccccagggcttgcaaccagaccttcttcggagccgaaccccaccttctcacccaagaaaggccttttagaggacaaagctgggatggttgagaaagtgtttggggaaagacagggcctttttcaggcccttaagtatatcatgggctcgaagttttattccttttaagaacagagagggagagattaaactgggtgctcaggaggaatccagggggacctggctctaggcaagaagaatttaaaacagaacagtcccccaacctgagtctcccactagacccaggcggcggtgcccagccaaggcactgagggcccaaagagttggctcccctctgggctcaccactgaccatctgaaagacgtccctgcttcctcgggtagaaaataagtcaaacagaactacttctagggccatgaggattgaaggaggcaacactcccaggagagggccaggctcgttgcacgctctccgaccctgctgatgacagtttcctcttacaatagttgccctcagcactatgatgacccagggccttgggtggacctaaagtgttcaacttttgtcagaaaatctttgcacgtgaggtagtgtggcagaacgagaagcacaggttctagaatcaaggtcttttggtgcagatactggctctgcagccagtggctgtctaactttggacacattatttcagctctctgagcctaggcttcctcctggataaaatggggacaacagtgcctgccccaaaagttgttgtgtccagtaaacttatgctgaaaggctcagcactgctccccacacatggttggagcttagtgcactttcatcctagttgcttcttcccatcaatgtcttgaagcagacaaacagatcgtgttctctccgctttccagaggaggctcagagaggtgaggggttaaaatgtctacttttatatagaagatgccagcccaaaaatgactgatctgtgggcttcccctgcctgttcccccttacaaccccccaaaaccttttttttttttgagacggagtcactgtcacccaggctggagtttagtggcaggatcttggctgactgcaacctctgcctcacaggttcaaacgactctgctgcctcagcctcctgagtagctgggattacaggcacgtgccaccacgcccaactaatttttgtatttttagtaggcacagggttttgccatgttggccaggctggtcttggactcctgacctcaggtgatctgcctgcctcggcctcccaaagtgctgggattacaggcgtgagtcaccgcgcccagccttaatatttcttttcttttcttttttttttttctgtccccctgactctagagtttaggttcatggtgtctccttactccctctcctaccattttggagaaaatcagtgcagagagatctgtttccttaacccacaaaggcaagggttggactctgtggccagcagtgcccccttcatccccagtaggcatcagagccacagctgtcttcctttgctctgggtgctggcccactgcccctcccaacacttcagcaggggtcactgggtaaaaatgaaggagaggggtgacccatccaggagtcaggacaggggtttggggtcccaacacagcactcaccacttggggctgcaggggccagctcccctcatctctggcgttcacctttgaaattaaagtttgagactgaacttcccttagctctggtattatatatatattttagggccatctgagaaggaaaaccaatccccaaatatgacacctgtgtggtactctacatgcagaaaagggcaccccctccttccatgagcttcctccatgtgcttagggaatgccgagttgggcggaggggctcgttgtgccaaaggctgggcgatccagccctggggctcaaggtcttcagccaggtggagccacatctactgaaactttttttggacacactgtccctttcattctcagataatcccaagaaggaggtggcagttatcattatttcagcagataaggaaatcaaggcacagaaaaatgctatgacttgtcccaagtcacacagcggttaatcaggggtgtaggtcggccgggtgcggtggctcgtacctgtaatactagcacttttggaggccgaggtgagtggattacctgagatcaggagttcaagaccagcctggccaacatggtgaaaccccgtctctactaaaaatacaaaaaattagacgaatgcagtggcacgacgaatgcagtggcacgacgaatgcagtggtacgtgcctgtaatcccagctactctggaggctgaggcaggataattgcttgaacctgggaggcaaaggttgcagtgagccgagatcgtgccactgccctccagcctgggctacagagcgagactttgtctcaaaaaataaataaataaataaataaataaaaataaataaatcagaggtgtaggctctgctcagttctgtgtggctccagagccttcctctctccaggacaccaagctgaaccccatctctaggccctcacccctttcccaacatctgagaattcagaaagcagatcaaacattctattgctctgtgaccttcaagccctgtagcagcctggggaacagtcataaggacttcagtacttggatgtaagacaaattttgttcatttgttcatttatttgctcttgagacagggtcttgctctgccgtccaggccagagtgcagttgcatgatcatggctcacttcagccttgacttcccaagctcaggtgatcctctcacctcaccctcctgggtagctgggacggtaggcatatgccaccacgcctggctaactttttatttttgtagagagggggtttcaccatgttgcccaggctggtctcaacctcctgagctcaaatgatctcctgccttggcctccggaagtgttgggattccaggcatgagccaccgcacccgccataagacaaagttttgtctttgagtccacgtggtgttcacttggctaagatatgtgtcttrctttctttctgtagttgatttccttcccaccactgcccagcccaccaagaagtccaccctcaagaagagagtgtgccggttacccaggccagagacccagaagggtgagttccctatccctctgcaccctcagaaacaaggcagacatccccttgccctccctccctcccctccttccttccccaggcactttccaagtgtcaactctagagcctatctccccggcctaagctagggtcagaaaggatgctgagtgtgccagcaactcacagtctcgtgggagacagactcataaattgttactggacaaagcttaatggaggagatctccacccacacccctgcacacaaatgtccatagcttcattattcatactagccaaaagcagaaactacccaaacatccaccagctaatgagctgattaacaaaatgtggcttatttattatcgtggaatattgtttggccataataaagaataaaatgaaaggagccagtcacaaaaatccatatactatatgattccatgattatggaatatccagcatatgatcctctgtagagacagaatgtagattggttgctgcctaaggctgggggcttgggaaagaaatggggagtgattcctaatgggtataaggtttctttttaggggaatttaaatgttctgaaattacattatgataatggttgcacaactccatgaatacactaaaaaccatgacattgtacactttaaacaaggggactttatacgtaaattataactcaataaagctgttaacaaacaaaaaatccactcaaaggaccagcactttaaacttttccagttttggccagatgtggtggctcacacctgtaatcccagcactttgggaggcccagatgggtggatcgccttgagctcaggagttagagaccagcctgggcaacacggtgaaactctgtctctacgaaaatgtaaaaaaattagctgggcgtggtggcacatgcctgtagtcccagctactcaggaggctgaggtggatgccttgagcctgggaggtggagattgaagtgagctgagatcctgccactgcactccagcctgggtgatagagccagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttaaggccagttgcggtagctcacacctgtaatcccagcactttgggaggctgaggcgggtggatcacttgaggccaggagattgagaccatcctggccaacgtggtgaaaccccgtctctactaaaaatacaaaaattagccaggcatgacggcggacgcctgtaatcccagctacttgagaggctgaggcaggagaatcgcttgaacccagaaggcggaggttgcagtgagccaagattgcgccactgcactccagcctgggcgacagagcgagactgtgtgtgacagagcgagactctgtgtcaaaacaaacaaacaaacgaacaaaccctatatgtatatataaaaattacataggcatggaaagagaaagttactaaattatcagtggtgattaattctgtgaaggagcaaagatgtcagggagcactctaatcctatatttaaatgtcttacaatgaaaatatattcagctatttcttgtgaaattaaaagttttaaaaataaatagtaaaagactcattaccaggcatgaaaattgtctacttcttaaaaatttgaatgtgggacatgaaattttataataaaatgatttcaataatgtttaaagtgcttacaaagactagacgatggccggacatggtggctcaggcctgtaatcccagcactttgggaggccaaggtgggcggattgcttgaggccaggagtttgagaccagcctggccaacatgataaaaccctctctctactaaagatacaaaaattagccaggcatggtggcacatgcctgtagtcccagctactcgggaggctaagaacgagaatcccttgaacccaggaggcggaagttgcagtaagctgagaatgcaccactgcactccggcctgggtgacagagcaagactctgtctcaaaaaaaaaaaaaaaaaaaaaaaaaaaatacttgaggaaaaatacattacatcgaaatacaaatggtggtattctctgggaggtggggcttccagttgaatttttcgctccttgtatttttctgcgtgtctcatgcagaaaaatgttccgtgttgaacacatgtcacatgtgtgttttccaggcccactttgtagccccatcacccttggcctgctggtggctggcrtcctggttctgctggtttccctgggagtggccatccacctgtgctgtgagttgtctcctcttgggttgttgggtgtccctgtttgctgtttgaagtgtccctgggatattttcttctctaggggagcagagcagatggcagcctggtgggaatttgcctcagagaacctacggtatttccagaggtagttcttggcctggggctacgcagccaaaaccaaagactaccctgtccctggagtcaggcaaacctgggtttgagtgcaacgtggctgtttcctgatccagtgccccggtctctctgagcctcggctgcctcagatgggggtaggacattagctacctcccagagttgctgtttgtaaattcctaccccaaatgcctggcacccggcacgtgtcagtgagtgggaactcacgtgagcagttgagaccaaagcccagtgtccagaaaagtttgcctatggggtttgtgttgtgttcctagaagctagaaatcctctccacgacaccgatattgatgaggctgtgggaaacagaaatgactgctgctcccagccaccccagagacgggactgtctccttctatggcccccaccccaagctctgcacagcactgggttcacaagaaagacggggagccagaggggaggcctgcatggctgccctagagcccagagcccagactctaccccggttcttctgctcacctgtgccacctcgagcaagccccttcccctctctgcgcatctatgaaatgggcaggtgggacagcaagggcatgaactagatcagtggctcttaacttttgtaggtcacagacccctttgaacatacgatgaaaataacggacctctccccacagaaatgcacccaggagcatacttttgcctacaattgttccccacagaggggcccatgtaccccattctaagaaacccataaatttcagtggtttgcaaactctctcgatcaaggaaggaactcattggtaaaatattttgaaacacatttaataacatgcataaaatattaaataagcgaatacagctaattgaatattataaagtaaatgcagcagttggctgtatttacttatttaatattttatgtatgttattaaatgtgcttcaaaatgaaaagtaagttgggtgtagtggcaagtgcccgtactcgagaggctgaggagggaggatcacttgagcccaggagttcaagtctagcctagacaacataataagaccccattcctaaaaaaataaatgaaaatcaaaagtaataaaggctgaaatacaaatgtcaatagacatgcttttattttcttcctaactcccagtgggaaatctcgctcaccctctgaatgcaattgccctagcctgagactgcctgtctctgggacctttcatgtctgacctgctgtgaagggcaagcaggtgtgtttaaggacacagactctgcagctgactaattgagtttgaattctggctttcccacttactagctgcatgactttgggcatgttatccagtaattcactcactctgtgtctcagtttcccacatataaaatggagatcttaaaagtagtatccaactcaggttttcagtgagcattaaggggattaaaatgtgtaaagtgtttgggccagggtctgggaccagggaggtgggctgaggtgtcagctgcccctgtgacaatcctccttggcctctggggctcagcgcagtgacccacagcccacactgactggcgcccttgctttcctcttccaggccggcggaggagagcccggcttcgtttcatgaaacagtaagtgtataacctgggtgtggccttgggttcctcagcccctgctgcagcttgcagcctctaactgcagcgaggagccaaagtcagacctcatcttccaaagatcatagactcggccgggcatagtagtgcacacctgtagtcacagctactcgggaggctgaagcaggaggatcacttgagcccaggagttcaaggctgcagtgagctatgatggtaccactgcactccagcctgggcaacagagtgagactccatctaaaaaaaaaaattacagactgtataaatagatggggtgatagagttcaggaacctaagagataacctaaacaaacaaaaacctgaccagtaagttaaatgtatttcaagaacattcttagctctcctgaggagagcaatcaggctttgcatgcacagaataccgcagagtatcccaaactcatcccatccgttatttcatcccacctattatttcatccccttggtactgtgtgagataaggggttatcaccctcatttgacagatcagtggagagactactggaatcggatttgaagaccagcctcttctaccagccattgctttccttccctaggagctgcctttgtcccacctgctgccctcgaccccatctgggtcccaggagttgacagagtcacatagcactcttggggcccactaagctgagaaagagcctgtacttaccctgggctgggctgggacctttagtggtggccatttagtcaccacctttgcaagttgctttgccctggtagggcagtgacattaggtcctgggtctttcatgaggctgttggaagccatcctgggccagggcagcattcctgcatttgatttttgttcctcacaacagagactgtttcacatctgcctatccagttaattacatttgatccttagaacaaccccctctatacagaaatgtttgggggagcaaaagtacacaactctaaattgaagagggcaacaggttctcctgctcaaaagctccagctttaggtggagacagggctgggtttgaatcccgtctgccaatcaccatgtgacagccagcaagtcacttcacgtctccaagtctcggatttctcgtctgttaaatgagctaacagtgccaatctcacagtcagtgtgaggactcactggggtattatccttaaagcatgtggccccaggaccccggagtcagagcacaagctcaagtcacttcagtaaattccactctgcaagtgcagcaatggacaaacaaaatgtatttttgaatactctaaaattgtaaatagccatataaattcatattactatatgctctgcattgttgtacacattatatttatatcgagagtggcttaaacatgcctgctggattttatgggcaattttcaatgatattttaattgtgtacaattttcaccttgtgtgttggatttagaagctgccccctacccaagagataataacttgagtgtagctgtctcaggccagacctctagaaagttccattcaaggccgggtgcagtggctcaagcctgtaatcccagcactttgggaggcccaaggcaggtagatcgcctgaggtcaggagtttgagaccagcctggccaacatggtgaaaccccatctctactaaaaatacaaaaaattaactggacgtggtggcaggcgcctatgtaatcccagctactcgagaggctgaggtaagagaatcacttgaaccccggaggcagaggttgcaatgagcagagatcacaccattccgctgcagcctgggcaacaagagtgaaactctgtctcaaaaaaaaaaagaaagttcctttcaaaatgtctcccatgatgacctttcatggaaagtaagtccctctctggtactaactgtggtctgggtctgtccccaccccttcattttaacttgctccccagtgatctgatgccagttttggggaggctggatgcaggacctcatgcttttgcaagcccaggatcagcttgaggcagggaaggggctctgggtggtcgtggggggaccctgagacctcctcactctcttcattttgaacagacgtactctcttctattgtctgggtttacacttacctgtaacatgtcatttgaacaaaagggtttgtggcttcaaaaaagcttgaaaaccacctctggttttgctctcatgatccaggcatggaggaaatagcctgtggtgctcacagatctgagttgcgatttgaggtagttgttccccagcctctaactgtccttagaaaatcatcccatctgcaggctaatttcttctggccccttgatataaatttgtgattctcttagattatcttagaggtactacaggaagtaacccaattcaaagctaccgttctttgagctgcagttttgttttgttttgttttttagccttaacaatctttttaatgactaagatgggccctagaaacatactaacaaaaaactctcggccccaggctacttggagtagaactctgcattctcatttgccatttgtctctgttacaggaaattcaatatcgtttgcctgaaaataagtggtttcacaacttgctgttgttttcagattttacaaatragcagagaatacggttttggtgtcctgctacaaaaagacatcggtcagtaacgagcacgatgtggaaaaatgagagaagggacacattcaaccctggagagttcaatggctgctgaagctgcctgcttttcactgctgcaaggcctttctgtgtgtgatgtgcatgggagcaacttgttcgtgggtcatcgggaatactagggagaaggtttcattgcccccagggcacttcacagagtgtgctggaggactgagtaagaaatgctgcccatgccaccgcttccggctcctgtgctttccctgaactgggacctttagtggtggccatttagccaccatctttgcaggttgctttgccctggtagggcagtaacattgggtcctgggtctttcatggggtgatgctgggctggctccctcttggtcttcccaggctggggctgaccttcctcgcagagaggccaggtgcaggttgggaatgaggcttgctgagaggggctgtccagttcccagaaggcatatcagtctctgagggcttcctttggggccgggaacttgcgggtttgaggataggagttcacttcatcttctcagctcccatttctactcttaagtttctcagctcccatttctactctcccatggcttaatgcttctttcattttcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctcagcactttgaaaggctgaagtaggaggatcccttgggcctaggagtttgagaccagcctgggcaacatagtaagacctcatctccacaaaaaattaaaaaattagccgggcaccgtggtgggtgcctgtagtcccaaatactcgggagtctgagatgtgaagatcacttgagcccacgaggtgaaggctacagtgagccatgatcgtgccactgcaaacaccaaccagcctgggtgacagagtgagactgtctcaaaaaaattaattaaaaattttttttaagttttaaaaaatgattgtgcaaaaaggtaaatacagtatgattctacttgcaggaggtaccaagagtggtgtctctgaattcgtcagagaaactgaaaatatcatagcggttacctggaactgggggagggggaatggggagttggtgcttagtgggtacagagttttagtttaggaagatgaaaaagttctgtggatgcatggtggtaatgattgcacaaaaatataaatatacttaatgccagtggagtgtatacttagaaatgactaaaatggtaaatttatgttatgcatacattttgccacaattttaaaaatagcttttaaaaaatggttgtagagaaagatataaacacattaaacaactgcagaatgatgagacaacatatgagtaagtggcaccaacaacatgagataagaagtgacacatactgcttgattctggctcccaaaaagtccagtgctgaaggccatccctggagtggagtgtggctttgcactgaacattgctctctgggaagggaagtgagttatttttaagctttccatttgtaaaggtgatataactgcagtaatacacattgcaaaaattagccactagaaacaacagcaagggaaacgcatcttggtgtattttcttctaatgttttaaaaacattttttacattttacatagttataatcatgctgaatgtatgtttatatcaagcatgctgtctgcgttttccttgtcaacctttccataggtttcataattctaaaacatcatcatgttaatggcctaaaattatattgaatgcattatcattgtttacttaaatattgctttatttctgaacatttccattgcttttaacatttaacatctctgttcatactctattgtgccaagactaagattacatccactggagaaatgttccagaatggactactgaatatacaggtataaacattttgatagcttctgatattgctttctgaggtcatcatgactcttacctgaaacagatatcatcttttttttttttcctgctggggtataagtggaaaacacattttcagcactgatataattttgtgttagaagattgcagcactttaaaaccctttttcccaagagctaaaggagagttgatgctataaatatggcaggaactcaaaaccctgggtacagtccctggttcatctctgccctgtgactggggcaagtcagggcaggactgttgggagctgcaatccaagcacagagttgctcggagttgcaaaaggagaagccacagggcagacatacaatacagaacctgaaagaagtgcccaagcctgcgccagcccttcaaaatcagagaagagcgggcctcgaaaaggcaaagaaagaaagaaggcagaacgagctggagcgaaggaattaaccagggggaagagaagggtggaggaagcagggggctcggaacattccctttggagaggacggggtttgtgggggctttcccctgctgcagcggggctctttgctaaaagtgaaactgcgattttcggagggttccgaaggcgttcacctagggaaggaaaatgttccatttgggggtccacactgggttaaaaacaatgcctctgcctgctctcagttatggaggcccccgagtgtcttcagtcaactctgggtggttctgaggctacagcaacaaccgaactcggcctgtgccccaacttggcccagggcctgaaggagccccagtgtgatcttaactcaagtgtgtccgcaggacaaggccccatgttgccttcccggccacaatacaacgaagaacaaagctcccgccggctgagtgcctcacaggcgccagcactgtgctgggagttgtgcgttctttttcctgttggtttcttaaccgcagaacgaggtggatattagcaactcctttagcagggctcaatggcgtcttagaaatcacaccacatttgataaatccgaaagcttcgattcaccccaggcggcctcactccaacgccttgagaattgaaggagcagcaggcttcatgagtttgatgaaaaacttgggagaatgccagtgtaacctaataaaaagtcaaagttattggattatttgaaaaaaaaaaaaaaagtaaagttgatcctttcaagagtacctatataaaaagagcacctatattcaaagccagctacaccgcctgtggttcagaagtggggagttgggtcagaaaaggtgggacagacaggatggctcagtaattagggctggcacatagcagatctgggctacctgtgtgactctgagcaagtgaattaacctctctgaaccttgaatataaaacaagaataatgataatatctacctcacacatgagcattaataaaatagtgcttgcagagtgcatggcacatactactcagtcaatggtagctgctagcattgttatcttaatgatcactgttattacattatattctagttattttttatatttttgagactagcctaaatctcaccactcaggctgcagaaaggatgtttcactaaccttggaaattccagtataggcaaacctcagagatatctcaggttcagttccagaccaccacaataaagcaaatatcacaataaagccagttacattcattttttggtttcccagtgtacataaaagttatgtttaccctataaggtagtctactaagtgtacaaagacattatgtctgaaaaaaaaagtacgtcttaatcaaaaaataatttattaccaaatacatgctaaaatcacctgagctttcagtgagtcataatttctttgctgatggagggcatcttgccttgatgtcaatggctgctgactgatcagaatggtggttgccaaaggctggcgtggctgtgccaatttcgtaaaataggacaacaacaaatcttgccacattgattgactcttcctttcacaaaagatttatctgtagcatgcgatgctgtttgatagcattttacctacagtagaacagcattcaaaattggagtcactcctctcaaatactgacgcctctttctcaactaagtttatgtaacattctctatcttctgttgtcatttcaacaatgttcacaccatcttcaccaggagtagattccatttcaagaaaccaccttctttgctcatcagagagaataaactcctcatgtgttaagtttgatcacgggattgcaacaattcagccacgtcttcagactccacttctatctctagttctcttgctatttctaccatacttgcagttacttcctccactgaagtcttgaactcctccaactcatccaggagaattggaatcacctgcttccaaactcctcctaatgttggtatttatacctgctcccatgaatcacaaatgttctttgttgcatctagaatggtgaatcctttccagaaggtttcacccagatctatcagaggaatcatcatctgtggcagctctagccttacgaaatgtatttcttaataagaattgaaagtcaaaattactccttggtccatgtaaaatcaatgttgttttaccagtcatgaaaaacaatattaatctccttgtacatctccatcagatctcttgggtgaccaggcacattttcaatgagcagtaatattttgaaaggaatcttttttcctgagcgagtctcaacagtgggctttaaatattcagtaaaccatgctctaaacagatgtgctgtcatccaggctttgctattccatttacagagtataagtagagtagacttagcataattcttaagggacttagggtttttagaatggtaaatgagcattggcttcaacttaaagtcaccagctgcattagcccctaacaagagaggcaggctgtcctttgaagccaggcattgacttctctctagctatgaaagacctagtagcatcttcttccaatataaggctgttttgtctacattgaaaatctgttgtttagtgtagccacctttgtcaataatcttagaaagctcttttggataacctgctgtgctgcagcacctattagtttctatattagcacctgcttcttcacgttgcattttgatgttaaggagatggtatcttcccttaaaccacttgaatcaaccaacctctcctagcttcaaacttttcttctgcagcttcctcaccacttcagctttcataaaatttaagagagttgggactttgctctggattaggcttttgcttaagggaattttgtggctgttccgatttacccagaccactaaaactctctccatatcagcaataaggctatttcgttttcttatcattcatgtgctcactggattagtacttttaatttccttcaagaactttttctctaccttctcaacttggctcattatttgacacaagagacctagctttcagcctgtctccactttcagcatcccttcctcactaaacttaatcatttctagcttttgatttaaagtgagagatgcgcaattcttcctttcccttgaacacttagaaaccattgtagggttattaattggcctaattttaatatttttgtggctcagggcataggggggcctaaggagaaggagatacagaatggctggtcatggagcagtcagaacacacccaacatttatggatcaagttcactgtcataaatgggcatggttcatggtgccctaaaacaattaaaatagtaacatcaaaaataactgattacagtcaccagaacagacacaataataatgaaaaagtttgaggtattgcaagaattattaaaaggtgatccagaggcacaaagtcaacacatgctattgaaaaacagcattgatagatttgctccaggcagggttgccacaaactttcaatttgctaaaaatacaacatccgtgcagcacaataaaaaaataaagtgcaataaaatgaggtatgcctgtactttaaacagtgcctggaacacagcaggtactcattacatacagttactctgttaattatgaatatatactttatttatttatttttatttatttatttattttgagatggagtctcgctctgtcacccagactgaagtgcagtggtacagtctcggctcacagcaacctccatctcccagttcaagcaattctcctgcctcagcctcctgagtagctgggaccacaggtgcccgccaccacgcccggctaatttttatatttttagtagaaacagggtttcatcatgttggccaggctggtctcgaactcctgaactcaggtgatctacccatcttggcctcccaaagtgctgggattacaggcataagccactgcacccagccagtgaatatatatttttaatatgtaattacctcctcccatgaattatgaattatgccaaatataaagaaaactactgaaggtaacatactagcttcaagtttatttagcaaattagcagatttgctaaataaatctgtcagatttagcaaatttaacttttgccaaatttgcttcagtcttttatttcaagcaataaaatgtttgtgttgagaccccttcttcacccctattccatcctccatactgcataccctatcttgaaatttgtgggttctgcctccaggctttttttatacttgtgattctcttattatgtaagtatctataaacaatgacataataatgaataatatatggcatgtgcttttattttttataaataatgtcagagtgtacatgcacttttaaaatttgcttatttcattcaacatatttttgagatttacctgtgttgatgcctgtggatctagttcatttattgtgtcatattttattaatgtattacaattttattcattcccctattagatattagtttataattattattacaattttccaatttctgttgtttgtgcttctgtgaacactcttatacatcaaaacttctctgagcaatgaatctggaagtggaagttctggggccagagcatcttcatttttaatagatatttttactagatatcccaaattatctccactgcagttgtaacaatttacatccccatcaacactgcatcacagttatttggtcatatcctccctcatgcttgatgttattggtcttgttcattttgctaatttgaatataaaatgatattggttgttttcatttgcatctcactgattcaagtaatgtttggcatctttttatttggatattggcttggtcacgtactttgcccatttttaattctgctgtctttaacttattactttgtgagagtctgatatgttatggataaagatcttctgttgcttttatgcactgcaaatatcttctactaatttgagacttgtcctttaactttgtattggtttttgccctgcatactttaaacaaatgttcgaatggtaacatcacacttttatggcttgtccttttatattttgaccaagaaattctgtgccttatcatgtctctgtcttaaaaacacattttttaaaaaagttttgaaactttgcttttcatatttattctatctggagtatacatttgtgaatggtttgagatagagttcaagttttacgtttttcccacatgggaagccaatgtcacagcactaatgatcgaatcctttacccacaggagtgcccagccatctccaacatgttcacatctgtattttctaggccccctactctctttcattgttctatttgtgtttaaattactagagtttataaatcatgatatgtggaataatgggtcccccccttacccttcagcaaaattattcagctatttttggccctttatacttccatatgaatttgataatcagcttgtgagcaaaatctggttggcattttgaatgcactggatctgtagattaactggggaaaacttacatcttcctgaagttgagtcttcatgtccattataaagtaccccggaggagaagtttccatcagaataaattctctaacatttgtgaccatcttgtcttccaacctgtgatgacaatagccaccctaccttactactctcctgagagaaattttttaagcatggggacctctctgaagtctcctgcagctgttgacccgtacatccttgattccaaatggcagaaaactaatttgcaggtcagacactattgaaattcaccttgattaaccatggcaataaaattgacagtgatgcccaagagggaaggataaaaccctcacccgctttcccaaatgatattaactttggggaataactgagcaggtaattctctgtgtgtggtttcctctttctgagaagaaatcacaagaaacagaaacagaggtttctggctttgagatcctgaatcaaatgtggttggatcctgtggtaaccctaggcctatcaagtgctatatgcctggctctttctgtgttgaatttgacacaacatctgactcgactgagtcacagaaaaaaatcagtcattacttaataagtaaccaacaccttgacttggtgcatgtattgagactctcttgtttttctttttgcaaggcctcaaggggaaggtrtatcaggaacctttgtcccccaatgcctgcatggatactacagcaatactacaacctcacagaagctgcttaayccatggatcctgaaaacataggcaagaagcacaggtcctgatgagtggatctttactacttttaccagattcctcyctcgctcagaggatcctcccacacacccagcagagacttctctgccaccatcaaccccccaagtcatagggggctcaagttctgccctggtgagctgtggcccccatccttgtgaaccccaaagtgtcccccttgtggaaccaaatgtatccatcttgaataaatttgcccaaaatcctaaagaccgggaaaagggaaccattgagcttggcaagtactggggaacatctttgcttcctgcatctctagcaccagatcactatgagtatggaccacccatttcagaacagccgccaaatgctgaggttagagagggttacttatgtcaggcagatgttctttccttgtgaaggaaagcgtttttaagcttctttgctttttaaaaatttattttttttatttatttatatatttttgagacggagtctcgctgtcacccaggctggagtgcagggactcgatcttggctcactacaatctctgcctcctgggttcaagtgattctcctgcctctgcctgctgggactacaggcacatgccaccacacatggttaatttttgtatttttagtagagacggggtttcaccatgtttggccaggttggtctcgaacccttgacctcaggtgattcaaccacctcggcctcccaaagtgctggaattacaggcatgaaccacccaggcccggcctacttttaatttattttttatttttgagacaaggtctcactctgtcacccaggctggagtgcagtggtgcaatcacggctcactctagcgttgacctcccaggctcaggcaattcttccacctcagcctcctgggtagctgagactacaggtgcacaccaccacacctggcaaatttttgtatttattgtagacacggggtctccttatgttgcccaggctggccttgagctcctgggctcaagcaacctgcctgcctcagcctcccaaaatgttgggattataggcatgagcaccatgcccagccctttttttttttttttttttttgaaatggagtctcactgtgttgcccaggctggagtgcagtggcgtgatctcagctcattgcaacctccgcctcccaggttcaagtgattctcctgcctcagcctccagagtagctgggattacaggtgcccaccaccacgcttggctaatttttatatttttagtagaggcagggtttcaccatgt

SEQ ID NO: 2 - DNA, 14483 bp, Homo sapiens
  allele (2195)..(2195): PS5 polymorphic base guanine or adenine
  allele (2197)..(2197): PS6 polymorphic base cytosine or thymine
  allele (2382)..(2382): PS10 polymorphic base guanine or cytosine
  allele (5804)..(5804): PS13 polymorphic base guanine or adenine
  allele (5809)..(5809): PS14 polymorphic base thymine or adenine
  allele (6196)..(6196): PS16 polymorphic base cytosine or adenine
  allele (6688)..(6688): PS17 polymorphic base adenine or guanine
  allele (8299)..(8299): PS18 polymorphic base cytosine or thymine
  allele (8462)..(8462): PS19 polymorphic base cytosine or thymine
  allele (8498)..(8498): PS22 polymorphic base cytosine or thymine
  allele (11587)..(11587): PS32 polymorphic base thymine or guanine
  allele (11963)..(11963): PS38 polymorphic base guanine or thymine
  allele (12174)..(12174): PS40 polymorphic base guanine or adenine
  allele (13575)..(13575): PS43 polymorphic base cytosine or thymine
  allele (13914)..(13914): PS47 polymorphic base adenine or thymine
  allele (14309)..(14309): PS50 polymorphic base cytosine or guanine
cgccctttcaactctgccaagaatggctcccacctggctctcagacattcccctggtccaacccccaggccatcaagatgtctcagagaggcggctagacacccagagacctcaagtgaccatgtgggaacgggatgtttccagtgacaggcaggagccagggcggagaggcaggtagggatccatccacgccgttttctcaggcttgcttgctagtgacccttcctcactggaataaactccttaccctatttcggccctagtttccagaacgtactcatttttatgtaagcaattagttcctacagtgcataaataataaagcagtgtgctaagtgctagggatgaggataacaatagtgaataagacaaatcctgacttaaatttgtaattaataaggaatctaaaatcctaagaggggagatagattgaccaccagctgatggtaaagcatggtatgaggcaagatgaagaagtgcaaactagaggtagagacccagcactgtggcagtgtgggttcattctgatggaagaaagcctttacagaggacatgctgtgggagctggccatgctgtcgtattatctgtaaacattcttattcttgtttcatgtgtcttttcccagatgtactgggatatcattctgccattttgctccagaataggattacattagaagaagcagcatgatgaagaaggaatactagactgggtgtcaggagacagagactctatttctagtttggctaccagctagaggacatttggcaagtcacttaatttccctagcctacagatcgctgctactaaagaaaagaaagaaagagcgagccagatatggcggctcacacctataatcctagcactttgggagctgaggcaggaggatcacttgagcccaggagttcatgaccagcgtgggcaacaaagcgagactccgtctccacagaaaataattagctggctgtggtggcatgcatttgtagtcctagctactcaggaggctaaggtggtaggaccccttgagctcagaagttgaagactgcagtgacctatgatccagccactgcattgaggcctgggtgacacagtgataccctgtctctaaaaacgacaacaacaacaatctcttatagtcctgggtctcagagagctgcctcaggagccatgttccaagctggattaaacttcacgtgacattggtagacgatttctctaaaggctggcactgtgttatttatgtactgttctctcagactcctcataaagtatacctaacactcaataaatgccttttttttttttttttttttttgagatggagtctcactctgtcgcccaggctggagtgcactggggagatcttggctcactgcaagctccgcctcccaggttgacgccattctcctgcctcagcctcccaagcagctgggactacaggcacctgccaccacgcccagctagttttttatatttttagtagagatggggtttcaccgtgttaaccaggatgatctcgatctcctgacctcgtgatccgcccgcctcggcctcccaaagtgctgggattacaggcatgagccaccgcgcccagccaataaatgccttttaactagcacctggcctcaccatattgatactggaagcttacgacctctctatgcccattcctccctcaaacttcttgcccttaaattagaattgagaagtccctgtgtgttcttccaacctttccacttaaaatgtgtggcctaaaaataaaaaaataaataagacaaaaaaaccaccaaaaaacaaaaagaatgtgtggcctaatgatatacacatttgatgttgaatcctcctgtatgtagctctctctagggagatcatgttccacgatctcagccagactttaggtcctgtgagtccaggcactggactcaacatgctcaatagggctttgatgaatgatgatgatgtcaatgcagacatccccataccccagcttcagcacccccttcacctccccacacggaagcagaggggtcctcttttccttctcctggctatgtttatgccctcaactatccttccagcactggagacaagtctcacctgcactaacctgtctttgaaggtcctgggggctggaggggtcacaggccctgagccagcaggctgaggtgatcgttcggcagctgcaagagctgcrgyggctggaggaggaggtccggctcctgcgggagacctcgctgcagcagaagatgaggctagaggcccaggccatggagctagaggctctggcacgggcggagaaggccggccgagctgaggctgagggcctgcgtgctgctttggctggggctgaggttgtccggaagaacttggaagaggggagscagcgggagctggaagaggttcagaggctgcaccaagagcaggtgaatgcaggggtagaaaggattcaaattcataacggagagctgggcagtagcttccaagcaaagaacaggtattgcagaaaagaccctccatgagtagtgagtagtagagtgatgagacctttggtgaaaataaacacacatgggctagaaagatggagaatttgggtacttttatcttaattcaggttgcacttttcccccaagactcaggtggcctcccaccacttgaagccctgcttccctttctagtaggaaatagtttgcctccctactttactccaggtaccaattcatcaggggtattgtggagggcagtgaggaaggtgggtataagggggctatggtggactgggagagagagattattcagtcctcaaactcagtacttactctgccatctctaagactcagtgaccaaattagcctgagtcctgccttcgtggatggaactgacagcctcgtgggggatatacacatccacatttaatttaatcataattaattgcaatcaggaggaatgccttgaaggagaaggactagaaagcactaacttagtctggggttcagggaaggccactgtaaccagttgacatgtcaaggaaagaaaccatagccctgggcgcagtggctcacgcctgtaatcccagcattttgggaggccgaagcaggcagatcacaaggtcaggagttcaagaccagcctggccaacatggcgaaaccctgtctctactaaaaataaaaaaattcaccaggcatggtggtgcgcacctgtaatcccagctacttgggaggccgaggcaggagaatcacttgaacctgggcagtggaggttgcagtgagccgagactgtgccattgcactccagcctgggcgacagagcaagactctgtctcaaaaaataaaataaaaaaggaactgggagaaaacaagggagaattcctttataaccttgtagtgggcaaggcctttctacctgtgagtcaaaatccaaaatctagaagccataaaggaaaaaattgatccattgactttataacatgaacattaggaatagccaaaaagaaaaaaaaaagctatatttatagctcagatcacaagaaaagggtaatatccctaatataaaatgtgtgcctagaaattggtaagggaaagaccagcaatccaatcagaaaatggacaaaggagatttatgaaagaaacttagaaacaagaagctaggccaggcacagtggctcatgcctgtaatcccagcactttgggaggccgaggtgggcggatcacttgaggccaggagttaaagaccaacctggccaacatgaagaactctacaaaaaaatacaaaaattagccgggtatagtcgtgggcgcctgtaatcccccagcttcttgggaggctgaggcaggagaattgcttgaacctgggagacagaggttacagtgagctgacatcacactccagcctgggcagcagagcgagactaaaaaaacaacaagctaccgtttgtgctgaataggagttggccagtgaagaggcgtgtgaagtccagtggtagctggaagacacttggtgggacaacaggtgaaggcggggacaggaggccagaaggctggggcacagagatgaggggcactgagtgtgctgcagagcccaggacccagggcacaaggctttggccacttcagaacttgctactttcccataagagcaatgagcaggctgggcacagtggctcatacctgtaatcctagcactttgggaggccaaggtggaaggatcatttgagcccaggagtttgagaccagcctgggcaacaaagcgagacccccatctctattttatggaagaaattagggctgggcatggttgctcacatgtgtaatcctagcactttgggaagctgaggcgggtggatcacttgaggtcaggagttcgagaccagcctggccaacatggtaaaacctcatctctactaaaaatacaaaaattagctgggcgtggtggctcatgcctgtaatctcagctactcgggagggtgaggcgggagggtgaggcaggagaatcgcttgaacctgggaggcagcgtttgcagtgagctgagatcgtgccattgcactccagtctaggcaacaaagtgaaactccatctccaaaaaaacaaacaaaaaaaaattgttttttcaagtaataagcaaccgttgaaaggttgtttttttttttttttgagatggagtctcgctctgtcgcccaggctggagtgcagtggcgcgatcttggctcactgcaagctccgcctcccgggttcatgtcattcttccgagtagctgggactacaggcgcccgccaccacgcccagctaattttttgtattttaatagagacggggtttcaccgtgttagccaggatgatctcaatctcctgaccttgtgatccacccacctcggcctcccaaagtgctggaattacaggaatgagccactgcgcccggcctgttgaaaggttttaagcagggaaataacatgattagatttgtattttatgtctaaaaaattttgtcatttatgtcccccaaattaattttattgttgtatggagacagggctagaggaggcagaccaggaagcagggtgggcactttgccctcctttccagtccatcccatgactcttggtggctctgacacccctgcaacactttgaggtgccatgagcaaaagacacaaaattcctcctttcctggagctttccttccagtgtggtccgacagatagtaacacatacacataagcaagatatggtcagtgctaagtgctcaggaggacgtgaacagctgatggggcagagtagggtggggagggacagtattagagggcccagtgaagccaccctgaggaggggctatcgcctggggtctgtggagcaaggaggggccgctgtctggttctcagcagactccccgtggccggagcggggagcagtgggagagcctccagggtgagctcaggaggtaggcagaggccgggtcccctggcctgcaggtgtggagagacacctgggttttgttgtgaatgctgtgagaagccactgagggtttgtaaagactagttaggagatggtcgctgttgcccaggcaaaagatgagggttggtggcagtggagacggagacagagaggtgaagatatgttttgggggagatcggacaagaactcctgatgggttgtggggcagctgcggagagtgagttgccagctctccatttgctgtgcacagttggctgattggttgggtcattctctaaggtcacagaaagtgggagtgaagggaacaaggaaggcctccatgtggggtcgagcctctgctgagccccctcttctttccrcagcwgtcctctttgacacaggctcacgaggaggctctttccagtttgaccagcaaggctgagggcttggagaagtctctgagtagtctggaaaccagaagagcaggggaagccaaggagctggccgaggctcagagggaggccgagctgcttcggaagcagctgaggtaggtgggcggacgccgacgggagcccagcaattagtgatgtggtggatctgcagggcgccccactgatggctgtcccattcccaccccaaccctagcaagacccaggaagacttggaggctcaggtgaccctggttgagaatctaagaaaatatgttggggaacaagtcccttctgaggtccacagccagacatgggaactggagcgacagaagcttctggamaccatgcaggtgagggtgcaggaatgtatctgtgtgcagacttagggatcaggttgggaggcaagcgtggcccttggaggagcgtgtagagcacagcctccgggagagaaggtggtacctaaggcggcatggaggccctacagaggggctgctttcctctgcccgcagcacttgcaggaggaccgggacagcctgcatgccaccgcggagctgctgcaggtgcgggtgcagagcctcacacacatcctcgccctgcaggaggaggagctgaccaggaaggtacagcccaacccccagacccctcaccctcagccgcatcctgcatctactgtcccctgcctccctccctgtgggcaggaggggtcaatgtgccccagaacctgcttagatctccttcctgtgaactcctcttgctgtagctcatgttgcccaggcaggacagaggagaaacaaagatgccacctccttcctctcctcccccaggagcccacrcttttctcccactccttctccctcaggttcaaccttcagattccctggagcctgagtttaccaggaagtgccagtccctgctgaaccgctggcgggagaaggtgtttgccctcatggtgcagctaaaggcccaggagctggaacacagtgactctgttaagcagctgaagggacaggtcactgcactctcttttctcccggtattccctcccagcaccttgctccttccatgaaggtggcatccattcaaccagtgtttattgagtggttgccacatgctgggcacacagccctgaacaaaactaaaatgtggagcttgcattctagaacagagacacagaacacgcaagtaaacagataatgttgggtaattatatgtgcgatagaaagattgaagccgggtgcagtggctcacacctataatgcgatcactttggtctccaactcctgacctcaggtgattcacctgcctcagcctcccaaagtgatgggattacaggtgtgagccaccgtgcccagtcaagtaatgccaacagtttgggagaccgaggcaggtggatcactggaggtcaggagttcgagaccagcctgggcaacatgtgaaatcccgtctctactaaaaatacaaaaaattagccgggcatagtggctcattcctgtagtcccagctactctggaggatgaggtgggaggatcacctgaggctgggaggtcgaggcgaggccacagtgaactgtgatcccatcactgcactctagcctgggtgacaaagcgagatcttttctcaaaaaaaaagaaagtagtaagaaaaattcaaaagataatgtgacggagagactgtggggtgagtcagcctcaggtaggatgctcagagacagcctctctgaggaggtgacagcatctgaggagagtggcatggtcagttggtgggtcttgtggggtgtgtcaagggctattcccatcttcgagtgggcacatggaatgtggaacatggaacactgggctcagattccatcctcagaacctaagcttctgtctccctgcgtggcattcattctttttcttttcttttctttttttttttttttttgagaaggagtcttgttcttgtcacccaggctggagtgcagtggcctgatctcagctcactgcaacctccgcctcccaggttcaagtgattctcctgcctcagcctcccgagtagctgggattacaggcacatgccatcacgctcagctaatttttgtatttttagtagagacaggctttcaccatgttggccaggctgatcttgaacccctgacctcaagtgatccatctgcctcggcctcccaaagtgctgggattacaggtgtgagccaccgtgctgcgacccacccccgtcgcccgccctccctccccccagcccctgcatggcattcttacagagatctctgcacctgccactttgcttccagtgcccccctcatcttttagctctagagggccctgcccagctctctctcctcccccaggtggcctcactccaggaaaaagtgacatcccagagccaggagcaggccatcctgcagcgatccctgcaggacaaagccgcagaggtggaggtggagygtatgggtgccaaggttggtgtcagcctactagagactcggggagggcaagggagcccctgttccggggctgcagccaggacttagggagggaccctgtcctttgctgcatcctccccagggcctgcagttggagctgagccgtgctcaggaggccaggcgtyggtggcagcagcagacagcctcagccgaggagcagytgaggcttgtggtcaatgctgtcagcaggtatcagggatggaggggtgggtggagtagtgtttctgccacctcaggttcctgggcaccttgttgctgaggatcctcaggcaagaggggctggaaagtggccactggaggctacagggctgggcagatttagctctatcaatgttcctgtgttcgtttcttttcctggggaagccccttctgcattcatacctgattgcttgttatgaatttcccgttgcatgtttggctggaggtgaggccttgcttcctcctgcagttcagtctagtaatggcttgagctaaatagagcacccgggaggatcttcacttgcagtattgttcaaggatggagagtgtagacacttcatcttcctttttttttctaaaattttacgggcaatccgtttcactggagaaaaatttagtctatttatttatttattttgagacaaagtctcgctctgtcacccaggctggagtgcaatggcacaatcttggctcactgcaacctcacctccctggttcaagtgattctcctgcctcagcctcccgagtagctggattacaggcatcctccaccagtgtcctccactacgcccggctaatttttgcatttttagtagggacggggtttcaccatgttggccaggctggtcttgaactcctgacctcaggtgatccacccacctcagcctcccaaagtgctggaattataggtgtgagccactgcacctggcctagtctatttatttaaagctgtatacttacttgcttattatatacttaacttgcttactattccatctaaaatgtaagccagttagtttccttctaaatcaattgccagcctttgtctctcctaccaacttcctagttgtttcattacctacaattgttgtatgaccttcagaaaaacctctaagaaaacagcaaagcttctttgtgctggtgatgacttcccctcagccttagacactgaggtacccaaggcaagtagttctttttttttttttttgagacagagtctcgcactgtcacccaggctggagtgcaatggcacgatctcagctcactgcaacctctgcctcccgggttcacacgattttcctgcctcagcctcctgagtagctgggattacaggtgcacaccaccacacccggctacttttttgtatttttagtagagacagggtttcactgtgttggccaggctggtctcaaacttctgacctcgtgatccgcccgcctcggcctcccaaagtgctgggattacaggcttgagccaccgtgcccggccggcaggtagttcttagcacagtctctggcttgtaaatgtttattgttatcgtgaggctcttcttgatgggttaatttagataaagataatttttggtttagcgaaattaagatgcaggatgagtccttgcccaccacttccttctcttgggtttgtaccttagggactaatttagctttaaaaattatgaaaaatttcaaacttgcacggaattaaactagttataatgacctaccacccaggttcaatccatcgcgggtgccccctacctccaggagaacagaaagatgcactgtggacagggtttgactttggtaccaggtgatcacagaaatggtctgtgtaatgatgcatttgccgaaagtcctccaggcttaaagcagtccaaactctgattgttgctgggtttattagattgtctctaggtaattggagactttaataagagctgtggtaggtgttggaagcatctactggaacaatttccagatcaaagtgaactttgctgtgctgctggggatgcagctgcaagccttcatcatcatgtgtttttctgtgggtgcagacctggactctcctgaggaaaccccagcccggccccagacactccttggcctcctcctggggcctgtttaagctgctcagttttcatgagccaggttggtcctacttctggcacagccagctggtaaagcatgtggacctgcccgtcattggtgctagatcgacactcctgggcttggagaggataactttgttttcttttgtttttttgagatggagtctcgctctgtcacccagtcttgagggcaggggtgcgatcttggctcactggaacctccacctcctaggttcaagtgattctcgtgcctcagcctctggagtagctgggattacaggcatgagccaccatgcccggctaatttttgtgtttttaagtagagagagtttcaccatgttggccaggctagtctccaattcctgacttcaggtgatccgcccgcctcggcctcccaaagtgctgggattacaggcaggagccactgcccctgaccagagaggataactttactctttgatacacgatagtgagcaaaacacagttgtgagaaataagcttaacaggttgcttaaaaagatagtcatttaatgcattcttggggcaagggtcctttagataattgacggaagctgtgcgttctgtacttgtataatgggacaggattagagggagttgtctatacaaggcacagcaagtcctttgggaatgaggggaggcatggaggatcagtgacttgtgccctctccagctctcagatctggctcgagaccaccatggctaaggtggaaggggctgccgcccagcttcccagcctcaacaaccgactcagctatgctgtccgcaaggtccacaccattcggggtgcgtaggacaactgcgagccacgtcctgcccccaccccaccagctcggactttcttcttcctgacccagctctctctgatcccacatccattcaccttcctcctttcaccagtccttgcatctctttttcccttactccctgtccccactttctcccatgcaaacttcatctctttttctccctgctttttcccctccaggcctgattgctcgaaagcttgcccttgctcagctgcgccaggagaggtgaagtttgtgcactttgaggtggatggggctttagggcattggctgctgggacccccaaaaccatgaggactgaggtgggatgggggctttgggatcaggcagctgggtkatttctcctgactctttctcttccccgtcccagctgtcccctaccaccaccggtcacagatgtgagccttgagttgcagcagctgcgggaagaacggaaccgcctggatgcagaactgcagctgagtgcccgcctcatccagcaggaggtgggccgggctcgggagcaaggtacacctggttgccagagggtggagaggatgaggaaaaacccagtgtctagggtgctgggagaggcctgacccagcaccccctccttttaggggaggcagagcggcagcagctgagcaaggtggcccagcagctggagcaggagctgcagcagacccaggagtccctggctagcttggggctgcagctggaggtagcacgccagkgccagcaggagagcacagaggaggctgccagtctgcggcaggagctgacccagcagcaggaactctacgggcaaggtgtcgagagggaaatgggtgcttcccttggagggtggggtgggaactgcgaatcaaagctcctgctgatatgccccgtctgcactttcaccccagccctgcaagaaaaggtggctgaagtggaaactcggctgcrggagcaactctcagacacagagaggaggctgaacgaggctcggagggagcatgccaaggccggtgagccttgccagggtggatagggccttccaggaagaaggaagtgttaagacataaggttattattttcccctcaaagtgtgttcaaagcttcattacaggaagtaatgaaggtatccaggagtagcacagatgaattatcacatcgtgaacacacccatgtagccagcaccagattaagaaacagcatatggccggtcgcggtggcttatgcctgtaatccaagcactttgggaggccgaggtgggtgtatcacctgaggtcaggagtttgaggccagcctggacaacatggcgaaaccctgtctcgactaaaaatacaaaaattagctaggcctggtggtgggcacctgtaccccaagcttacttgtgaggctgatgtgggaagactacatgaacccgggaggtcgaggctgcagtgagccaagattgtgccactgcactcaagcctgggtgatagagaaagaccatgtgtcaaaaaaagaattgtgtaatgaatgtatcttctctaactaaatatagcagttaacatttgccacatttggtctcttatctatatacacacatatttgtacatcttttgaatcactttaagttgtaatcatttaatgttttgttgttgttgttgtttgagacagagtcttcctctgtcaccagctggagtgcagtggcatgatcttggctcactgtgacctctgcctcccgggttcaagccattctcctggctcagcctcccaagtagctgggattacaggcgcccaccaccatgcccagctagtttttgtatttgcagtagagacgggattacaccatgttggccaggatggtctcgagctcctgacctcgtgatccgcccgctttggcatcccaaagtgctgggattataggcgtgagccaccacgcctaagtaagttgtaaacataagttgttcagccgcatctcccaaagccagtaaattctcctatatagctgcaatcatcacactttaagacagtgaacactaattgcacaaaatctaacccagttcatgttcagatttcccctgaggaactccaggatggttcagggatgaggaagatacttaggttcagattcccaggctcctagagcatcagcccacccctccaactgtacagaagagacagatccacagagcagaacagcctccccaagccacagagttggtgacccagcgtttgttcctgtcttcatggtgcctggctgcctctggcctgactcacacctgcctcctctgtgccttggcctctctgtagtggtctccttgcgccagattcagcgcagagccgcccaggaaaaggagcggagccaggaactcaggygtctgcaggaggaggcccggaaggaggaggggcagcgactggcccggcgcttgcaggagctagagagggataagaacctcatgctggtaggagacaggagggcagacaggcagacactagggcccatcctgggctggttcctgggctagaggtgtggaaagaggatggtgagggaggctctatccgggctaggtttaaccctctccttcccaggccaccttgcagcaggaaggtctcctctcccgttacaagcagcagcgactgttgacagttcttccttccctactggataagaagaaatctgtggtgtccagccccaggcctccagagtgttcagcwtctgcacctgtagcagcagcagtgcccaccagggagtccataaaaggtcttgggccaagcacaaagggacaagggacaaatgcgcgcacttcaggaatctcctcttcagactctggcatgatgagtgttgttctctgcggtccttcgaggcccttagcctcttttagcgatgcccagcttggaccaaagagcctcctctctcccattcctcatttcctgtgccagccctgtttcctctgtaaccacgagcaccttcccttgtctggtgctcatctgctgtcttccttcccagggtccctctctgtcctgctcgatgacctgcaggacctgagtgaagccatttccaaagaggaagctgtttgtcaaggagacaaccttgacagatgctccagctscaatccccagatgagcagctaagcagctgacagttggagggaaagccagcctgggggctgggaggatcctggagaagtgggtggggacagaccagcccttccccatcctggggttgccctgggggataccagctgagtctgaattctgctctaaataaagacgactacagaagg

SEQ ID NO: 3 - DNA, 4416 bp, Homo sapiens
  allele (1128)..(1128): PS2 polymorphic base guanine or adenine
  allele (1402)..(1402): PS6 polymorphic base guanine or adenine
  allele (1792)..(1792): PS7 polymorphic base guanine or adenine
  allele (1863)..(1863): PS12 polymorphic base thymine or cytosine
  allele (2019)..(2019): PS16 polymorphic base adenine or guanine
  allele (2407)..(2407): PS20 polymorphic base cytosine or thymine
  allele (2873)..(2873): PS21 polymorphic base guanine or adenine
  allele (2985)..(2985): PS22 polymorphic base guanine or adenine
  allele (3159)..(3159): PS24 polymorphic base cytosine or thymine
  allele (3417)..(3417): PS27 polymorphic base adenine or guanine
gtttgagaccagccaacatagggagactccatctatataaaaaataaaaataaaaaacattggagggcatggtggcgtgcacccatggtcctagttactccagaggctgagttgggaggattgtttgagcccaagaggttgaggctgcaatgagctgtgatcgcaccactgcaccccagcctaagagagcgagaacctgtcctcactccccaaacaaagggaagacgcaacatgatttggtgtacattttttttcctgagggacttactggatggtcccttccagagtgaggtacacatatccacgcactgtggtcagcgattgctcccggcacccagcgcagcagatgggtgggtctaaccaggtcacttccccaggagggcataattgagcagtttccgcatcaggtccacgtgggccagcagcatgcggcaggcagcggctgtggtgcttcaggcccagggcgtccccatcagcgtacttggactgcagcagccccaggtaggcctcccacgtgttgcccacagtcttgctgcaggtgaagcagcgcacacaactcctgagctcaagtggtcctcctgtgtcagcttcccaaagtgctggcatgagccactacgcctggcctgatatatatatatatgttttttttttttttgagacggaatttcactcgttgcccaggctggagtgcaatggtgtgatctcagctcaccacaacctctgcctcctgggttcaagcgattctcctgcctcagcctcttgagtagctgggattacaggcatgtgccaccatgtccggctaatatttttgtatttttagtagagacggggtttctccatgttggtcaggctggtcttgaactcccgacctcaggtgatctgcccgcttcggcctcccaaagtgctgggattacaggcgcgagccaccacaccaggcccgcgtgatgtatattttaagacctcttttgctggtggaggacaggctttgtgtgagggggagggataaacagtgggagcaagggggccaattagaagggtgttggggaggctcaggggagatggtggctcaggatgatgggctgggtttggacagggtgtggaggggcttgcaggtggatggtggaggartgtaacgaaggtttctgcgtgagccctggagggaacagatgagatcacgccattgcataataaggtgttccttactgtggggtagcggaccaggcagggaacaacctgggaggaatcaaattttattttggacatgttacttctgaaaggctaacagacttccaggcagaaaggtccttgagggaaacattctaggggtctctctgggaggcttagatcaaggagctgagaccaaaaggagaatgggagggaggagacgagtacaatagagttrgagccaaggtcctagaggcggataggtggattcctgagggaggaggaaggggctgaggttgctggagcctggcagcttcttccggagccattggcaggactgatgcaaacagctctgggtgggaagagggaaccaggatatcctcctgtgtccttccttttctgcagtcatcctgggtggctgccagatggaattccttggatatcattgcttggaggtcccctgcatgcctgaagaaggacatggtggagagcaggatgcctggatcccatgggggaagggaagtgcccaggaaagcacgaagccccagggggagctttcagtgcgggggtgagtggggaggctggggtagtagctgacactgtcccagctgcatcccaggtttgaaargcacctcctcccccagcgcaggcatcctgcctcccaaccctgtaattacggtgcttcccaacgcccatcgygtggtttgctcccattctttggcttccaatagttgcaagggatgaaggtggacatctctgtgattacggagatgccaagtgggtattgactgctccaggatgtggatggagggtgtgaaaaccagggtggggtgacgcaggctctgggtcatgatrgggagagcaggcagctgggtcctgggctggaggactaaaataagggacgccaccttcaggggtgacacatcagcccaggccctccctacgggtttgaccagtcctgttctgacggtattcctgcgccactgggctggtccctcctccactcctcccctataaagcctcctggggtccccaggcacccagactcagcccaccccagctttgggggccagtacgcagccatgatcctcaactggaagctcctggggatcctggtcctttgcctgcacaccagaggtgaggtgggaacagaggcagggactgcagtttggggtgatgagggatactcaagatggcggaggtgaactggacgcatggggttggggacaggaattcaggggaygcagaaggtgcatctggctcaccagaaatggctttctggacacattgggtgggggacatggtgcagaaggtgcatttggctctcaccagaaatggtttgctggctccatgtggcaaagtcggtcaggattaacgtgggggggacgagtttccttggagctgggatctgtgttaaggagctggggtccttgtaaagctggggtctgtgtgcctgggggccaaggtgtaacccaccttgggttgcaggttggcctgaggacaaagctagtggggtaccccaaccaggggtggatggagcttatttggagaagtctggtcagtttaaagtgggtcaagtgaacggttcagatccatcgggggtaggggttcatgacattttaccatcagttaagtatttacaaacctaccgagagctctttgagagtgacttttttggtctgtttgtgggtcagttcaggctgcgtccrtccagacaggctcctcctcctggggctggggctgggtggggctggggagagaagccctcaccacctcttacctttctccttcctcctttacaggcatctcaggcagcgaggrccacccctctcacccacccgcagaggaccgagaggaggcaggctccccaacattgcctcagggccccccagtccccggtgacccttggccaggggcaccccctctctttgaagatcctccgcctacccgccccagtcgtccctggagagacctgcctgaaactggagtctggcyccctgaaccgcctagaacggatcctcctcaacctccccggcctgacgacccttggccggcaggaccccagcccccagaaaacccctggcctcctgcccctgaggtggacaaccgacctcaggaggagccagacctagacccaccccgggaagagtacagataatggagtcccctcagccgttctgttcccaggcatctccaggcacccacgccctctccaccctctgattccccgtgaattcttcccaatttagcctrtctccttaaacctcttcctcattccctcggttttattctgaacccgtaaggtggtgttctcaatatttcctgtcccctcctgagatccatacttagtcctcacatcgcccgttttttcctctgacagcctaagcctactctcctacctcgcctccaggcctcggccccacctacctcccacccggtcttcctgcccgcgcgatcgctggggcagggctacggtactgtgttcccttctgccacctggtggccggcggcaggaactatcagtagacagctgctgcttccatgaaacggaaaaataaaaatcatgttttcttaactctgaatctaggctgctgctttaactaacacttagggtctttttcatttatttttatttatttgtttttttctttttttgagacgaagtctcgctctgtcgcccaggctggagtacagtggcacgatctcggctcactgcaagctccgcctcccgggttcacgctattctgcctcagcctcccgagtagctgggactacaggcgcccgccaccacgccaggctaattttttgtattttttagtagaggcggggtttcaccgtgttagccagagtggtctcgatctcctgacctcgtgatctgcccgcctgggcctcccaaagtgctgggattacacgcgtgagccacagcgcccggcttctttcttctttttctttcttttttttttagatggagtctcactctatgcccaggctggagtgcaatggcacgatctcggctcactgcaacctccggctcccgggttcaagccattctcctgcctcagccttctgagtagctgggattacaggtgcgcaccaccatgcccggctaatttttgtattttagtagagatggggtttcaccatgttggccaggctggtctcgaactcctgacatcgtgatctccccgcctcggcctcccaaagggctgggattacaggcgtgagccaccgtgcccggccaacacttatgtt

SEQ ID NO: 4 - DNA, 6209 bp, Homo sapiens
  allele (1568)..(1568): PS3 polymorphic base thymine or cytosine
  allele (1661)..(1661): PS5 polymorphic base cytosine or thymine
  allele (2186)..(2186): PS6 polymorphic base thymine or cytosine
  allele (3929)..(3929): PS7 polymorphic base cytosine or thymine
  allele (4235)..(4235): PS9 polymorphic base guanine or adenine
  allele (4326)..(4326): PS11 polymorphic base cytosine or thymine
  allele (5210)..(5210): PS13 polymorphic base adenine or guanine
tgcagggctagaccctccccaagaccttgggaatccaggccgcctagatccccaggcagaaaagccagcgtcctgacatcttattcaaatctttcctgcggctgttctctcagcttctctctactatccccttagcttccatgcctgctgcccgcctcctctttctcgagtcctaacacatagtgggcacttgaagatcttcctcccaccctcccaccctcattaatctattttttacccgaatctgggatccctactcccgtccctttttacaacctaatctacctttttctgaaggaattattctggtcctgaccctcacccccatgtctcaatagcctgccctcgccccctgtacgctagccggctctactctcccaccactgctcccctagataccagaggcttcacccagttagcccgtgagctctaaggctgttctgatctcttcatctgtcccttcacctggcccctgtacccccttcccctttggaccccttgaaccctcccaggacccccgctcagccccttcccgcccccaaccgactcttcccgaacgtcccttaccaaccgcgagagccccctactgcgctttggccacaccccctacgcctcgctcccggccccgcctctgcccctgaccgcgcctgcgcaaggcgggcgccctaaagtcctatttcactctgttgggaggagggggaaaggtgtacgcaggcgcagtggcgtctaaatttgggcccactaaatgcgtcggagcatctccgcgcccaggcggctcctcctcactgcggcaacccgggaaaacttgtgaactaatcagaaaaagtggaaggcgggagatcttggggcgctgtccaatggcgcggaagagaacaaatgagctggccaatcgggaacggcacgggggcgggctcgctcggcgcgaagttcgggcccgggaattccgaaggaggggtaggcgctgcccgcgcgcagaggccgcgcccctcctggccccggcttcttggctgtcaaacagatgcagcaacgtcggctcctgccgaggagcccaaggggtcccgggatccgccgcacaggctggcactgcttgaagaggaggctactcggagactgcgccgcgcgggtagatccgaaacggggctggggcggagtgggaaaaggccgggtatgccttgcatgatcgcggggagctccttcctgtttttatcccacctagagaagccgggaagtaggggtttaggtccaatttgttggagtacttaaggactcgtttgcactttcttttgggggatgacagtggattcattgccctcggaggttcaaccagttatgagtgagggattggccagaagatcggggcgcaggcaagcaggagtgctctattaggataagcaagtttgacaggaagaagctgctcttctccgaattacacagaggtgatgtgttcgtattgcacgtagacgtgtgtataacaggacctccttccccgcgccccgccaccccgacacacacaggagctgcctaaagtatccttgccttgcagattggaggctccccaaatattttgygatctgaggatccagctcaagtgaggtgccataggacgtgttcctgagtttgcattgcacggagaccttcctggaatttttcatttgcaagtyggcttaaccaattttgcattgagtcctaggctgcttgcactctgaatttgggctattcaggtagtgtgctcaaagttgaaaccgcatacagcacaactcaagtttgcatcagactgggaagcgaacttaagccagcggtgcgtggcccaggagtgggaaaggaaatggatgcctgaagtggaagaggtggtgcagagggggcaccgcccatgctgccctgcttccaactgctgcgcatagggggcggcaggggcggtgatctctacaccttccacccccccgccggggttggctgcacctatcgcttgggccacagggccgacctgtgtgatgtggccctgcggccccagcaggagcctggcctcatctctgggatccacgccgaactgcatgccgagccccggggtgatgactggagggtcagcctggaagaccacagcagccaaggtgagcattaagcagggcagctttgcccctgggtggttgaagcgccaggctggaatgagtaaggtctccacaagaccytgctgcctgcctcccatactcccatcagattggatggatagtcgtggtccagaccttcatcttcccaccagaagtgtgcacagtcagaagctctctgccagactgaccctttttggtcccgtttagctcatacaggacctgggatatcatcagaaagatatcacagtggggatgttctgaggccactagaggccaagtttagacttgattcagtttccagctttgctgaggcactctgttcctgggttagggcagttctatgttgaataatgtttttaataatctgggcatgtctttctccgtgacttgaggcagttagcctcagaaagcctagattcacatttgagttttgccactgcctcttggtaaagtcagctgtaggagtgttatggttattagactatagtagccaacattcatctagtgcttactgttatgagccaggccctattttaagtgtattgaatgtaggtggtactaatattatcctcatttacagtaaaggaaaatgaggcacaaagaggttaaggaacttgtccagggctgggcatggtggtttacacctataatccagcactttgggaggctaaggcagggtggatcacttgagctcaggagttcgagaccagcctgggcaacatggtgaaaacctgtctctaccaaaaaattaattaattttttaaaaaaagcctgggcgcggtggctcacgcctgtaatcccagcactttgggaggccgagatgggcagatcacgaggtcaggagttcgagaccatcctgaccaacatgttgaaaccccatctgtgctgaaaaaaaaatacaaaaattagccaggtgtggtggcgtgcacctgtaaccccagctactcaggaggctgaagcagcagaatcacttgaacccgggaggcggaggttgcagtgagctgagatcgcaccactgcactccagcttgggcgacagagcgagactccatctcaaacaaacaaacaaaccaaaagcttgcccagggtcacataactggtaagtggtagagctaggatctgaacgagctggagctgggggagagtgagcatgtttgaaaactggaccttagggcggggcacggtggctcacgcctgtaatcccagcactttgggaggctgaggcgggcagatcaggaggtcaggagtatgagaccagcctggccaacatggtaaaaccctgtctctgctaaaaataaaaaaattagccagacgtggtggcacatgcctgtaatcccagctactcaggaggctgaggcaggagaattgcttgaacctgggaggcggagtgcagtgagctgagattgcactactgcactccagcttgggcaatagagcaaaactccatctcaaaaaaaaaaaaaagaaagaaaaaaaaagaagaaagaaagaaaattggaccttaggacagtgagggcagggatcctttgtaggaaagcacaagaaacacagacttgttcctagctgacaaggagtgtactgcctggtacctgtcacctgctgaggggcttaggatgtgagggagaatctgactacagtttcatattcttccccagaaatcatacagatttctccactcctgactctggtcatttctgtttttgtcctccatatttgcctggtgccccaccatcaacaggtactttggtcaataatgtccgactcccaagaggtcacaggctggaattgagtgatggagacctcctgacctttggccctgaagggyccccaggaaccagcccctcggagttctacttcatgttccaacaagtacgagtcaagcctcaggactttgctgccattaccatcccacggtctaggggagaagcccgggttggggctggtttccggcctatgctgccctcccagggggctccacagcggcctctcagcaccttctcccctgcccccaaggccacactgatcctaaactccataggcagcctcagcaagctccggccccagcccctcaccttctcccctagttggggtggaccaaagagcctgcctgttcccgccccacctggggaartggggaccacgccttctgctccaccccaacgcaatcggaggaaatctgttcaccgagtgttggcggaactggatgatgagagtgagcctcytgagaacccgccaccggtccttatggagcccaggaagaaactccgtgtagacaaagccccactgactcccactgggtaagtggagtcctcacttggccctctcagtgttttactgcttttcgattccttgtatccctaggctgtgaggaggtccccctgcctggggggatgggcacgggaggtggaatagatggaatggcaagacctgggttagctctgataggaaaagaaaaatatgtgcaggagaacatgagaggtggggtggggcagtgcttataaaacaaccggagtgagcatgtcctgctttttacattcatatggctttaaccccattcttctagtgcctaaggatggggaactttcaggctcacactagaggtttttaggcccaccctatgtgtttttaaggacagagtccaggctcaccttagttctcagaccactgtgcctctgtggcctcaccctatgaccagccatagggtggcaaggtctaggccttctcctacaggtttccggtgacccttgtgtctgtgtcacttccttcagaaatcgacgtggccgtcctcggaagtacccagtgagcgctcccatggctccccctgcagttggggcggggagccctgtgcagctccttgttgctgcctgccccaggaagagacagtggcctgggttcagtgtgatggctgtgacgtctggttccatgtggcctgtgttggctgcagcatccaggctgccagggaggccgacttccgatgcccagggtgccgggctggcattcagcctaaggtccaccgccaaggcaccatcggacacacctgcccatgagtagacacagcagcgagcaaataggtctgataaataccccccttcccttccctccccargagggaatgactacagggaagaaggatggattgatgtggactcattcagggcctggagcagaccctggtggccaagacagaagagatggtttcctgccaaagatattgccacctccaggaaattgccagtgagctggaagttcccactattacaagccataaggccatgttgccatggacaccagaatatctgtagtcagagcacctatcagttgcaaaagccatgcctgcaaccgatggaaaatgtaagagggagttcttaaggttcttggtggcatcacccaaggcattctgggaaaacctagggcctggccccaaaacttccctactctgtggctagtcctgctgccaacaaaatcgtagcgacctggcttttcacagctttgcttttatttccaagtcaaggacaagccgcttcattcactcctgggcatttactcttcttgtgggtctgtgatattccttgctttccagggagaatgtgcttggcaaggtctggagaactaattcagaatcttaggggaaggggagagatggaaatacaaacctgcttactggaaaggtgcaaatatatgggttgagctggaggtaggaatacaggtaattaaggtttctagtttaagggaaaacagatctattgccatttaaataaggtaactgggatttggttaagttcacaaagatagcagaagatttatttacaggcttcacctgtactgtcagggcaagagaaagcctggtaaaccagctacagcagtttaccagtgtgatggctgtgacacagctccactccacgggtggacacagcagagggcaactgggctggcctggttcagtgtgaatcaaaccgcttaacccacacatggtacatgtgattttcttttgtgagccttacaccaagccaaactattgtcaaagcatcatttctatagaaataaagccttatcttgacctgttctattaaaacctgccacatccgccctttcctacctagattta

(19) United States
(12) Patent Application Publication
(10) Pub. No.: US 2008/0020383 A1
(43) Pub. Date: Jan. 24, 2008

(54) Haplotype Markers And Methods Of Using The Same To Determine Response To Treatment

(75) Inventors: Beena Koshy, Chapel Hill, NC (US); Bradley J. Dain, Hamden, CT (US); Madan Kumar Anant, Cheshire, CT (US); Ping Zhan, Rockville, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: GENAISSANCE PHARMACEUTICALS, Inc., New Haven, CT (US)

(21) Appl. No.: 11/578,342

(22) PCT Filed: May 4, 2005
(86) PCT No.: PCT/US05/15531

§ 371 Date: Aug. 29, 2007

Related U.S. Application Data
(60) Provisional application No. 60/568,371, filed on May 4, 2004.

(51) Int. Cl. C12Q 1/68; C07H 21/00

(52) U.S. Cl. 435/6; 536/23.1

(57) ABSTRACT

The present invention relates to methods for diagnosing or predicting responsiveness to treatment, such as Alefacept, by determining the presence of a nucleotide at one or more polymorphic sites within a haplotype marker. The present invention identifies multiple haplotypes that are associated with response to Alefacept. The haplotype markers identified by the present invention and methods of the invention can be particularly useful for diagnosing or predicting susceptibility to skin diseases, such as Psoriasis.

### BACKGROUND OF THE INVENTION

Variations or mutations in DNA are directly related to almost all human phenotypic traits and diseases. The most common type of DNA variation is a single nucleotide polymorphism (SNP), which is a base pair substitution at a single position in the genome. It has been estimated that SNPs account for the bulk of the DNA sequence difference between humans (Patil, N. et al., Science, 294:1719 (2001)). Blocks of such SNPs in close physical proximity in the genome are often genetically linked, resulting in reduced genetic variability within the population and defining a limited number of “SNP haplotypes”, each of which reflects descent from a single, ancient chromosome (Stephens, J. C., Molec. Diag. 4(4):309-317; Fullerton, S. M., et al., Am. J. Hum. Genet. 67: 881 (2000)).

Patterns of human DNA sequence variation, defined by SNPs, haplotypes or other types of variation, have important implications for identifying associations between phenotypic traits and genetic loci. For example, specific genomic regions of interest may be further analyzed to associate SNPs or haplotypes with phenotypic traits—e.g., disease susceptibility or resistance, a predisposition to a genetic disorder, or drug response. This information may be invaluable in understanding the biological basis for the trait as well as in identifying candidate genes useful in the development of therapeutics and diagnostics.

Psoriasis is one of the most common dermatologic diseases (also referred to herein as skin diseases), affecting up to 1 to 2% of the world's population. It is a chronic inflammatory skin disorder clinically characterized by erythematous, sharply demarcated papules and rounded plaques, covered by silvery micaceous scale. Traumatized areas often develop lesions of psoriasis (Koebner or isomorphic phenomenon). Additionally, other external factors may exacerbate psoriasis including infections, stress, and medications. About 5 to 10% of patients with psoriasis have associated joint complaints, and these are most often found in patients with fingernail involvement. Although some have the coincident occurrence of classic rheumatoid arthritis, many have joint disease.

The etiology of psoriasis is still poorly understood. There is clearly a genetic component to psoriasis. Over 50% of patients with psoriasis report a positive family history, and a 65 to 72% concordance among monozygotic twins has been reported in twin studies. Psoriasis has been linked to HLA-Cw6 and, to a lesser extent, to HLA-DR7. Evidence has accumulated clearly indicating a role for T cells in the pathophysiology of psoriasis. Stimulation of immune function with cytokines, such as IL-2, has been associated with abrupt worsening of pre-existing psoriasis, and bone marrow transplantation has resulted in clearance of disease. Psoriatic lesions are characterized by infiltration of skin with activated memory T cells, with CD8+ cells predominating in the epidermis. Agents that inhibit activated T cell function are often effective for the treatment of severe psoriasis.

Treatment of psoriasis depends on the type, location, and extent of disease. Most patients with localized plaque-type psoriasis can be managed with mid-potency topical glucocorticoids, although their long-term use is often accompanied by loss of effectiveness. Crude coal tar (1 to 5% in an ointment base) is an old but useful method of treatment in conjunction with ultraviolet light therapy. A topical vitamin D analogue (calcipitriol) is also efficacious in the treatment of psoriasis. Methotrexate is an effective agent, especially in patients with associated psoriatic arthritis. Liver toxicity from long-term use limits its use to patients with widespread disease not responsive to less aggressive modalities. The synthetic retinoid, acetretin, has been shown to be effective in some patients with severe psoriasis but is a potent teratogen, thus limiting its use in women with childbearing potential.

Despite the many treatments available for psoriasis, there is currently no reliable method for predicting the response of a subject to treatment for a skin disease, such as psoriasis. Thus, there is a need in the art for a reliable, non-invasive method for predicting the responsiveness of a subject to a treatment for a skin disease, such as psoriasis.

### SUMMARY OF THE INVENTION

The present invention is based, at least in part, on the discovery of genetic polymorphisms in select genes, including genes involved in T-cell activation and inhibition, e.g., CD8B1, HCR, SPR1, and TCF19, which are associated with an individual's response to a treatment, such as treatment with a T cell depleting agent, e.g., Amevive™ (also known as “Alefacept”).

Accordingly, the present invention provides a method of determining a subject's responsiveness to a treatment, such as treatment with Alefacept. In some embodiments, the method includes determining the nucleotide present at one or more polymorphic sites within a T cell activation or inhibition haplotype in a sample derived from a subject. In other embodiments, the method includes analyzing a sample derived from said subject to determine the subject's copy number for a T cell activation or inhibition haplotype. The T cell activation or inhibition haplotype may, for example, be a haplotype in any of the genes set forth in Table 1, e.g., a haplotype in the CD8B1, SPR1, TCF19, or HCR gene.

The invention further provides a method of determining a subject's responsiveness to a treatment by determining the genotype, e.g., the nucleotide present at one or more polymorphic sites on one or more chromosomes, within a T cell activation or inhibition haplotype in a sample derived from a subject. The T cell activation or inhibition haplotype may be one for which the p-value for the association of the haplotype to the subject's responsiveness to treatment indicates a high level of significance. In one embodiment, the T cell activation or inhibition haplotype is a haplotype in the CD8B1 gene, wherein the p-value for the association between the haplotype and responsiveness to treatment is less than or equal to about 0.005. In another embodiment, the T cell activation or inhibition haplotype is a haplotype in the SPR1 gene, wherein the p-value for the association between the haplotype and responsiveness to treatment is less than or equal to about 0.005. In still another embodiment, the T cell activation or: inhibition haplotype is a haplotype in the TCF19 gene, wherein the p-value for the association between the haplotype and responsiveness to treatment is less than or equal to about 0.010. In a further embodiment, the T cell activation or inhibition haplotype is a haplotype in the HCR gene and wherein the p-value for the association between the haplotype and responsiveness to treatment is less than or equal to about 0.007. In some embodiments, the method may further include determining the copy number of the T cell activation or inhibition haplotype using the subject's genotype determined at one or more polymorphic sites in the haplotype.

In one aspect, the invention provides a kit comprising an oligonucleotide selected from the group consisting of one or more oligonucleotides suitable for genotyping an SNP in a T cell activation or inhibition haplotype in the CD8B1, HCR, SPR1, and TCF19 genes, whereby the copy number of the T cell activation or inhibition haplotype provides a statistically significant association with whether a group of subjects suffering from a T cell associated disease, e.g., psoriasis, will respond or not respond to a T cell depleting agent, such as Alefacept. In one embodiment, the association between the subject's response and the T cell activation or inhibition haplotype is determined by a raw p-value, e.g., a raw p-value of less than or equal to about 0.005 for the CD8B1 gene; a raw p-value of less than or equal to about 0.007 for the HCR gene; a raw p-value of less than or equal to about 0.005 for the SPR1 gene; and a raw p-value of less than or equal to about 0.010 for the TCF19 gene.

In yet another aspect, the invention provides a kit for detecting the presence of a T cell activation or inhibition haplotype correlated with response or nonresponse to a T-cell depleting agent, such as Alefacept, the kit comprising a set of oligonucleotides designed for genotyping the polymorphic sites within the T cell activation or inhibition haplotype, wherein the T cell activation or inhibition haplotype is a haplotype in a gene selected from the group consisting of CD8B1, HCR, SPR1, and TCF19. Thus, the haplotype may, for example, be haplotype marker 1-5 in Table 1 or a haplotype in Tables 3A, 3B, 7A, 7B, 12, 16A or 16B; a linked haplotype marker to any one of the haplotypes in Tables 1, 3A, 3B, 7A, 7B, 12, 16A or 16B; or a substitute haplotype marker for any one of the haplotypes in Tables 1, 3A, 3B, 7A, 7B, 12, 16A or 16B.

In still another aspect, the invention provides a kit comprising an oligonucleotide selected from the group consisting of one or more oligonucleotides suitable for genotyping an SNP in the CD8B1, HCR, SPR1, and TCF19 genes for diagnosing the response of a subject suffering from a disease to a treatment regime. The SNP may, for example, be selected from the polymorphisms at: positions −685, −255, 25, 8632, 15080, 19501, 28589, 28663 and 28739 in the CD8B1 gene, positions 2173, 2175, 2360, 5782, 5787, 6174, 6666, 8277, 8440, 8476, 11565, 11941, 12152, 13553, 13892, 14287 in the HCR gene, positions −119, −845, −455, −384, −228, 161, 627, 739, 913 and 1171 in the SPR1 gene, and −303, −210, 316, 2059, 2365, 2456 and 3340 in the TCF19 gene. In addition, the kit may further comprise instructions of use. In another embodiment, the oligonucleotide is capable of detectably hybridizing to the SNP.

In one embodiment, the invention comprises a single-stranded oligonucleotide suitable for genotyping an SNP in a T cell activation or inhibition haplotype in the CD8B 1, HCR, SPR1, or TCF19 genes. The SNP may, for example, be selected from the polymorphisms at: positions −685, −255, 25, 8632, 15080, 19501, 28589, 28663 and 28739 in the CD8B1 gene, positions 2173, 2175, 2360, 5782, 5787, 6174, 6666, 8277, 8440, 8476, 11565, 11941, 12152, 13553, 13892, 14287 in the HCR gene, positions −119, −845, −455, −384, −228, 161, 627, 739, 913 and 1171 in the SPR1 gene, and −303, −210, 316, 2059, 2365, 2456 and 3340 in the TCF19 gene.

In another aspect, the present invention provides a method of determining a subject's responsiveness to a T cell depleting agent. In some embodiments, the method includes determining the genotype at one or more polymorphic sites within a T cell activation or inhibition haplotype in a sample derived from a subject. In other embodiments, the method includes analyzing a sample derived from the subject to determine the presence or absence in the subject of a T cell activation or inhibition haplotype or to determine the subject's copy number for a T cell activation or inhibition haplotype. The haplotype may, for example, be haplotype marker 1-5 in Table 1 or a haplotype in Tables 3A, 3B, 7A, 7B, 12, 16A or 16B; a linked haplotype marker to any one of the haplotypes in Tables 1, 3A, 3B, 7A, 7B, 12, 16A or 16B; or a substitute haplotype marker for any one of the haplotypes in Tables 1, 3A, 3B, 7A, 7B, 12, 16A or 16B. The T cell depleting agent may, for example, be an LFA-3 related molecule, or a CD2 receptor blocking agent, e.g., Alefacept.

For example, according to the invention, the presence in a subject of a CD8B1 haplotype comprising cytosine at position −255, a thymine at position 25 and a guanine at position 28589 indicates unresponsiveness by the subject to Alefacept. Likewise, the presence in a subject of two copies of an HCR haplotype comprising cytosine at position 2175, a thymine at position 5787 and a guanine at position 11565 or an HCR haplotype comprising guanine at position 5782, a guanine at position 11565, and a cytosine at position 14287 indicates unresponsiveness by the subject to Alefacept. The presence in a subject of a SPR1 haplotype comprising guanine at position −845, a guanine at position −455 and an adenine at position 1171 indicates responsiveness by the subject to Alefacept. Moreover, the presence in a subject of a TCF19 haplotype comprising guanine at position 2365 and a guanine at position 3340 indicates unresponsiveness by the subject to Alefacept.

In one embodiment, determining the presence of a nucleotide in the sample may be achieved by allele specific oligonucleotide hybridization, sequencing, primer specific extension, or protein detection.

In another embodiment, linkage disequilibrium between the linked haplotype marker and the haplotype marker has a Δ² selected from the group consisting of at least 0.75, at least 0.80, at least 0.85, at least 0.90, at least 0.95, and 1.0. In a preferred embodiment, Δ² is at least 0.95.

In yet another aspect, the present invention provides a method for selecting an appropriate treatment regime, e.g., the administration of a pharmaceutical, such as Alefacept, for a subject suffering from a disease. The method includes determining the genotype of the subject at one or more polymorphic sites within a haplotype, including haplotype markers in Tables 1, 3A, 3B, 7A, 7B, 12, 16A or 16B, a linked haplotype marker to any one of the haplotypes in Tables 1, 3A, 3B, 7A, 7B, 12, 16A or 16B, or a substitute haplotype marker for any one of the haplotypes in Tables 1, 3A, 3B, 7A, 7B, 12, 16A or 16B, in a sample derived from the subject; and selecting an appropriate treatment regime for the subject based on the subject's genotype at one or more polymorphic sites within the haplotype. In some embodiments, the copy number of the haplotype present in the subject is determined from the subject's genotype at one or more polymorphic sites within the haplotype. Indeed, the methods described herein may be used to select a treatment regime for a disease, such as a disease associated with T cell activation or inhibition, a disease associated with a deleterious T cell response, an inflammatory disease, a skin disease, e.g., psoriasis or eczema.

Selection of an appropriate treatment regime for a subject suffering from a disease may be accomplished by collecting a sample from the subject; determining the presence of a nucleotide at one or more polymorphic sites within a haplotype, e.g., one of the haplotype “markers in Tables 1, 3A, 3B, 7A, 7B, 12, 16A or 16B; a linked haplotype marker to one of the haplotypes in Tables 1, 3A, 3B, 7A, 7B, 12, 16A or 16B; or a substitute haplotype marker for one of the haplotypes in Tables 1, 3A, 3B, 7A, 7B, 12, 16A or 16B, in a sample derived from the subject; and selecting an appropriate treatment regime for the subject based on the subject's genotype at one or more polymorphic sites within the haplotype.

The invention also features a method for determining the responsiveness of a subject suffering from a disease to a treatment regime by determining the genotype at one or more polymorphic sites within a haplotype, e.g., the haplotypes in Tables 1, 3A, 3B, 7A, 7B, 12, 16A or 16B, a linked haplotype marker to one of the haplotypes in Tables 1, 3A, 3B, 7A, 7B, 12, 16A or 16B, or a substitute haplotype marker for one of the haplotypes in Tables 1, 3A, 3B, 7A, 7B, 12, 16A or 16B, in a sample derived from the subject. In one embodiment of the invention, the disease is psoriasis and the treatment regime includes the administration of Alefacept.

In a further aspect, the present invention features a method for treating a subject suffering from a disease. The method includes determining the genotype of the subject at one or more polymorphic sites within a haplotype, e.g., the haplotypes in Tables 1, 3A, 3B, 7A, 7B, 12, 16A or 16B, a linked haplotype marker to one of the haplotypes in Tables 1, 3A, 3B, 7A, 7B, 12, 16A or 16B, or a substitute haplotype marker for one of the haplotypes in Tables 1, 3A, 3B, 7A, 7B, 12, 16A or 16B, in a sample derived from the subject; selecting an appropriate treatment regime for the subject based on the subject's genotype at one or more polymorphic sites within the haplotype; and administering the treatment regime to the subject. In one embodiment of the invention, the disease is psoriasis and the treatment regime includes the administration of Alefacept.

The invention provides a method for testing an individual for the presence of a haplotype correlated with a response or non-response to a T-cell depleting agent, comprising analyzing a sample derived from said subject to determine the subject's copy number for a T cell activation or inhibition haplotype. The T cell activation or inhibition haplotype may be any one of the CD8B1 haplotypes shown in Tables 3A and 3B, the HCR haplotypes shown in Tables 7A and 7B, the SPR1 haplotypes shown in Table 12 and the TCF19 haplotypes shown in Tables 16A and B, a linked haplotype to any one of the CD8B1 haplotypes shown in Tables 3A and 3B, the HCR haplotypes shown in Tables 7A and 7B, the SPR1 haplotypes shown in Table 12 and the TCF19 haplotypes shown in Tables 16A and B, or a substitute; haplotype for any one of the CD8B1 haplotypes shown-in Tables 3A and 3B, the HCR haplotypes shown in Tables 7A and 7B, the SPR1 haplotypes shown in Table 12 and the TCF19 haplotypes shown in Tables 16A and B.

In another aspect, the invention features a method for identifying a subject who is likely to be unresponsive or responsive to treatment with Alefacept by determining the subject's copy number for a T cell activation or inhibition haplotype in a sample derived from the subject. The T cell activation or inhibition haplotype may be any one of the CD8B1 haplotypes shown in Tables 3A and 3B, the HCR haplotypes shown in Tables 7A and 7B, the SPR1 haplotypes shown in Table 12 and the TCF19 haplotypes shown in Tables 16A and B; a linked haplotype to any one of the CD8B1 haplotypes shown in Tables 3A and 3B, the HCR haplotypes shown in Tables 7A and 7B, the SPR1 haplotypes shown in Table 12 and the TCF19 haplotypes shown in Tables 16A and B; or a substitute haplotype for any one of the CD8B1 haplotypes shown in Tables 3A and 3B, the HCR haplotypes shown in Tables 7A and 7B, the SPR1 haplotypes shown in Table 12 and the TCF19 haplotypes shown in Tables 16A and B.

Other features and advantages of the invention will be apparent from the following detailed description, and from the claims.

### BRIEF DESCRIPTION OF THE DRAWINGS

FIGS. 1-7 depict odds ratio (OR) plots indicating an association between a haplotype marker of the invention in a particular gene to Alefacept response. The legends of each Figure are as follows. Upper Panel: Alefacept treated group; Lower panel: Placebo treated group. OR values are shown on the Y-axis. The X-axis indicates the copy number of the marker, where numbers in parentheses are the number of subjects in each group. The number in the title refers to the position of the SNP relative to the initiator ATG in the genomic sequence. SNP numbers in parentheses refer to the position of the SNP on the genomic structure using the initiation codon (ATG) as the reference. The p-values were calculated based on (1) a dominant or recessive model or (2) models comparing 0, 1, or 2 copies of the marker. Points are ORs of each group using the reference group indicated on the x-axis. Lines are 95% confidence interval of the ORs. Numbers above each line are ORs.

FIG. 1 depicts an OR plot indicating association of haplotype marker 1 (−255;25;28589/CTG) in the CD8B1 gene to Alefacept response.

FIG. 2 depicts an OR plot of the CD8B1 significant marker using a dominant model of inheritance.

FIG. 3 depicts an OR plot indicating association of haplotype marker 2 (2175,5787,11565/CTG) in the HCR gene to Alefacept response.

FIG. 4 depicts an OR plot of the HCR significant marker using a recessive model of inheritance.

FIG. 5 depicts an OR plot indicating association of haplotype marker 3 (−845, −455, 1171/GGA) in the SPR1 gene to Alefacept response.

FIG. 6 depicts an OR plot of the SPR1 significant marker using a dominant model of inheritance.

FIG. 7 depicts an OR plot indicating association of haplotype marker 4 (2365,3340/GG) in the TCF19 gene to response to Alefacept.

### DETAILED DESCRIPTION OF THE INVENTION

I. Definitions

So that the invention may be more readily understood, certain terms are first defined.

As used in the specification, “a” or “an” means one or more. As used in the claim(s), when used in conjunction with the word “comprising”, the words “a” or “an” mean one or more. As used herein, “another” means at least a second or more.

“Gene” is intended to mean the ORF (open reading frame) encoding an RNA or polypeptide, intronic regions, and the adjacent 5′ and 3′ non-coding nucleotide sequences, which may extend up to about 10 kb beyond the coding region, but possibly further in either direction. The adjacent and intronic sequences may be involved in the regulation of expression of the encoded RNA or polypeptide.

An “isogene” as used herein, refers to one of the isoforms of a gene found in a population. An isogene contains all of the polymorphisms present in the particular isoform of the gene.

The “odds ratio” or “OR” as used herein, is a way of comparing the probability of being a responder or non-responder to a T cell depleting agent, e.g., Alefacept, given the presence or absence of particular copy numbers of certain genetic HAP markers. The OR can be interpreted as a measure of the magnitude of association between the copy number of the genetic HAP marker and strong or poor response to the a T cell depleting agent. The OR is derived from the assessment of associations between genetic HAP markers to the binary outcome of strong response or poor response to the T cell depleting agent using logistic regression analysis. For example, with respect to haplotype marker 1 of the CD8B1 gene, an OR for the drug Alefacept is 5.2. The ratio is derived by comparing individuals who possess 0 copies of haplotype marker 1, as opposed to those who may have 1 or 2 copies of this haplotype marker. As a result, the OR indicates that the odds of responding to Alefacept is 5.2 times more likely in subjects who have 0 copies of haplotype marker 1 in the CB8B1 gene as opposed to the subject who possess 1 or 2 copies of this haplotype marker.

As used herein, the “p-value” refers to the probability that a given result obtained in a statistical test could have occurred by chance alone rather than because of a hypothesized relationship. For example, if a correlation coefficient has p<0.05, we infer that the observed correlation is not likely to have been a random occurrence as the p-value suggests that particular correlation would be obtained by chance alone fewer than 5 times out of 100. The “raw p-value” for the marker refers to the p-value of the association between the haplotype marker and the endpoint, adjusted for the covariates in the logistic regression but not for multiple comparisons. As described in the Examples, a “permutation adjusted p-value” further adjusts the raw p-value for multiple comparisons using a permutation test. It will be appreciated that 0.05 significance level is commonly used in the art. For example, if p<0.05 then results are very highly significant (Rosner B, 1990. Fundamentals of Biostatistics, 3rd Edition. PWS-Kent Publishing Company. Boston, Mass).

“Polymorphism” refers to a genetic variation, or the occurrence of two or more genetically determined alternative sequences or alleles at a single genetic locus in a population. Polymorphisms may have two alleles, with the minor allele occurring at a frequency of greater than 1%, and more preferably greater than 10% or 20% of a selected population. The allelic form occurring most frequently in a selected population is sometimes referenced as the “wildtype” form. Diploid organisms may be homozygous or heterozygous for allelic forms. A biallelic polymorphism has two forms. A triallelic polymorphism has three forms. Examples of polymorphisms include restriction fragment length polymorphisms (RFLPs), variable number of tandem repeats (VNTRs), single nucleotide polymorphisms (SNPs), dinucleotide repeats, trinucleotide repeats, tetranucleotide repeats, simple sequence repeats, and insertion elements such as Alu. A “polymorphic site” refers to the position in a nucleic acid sequence at which a polymorphism occurs. A polymorphic site may be as small as one base pair.

An “SNP” or “single nucleotide polymorphism” is a polymorphism that occurs at a polymorphic site occupied by a single nucleotide. The site of the SNP is usually preceded by and followed by highly conserved sequences (e.g., sequences that vary in less than 1/100 or 1/1000 members of a population). As used herein, “SNPs” is the plural of SNP. SNPs are most frequently biallelic. A most common allele of an SNP is called a “major allele” and an alternative allele of such an SNP is called a “minor allele.” An SNP usually arises due to substitution of one nucleotide for another at the polymorphic site. A transition is the replacement of one purine by another purine or one pyrimidine by another pyrimidine. A transversion is the replacement of a purine by a pyrimidine or vice versa. SNPs can also arise from a deletion of a nucleotide or an insertion of a nucleotide relative to a reference allele. An “SNP location” or “SNP locus” is a polymorphic site at which an SNP occurs.

“Haplotype marker”, “HAP marker”, or “haplotype” refers to the combination of alleles at a set of polymorphisms in a nucleic acid sequence of interest. In particular, the present invention provides, at least in part, the haplotype markers set forth in Tables 1, 3A, 3B, 7A, 7B, 12, 16A and 16B. The haplotype markers of the invention are labeled based on the location of the contributing single nucleotide polymorphisms (SNPs) in the gene using the initiation codon (ATG) of the reference mRNA used herein for the gene as the reference for the +1 position. The notation used gives the ATG offset for the SNPs (5′ to 3′) followed by the allele at each position. For example, in the haplotype marker (−255,25,28589/CTG), also referred to herein as haplotype marker 1, in the CD8B1 gene, C is the allele at a promoter SNP at −255, and T and G are the alleles at exonic SNPs at positions 25 and 28589, respectively, in the gene. Haplotype markers 2-5 in Table 1 are similarly defined.

A “substitute haplotype” includes a polymorphic sequence that is similar to that of any one of haplotype markers 1-5 shown in Table 1 or haplotypes in Tables 3A, 3B, 7A, 7B, 12, 16A and 16B, but in which the allele at one or more of the specifically identified polymorphic sites in that haplotype marker has been substituted with the allele at a polymorphic site in high linkage disequilibrium with the allele at the specifically identified polymorphic site. A substitute haplotype is further described below.

A “linked haplotype” includes a haplotype that is in high linkage disequilibrium with any one of haplotype markers 1-5 shown in Table 1 or haplotypes in Tables 3A, 3B, 7A, 7B, 12, 16A and 16B. A linked haplotype may comprise other types of variation including an indel. A linked haplotype is further,described below.

As used herein, the term “T cell activation or inhibition haplotype” is intended to include any haplotype that is associated with T cell activation or T cell inhibition. A T cell activation or inhibition haplotype may be a haplotype in a gene that encodes a protein that is part of a cellular pathway that leads to T cell activation or T cell inhibition. For example, a T cell activation or inhibition haplotype is a haplotype in a T cell receptor gene, a co-receptor gene, an integrin gene or a gene associated with T cell recognition by natural killer (NK) cells. In preferred embodiments, a T cell activation or inhibition haplotype is a haplotype in the CD8B1, HCR, SPR1, or TCF19 gene (e.g., one of the haplotypes set forth in Tables 1, 3A, 3B, 7A, 7B, 12, 16A or 16B).

As used herein, the term “T cell depleting agent” is intended to include any agent that is capable of reducing T lymphocyte, e.g., CD4+, CD8+ or CD2+ T lymphocyte, levels in a subject. The T cell depleting agents encompassed by the present invention may reduce T lymphocyte levels by inhibiting the LFA-3/CD2 interaction. For example, a T cell depleting agent may reduce T lymphocyte levels by binding to CD2 and inhibiting the interaction between LFA-3 on antigen-presenting cells and CD2 on T lymphocytes. In a preferred embodiment, a T cell depleting agent is a CD2 binding molecule, such as a molecule containing the CD2 binding portion of the LFA-3 molecule. In an even more preferred embodiment, the T cell depleting agent is Alefacept.

“Linkage” or “linked” describes or relates to the tendency of genes, alleles, loci or genetic markers to be inherited together from generation to generation as a result of the proximity of their locations on the same chromosome; e.g., genetic loci that are inherited non-randomly.

“Linkage disequilibrium” or “allelic association” includes the preferential association of a particular allele or genetic marker with a specific allele or genetic marker at a nearby chromosomal location more frequently than expected by chance for any particular allele frequency in the population. For example, if locus X has alleles a and b, which occur with equal frequency, and linked locus Y has alleles c and d, which occur with equal frequency, one would expect the combination ac to occur with a frequency of 0.25. If ac occurs more frequently, then alleles a and c are in linkage disequilibrium. Linkage disequilibrium may result from natural selection of a certain combination of alleles or because an allele has been introduced into a population too recently to have reached equilibrium with linked alleles. A marker in linkage disequilibrium with another causative marker for a disease (or other phenotype) can be useful in detecting susceptibility to the disease (or other phenotype) notwithstanding that the marker does not cause the disease. For example, a marker (X) that is not itself a causative element of a disease, but which is in linkage disequilibrium with an isoform of a gene (including regulatory sequences) (Y) that is a causative element of a phenotype, can be used to indicate susceptibility to the disease in circumstances in which the gene Y may not have been identified or may not be readily detectable.

“Nucleic acids” include, but are not limited to, DNA, RNA, single- or double-stranded, genomic, cloned, naturally occurring or synthetic molecules and may be polynucleotides, amplicons, RNA transcripts, protein nucleic acids, nucleic acid mimetics, and the like.

“Oligonucleotides” are well known in the art and include nucleic acids that are usually between 5 and 100 contiguous bases in length, and often between 5-10, 5-20, 10-20, 10-50, 15-50, 15-100, 20-50, or 20-100 contiguous bases in length. An oligonucleotide that is longer than about 20 contiguous bases may be referred to as a polynucleotide. A polymorphic site (polymorphism) can occur at any position within an oligonucleotide. An oligonucleotide may include any of the allelic forms of the polymorphic sites (polymorphisms). Other oligonucleotides useful in practicing the invention hybridize to a target region located one to less than or equal to about 10 nucleotides adjacent to a polymorphic site, preferably ≦about 5 nucleotides. Such oligonucleotides terminating one to several nucleotides adjacent to a polymorphic site are useful in polymerase-mediated primer extension methods for detecting one of the polymorphisms described herein and therefore such oligonucleotides are referred to herein as “primer-extension oligonucleotides”. In a preferred embodiment, the 3′-terminus of a primer-extension oligonucleotide is a deoxynucleotide complementary to the nucleotide located immediately adjacent to the polymorphic site.

“Hybridization probes” or “probes” are oligonucleotides capable of binding in a base-specific manner to a partially or completely complementary strand of nucleic acid. Such probes include peptide nucleic acids, as described in Nielsen et al., Science 254: 1497-1500 (1991), as well as all other kinds of oligonucleotides.

“Global assessment score” refers to a 7 point scale used to measure the severity of psoriasis at the time of the physician's evaluation: severe: very marked plaque elevation, scaling and/or erythema; moderate to severe: marked plaque elevation, scaling and/or erythema; moderate: moderate plaque elevation, scaling and/or erythema; mild to moderate: intermediate between moderate and mild; mild: slight plaque elevation, scaling and/or erythema almost clear: intermediate between mild and clear; clear: no signs of psoriasis (post-inflamatory hypopigmentation or hyperpigmentation could be present).

Hybridizations are usually performed under stringent conditions. Stringent conditions are sequence-dependent and vary depending on the circumstances. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. As the target sequences are generally present in excess, at Tm, 50% of the probes are occupied at equilibrium. Typically, stringent conditions include a salt concentration of at least about 0.01 to 1.0 M Na ion concentration (or other salts) at pH 7.0 to 8.3 and the temperature is at least about 25° C. for short probes (e.g., 10 to 50 nucleotides). Stringent conditions can also be achieved with the addition of destabilizing agents such as formamide. For example, conditions of 5× SSPE (750 mM NaCl, 50 mM NaPhosphate, 5 mM EDTA, pH 7.4) and a temperature of 25-30° C. are suitable for allele-specific probe hybridizations.

Additional stringent conditions can be found in Molecular Cloning: A Laboratory Manual, Sambrook et al., Cold Spring Harbor Press, Cold Spring Harbor, N.Y. (1989), chapters 7, 9, and 11. A preferred, non-limiting example of stringent hybridization conditions includes hybridization in 4× sodium chloride/sodium citrate (SSC), at about 65-70° C. (or alternatively hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or alternatively hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C. (see e.g., Church and Gilbert (1984) Proc. Natl. Acad. Sci. USA 81:1991-1995), or alternatively 0.2×SSC, 1% SDS.

A “Psoriasis Area and Severity Index” (PASI) score refers to a measurement of the severity of psoriasis (see e.g., Fleischer et al. (1999), J. Dermatol. 26:210-215 and Tanew et al. (1999), Arch Dermatol. 135:519-524). The PASI score is a measure of the location, size and degree of skin scaling in psoriatic lesions on the body. The PASI is a commonly-used measure in clinical trials for psoriasis treatment. Typically, the PASI is calculated before, during, and after a treatment period in order to determine how well psoriasis responds to treatment (e.g., a lower PASI means less psoriasis). For the PASI, the body is typically divided into four sections and each area is scored by itself, and then the four scores are combined into the final PASI. For each skin section, the amount of skin involved is measured as a percentage of the skin in that body section. The severity, e.g., itching, erythema (redness), scaling, and thickness are also measured for each skin section.

A “Strong responder” refers to a patient's response of greater than or equal to 50% reduction of PASI from baseline at any time. The term “Partial responder” refers a patient's response of greater than or equal to 25% but <50% reduction of PASI from baseline at any time. The term “Non-responders” refers a patient's response of less than 25% reduction of PASI from baseline at any time.

As used herein, the term “subject” includes warm-blooded animals, preferably mammals, including humans. In a preferred embodiment, the subject is a primate. In an even more preferred embodiment, the primate is a human.

As used herein, the term “therapeutically effective amount” refers to that amount of a therapeutic agent sufficient to result in amelioration of one or more symptoms of a disorder. With respect to the treatment of psoriasis, a therapeutically effective amount preferably refers to the amount of a therapeutic agent that reduces a subject's (e.g., human's) PASI score by at least 20%, at least 35%, at least 30%, at least 40%, at least 45%, at least 50%, at least 55%, at least 60%, at least 65%, at least 70%, at least 75%, at least 80%, or at least 85%. Alternatively, with respect to the treatment of psoriasis, a therapeutically effective amount preferably refers to the amount of a therapeutic agent that improves a subject's (e.g., human's) global assessment score by at least 25%, at least 35%, at least 30%, at least 40%, at least 45%, at least 50%, at least 55%, at least 60%, at least 65%, at least 70%, at least 75%, at least 80%, at least 85%, at least 90%, or at least 95%. A therapeutically effective amount includes an amount effective, at dosages and for periods of time necessary, to achieve the desired result, e.g., sufficient to treat a subject suffering from a disease or disorder, such as an inflammatory disease, e.g., skin disease. A therapeutically effective amount of a compound, such as a T cell depleting agent, e.g., Alefacept, as defined herein may vary according to factors such as the disease state, age, and weight of the subject, and the ability of the compound to elicit a desired response in the subject. Dosage regimens may be adjusted to provide the optimum therapeutic response. A therapeutically effective amount is also one in which any toxic or detrimental effects (e.g., side effects) of the T cell depleting agent, e.g., Alefacept, are outweighed by the therapeutically beneficial effects.

A “prophylactically effective amount” refers to an amount effective, at dosages and for periods of time necessary, to achieve the desired prophylactic result, such as preventing or inhibiting an inflammatory disease, such as a skin disease, e.g., psoriasis, in a subject predisposed to such a disease. A prophylactically effective amount can be determined as described abovezfor the therapeutically effective amount. Typically, since a prophylactic,. dose is used in subjects prior to or at an earlier stage of inflammatory disease, the prophylactically effective amount will be less than the therapeutically effective amount.

II. General

The present invention is based on the identification of multiple haplotypes associated with responsiveness to treatment and provides novel methods for determining a subject's responsiveness to a treatment regime, e.g., treatment with a T cell depleting agent, such as Alefacept.

The present invention includes the use of any of the haplotype markers of the invention, including those set forth in Tables 1, 3A, 3B, 7A, 7B, 12, 16A or 16B, as well as polymorphisms, alleles, or markers in linkage disequilibrium with these markers, as a means for diagnosing a subject's response to a treatment regime, or as a means for designing an effective therapeutic regime that is specifically tailored to a subject. The present invention is particularly useful in the treatment of dermatologic diseases, such as psoriasis and eczema.

Polymorphisms and haplotypes of the present invention are set forth in Tables 1, 3A, 3B, 7A, 7B, 12, 16A and 16B and were identified as described herein.

TABLE 1

| Haplotype | Gene | | Sequence |
|---|---|---|---|
| Marker No. | SSymbol | Haplotype Marker * | Identification No. |
| 1 | CD8B1 | (−255, 25, 28589/CTG) | 1 |
| 2 | HCR | (2175, 5787, 11565/CTG) | 2 |
| 3 | SPR1 | (−845, −455, 1171/GGA) | 3 |
| 4 | TCF19 | (2365, 3340/GG) | 4 |
| 5 | HCR | (5782, 11565, 14287/GGC) | 2 |
| * The Haplotype Markers are labeled based on the location of the contributing SNPs in the | | | |
| # gene using the initiation codon (ATG) as the reference for the +1 position. The notation | | | |
| # used gives the ATG offset for the SNPs (5′ to 3′) followed by the allele at each | | | |
| # position. For example, in the haplotype marker (−255, 25, 28589/CTG), also referred to | | | |
| # herein as haplotype marker 1, in the CD8B1 gene, C is the allele at a promoter SNP | | | |
| # at −255, and T and G are the alleles at exonic SNPs at positions 25 and | | | |
| # 28589, respectively, in the gene. | | | |

CD8B1 Gene

The CD8 antigen, beta polypeptide 1 (CD8B1) gene encodes the β subunit of the CD8 protein. The CD8β chain is a 34 kDa protein that consists of four discrete functional domains, the Ig-like ectodomain, the membrane proximal stalk (hinge) region, the transmembrane domain, and the cytoplasmic domain.

Human CD8 is a cell surface glycoprotein that is expressed on cytotoxic T cells and functions as a co-receptor along with the T cell antigen receptor (TCR). On mature peripheral class I MHC restricted T-cells, the CD8 molecule exists as a disulfide linked heterodimer of alpha and beta chains. However, the CD8β molecule can be expressed on the cell surface without the CD8 α chain. The extracellular domain can efficiently interact with the TCR/CD3 complex and is also capable of independent interaction with MHC class I/of β2 microglobulin dimers in the absence of CD8α. The cytoplasmic domain enhances and regulates the association with the intracellular signaling molecules necessary for effective signal transduction such as lymphocyte specific protein tyrosine kinase (LCK) and the linker of activation of T-cells (LAT).

The genomic sequence of CD8B1 is set forth herein as SEQ ID NO:1 and was derived from a combination of draft genomic sequences Accession Nos. AC111200.3 (GI:18873971 ) and AC112696.1 (GI:18860769) using reference mRNA X13445.1 to determine the ordering of the contigs.

HCR Gene

The a-helix coiled-coil rod homolog (HCR) gene is a gene that is part of the PSORS1 locus on chromosome 6. Genomic organization for HCR was provided by the annotation of Accession No. AB029343.1 (GI No.: 5360900) and is set forth herein as SEQ ID NO:2. Although the functional role of HCR is yet to be elucidated, the gene shows differential expression in normal and psoriatic skin. Two of the SNPs in the HCR gene, C2175 and T5787, result in amino acid changes that cause dramatic alternations in the secondary structure of the protein.

SPR1 Gene

Small proline-rich protein 1 (SPR1) is a gene that is part of the 300 kb region called PSORS1 around the HLA-C gene identified as being associated with susceptibility to psoriasis. The SPR1 gene lies about 3 kb telomeric to the HCR gene. The data indicate strong linkage disequilibrium between the two genes. The genomic sequence of SPR1 is set forth herein as SEQ ID NO:3 and was derived using the reference mRNA, NM_{—}014069.1 (GI No.: 7662664) and Genbank Accession No. AP000510.2 (GI No.: 7380878) as the genomic DNA.

TCF19 Gene

The Transcription Factor 19 gene (TCF19) is located on chromosome 6, between the HLA-C and the S genes, a region that has been implicated in pathophysiology of psoriasis vulgaris.

The TCF19 gene is expressed abundantly in different tissues. There are at least 10 different transcripts that are produced by alternative splicing, generating eight isoforms of the protein. The TCF19 protein has the Forkhead-associated (FHA) motif that is found in many regulatory proteins, such as kinases, phosphatases, transcription factors, and enzymes, which participate in many different cellular processes such as DNA repair, signal transduction, and protein degradation.

The genomic sequence of TCF19 was derived using the reference mRNA Genbank Accession No. NM007109.1 (GI No.:6005891) and DNA Accession No. AC004195.1 and is set forth herein as SEQ ID NO:4.

Additional Polymorphisms in Linkage Disequilibrium

For each haplotype marker 1 to 5 in Table 1 or in Tables 3A, 3B, 7A, 7B, 12, 16A and 16B, the present invention also includes other polymorphisms in that gene or elsewhere on the chromosome of that gene that are in high linkage disequilibrium (LD) with one or more of the polymorphisms comprising the haplotype marker. Two particular nucleotide alleles at different polymorphic sites are said to be in LD if the presence of one of the alleles at one of the sites tends to predict the presence of the other allele at the other site on the same chromosome (Stevens, J C, Mol. Diag. 4: 309-17, 1999). One of the most frequently used measures of linkage disequilibrium is Δ², which is calculated using the formula described in Devlin, B. and Risch, N. (1995, Genomics, 29(2):311-22). Basically, Δ² measures how well an allele X at a first polymorphic site predicts the occurrence of an allele Y at a second polymorphic site on the same chromosome. The measure only reaches 1.0 when the prediction is perfect (e.g., X if and only if Y).

Thus, the skilled artisan would expect that all of the embodiments of the invention described herein may frequently be practiced by substituting the allele at any (or all) of the specifically identified polymorphic sites in a haplotype marker disclosed herein with an allele at another polymorphic site that is in high LD with the allele at the specifically identified polymorphic site. This “substituting polymorphic site” may be one that is currently known or subsequently discovered and may be present at a polymorphic site in the same gene as the replaced polymorphic site or elsewhere on the same chromosome as the replaced polymorphic site. Preferably, the substituting polymorphic site is present in a genomic region within about 100 kilobases from the polymorphic site.

Further, for any particular haplotype presented in Table 1 or in Tables 3A, 3B, 7A, 7B, 12, 16A and 16B, the present invention contemplates that there will be other haplotypes in that gene or elsewhere on the same chromosome as that gene that are in high LD with one or more of the polymorphisms comprising the haplotype marker that would therefore also be predictive of the clinical phenotype (i.e., responsiveness to a treatment, e.g., treatment with Alefacept or age of onset of an inflammatory or skin disease). Preferably, the linked haplotype is present in the gene or in a genomic region of about 100 kilobases spanning the gene. The linkage disequilibrium between a disclosed haplotype marker and a linked haplotype can also be measured using Δ².

In preferred embodiments, the linkage disequilibrium between the allele at a polymorphic site in any of the disclosed haplotype markers and the allele at a substituting polymorphic site that may replace it, or between any of the disclosed haplotype markers and a linked haplotype, has a Δ² value, as measured in a suitable reference population, of at least 0.75, more preferably at least 0.80, even more preferably at least 0.85 or at least 0.90, yet more preferably at least 0.95, and most preferably 1.0. A suitable reference population for this Δ² measurement is preferably selected from a population for which the distribution of the ethnic background of its members reflects that of the population of patients to be treated with a treatment regime, e.g., Alefacept. The reference population may be the general population, a population using T cell depleting agents, e.g., Alefacept; a population suffering from an inflammatory disease or a skin disease, such as psoriasis; or a population with risk factors for developing an inflammatory or skin disease.

LD patterns in genomic regions are readily determined in appropriately chosen samples using various techniques known in the art for determining whether any two alleles (at two different polymorphic sites or two haplotypes) are in linkage disequilibrium (Weir B. S. 1996 Genetic Data Analysis II, Sinauer Associates, Inc. Publishers, Sunderland, Mass.). The skilled artisan may readily select which method of determining LD will be best suited for a particular sample size and genomic region. Similarly, the ability of substitute haplotypes, that contain an allele at one or more substituting polymorphic sites, or of linked haplotypes, that are in high LD with one or more of the haplotype markers in Tables 1, 3A, 3B, 7A, 7B, 12, 16A and 16B to predict the clinical response to a T cell depleting agent, e.g., Alefacept, may also be readily tested by the skilled artisan.

Thus, reference herein to a T cell activation or inhibition haplotype is deemed to include linked haplotypes to any disclosed haplotype and substitute haplotypes for any disclosed haplotype that behave similarly to the disclosed haplotype marker in terms of predicting a subject's clinical response to a T cell depleting agent, e.g., Alefacept.

III. Nucleic Acid Molecules Containing the Polymorphisms of the Present Invention

The invention is based, in part, on the discovery of polymorphisms and haplotype markers in the CD8B1, HCR, SPR1, and TCF19 genes (SEQ ID NOS:1-4). Thus, in one embodiment, the invention provides fragments of these genes (SEQ ID NOS:1-4) containing at least one single nucleotide polymorphism listed in Table 1.

An isolated polynucleotide containing a polymorphic variant nucleotide sequence (SNP) of the invention may be operably linked to one or more expression regulatory elements in a recombinant expression vector capable of being propagated and expressing the encoded variant proteins in a prokaryotic or a eukaryotic host cell. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce the variant proteins or peptides, encoded by nucleic acids as described herein.

The recombinant expression vectors of the invention can be designed for expression of proteins in prokaryotic or eukaryotic cells. For example, proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

IV. Methods of Use

The methods of the invention have utility in identifying polymorphisms and haplotype patterns in biological samples. This information may then be used in any number of ways including, but not limited to, selection of a treatment regime for a subject suffering from a disease, e.g., psoriasis; treatment efficacy and/or safety trials; genetic mapping of phenotypic traits (e.g., disease resistance or susceptibility, and drug response, including e.g., efficacy and adverse effects); diagnostics; identification of candidate drug targets; development of protein, small molecule, antisense, antibody, or other therapeutics; to reveal the biological basis for a phenotypic trait; association studies; forensics; and paternity testing.

A. Detection of Haplotype Markers of the Invention in Target Nucleic Acid Molecules

The polymorphisms and haplotype markers of the invention may be detected in a nucleic acid sample from a subject being screened, e.g., a subject undergoing treatment for a disease or a subject in need of treatment for a disease (e.g., psoriasis). Nucleic acid samples may be obtained from virtually any biological sample. For example, convenient samples include whole blood, serum, semen, saliva, tears, fecal matter, urine, sweat, buccal matter, skin and hair. For assays of cDNA or mRNA, the tissue should be obtained from an organ in which the target nucleic acid is expressed.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, N.Y.).

Nucleic acid samples may be prepared for analysis using any technique known to those skilled in the art. Preferably, such techniques result in the production of a nucleic acid molecule sufficiently pure for determining the presence or absence of one or more alleles at one or more locations in the nucleic acid molecule. Such techniques may be found, for example, in Sambrook, et al., Molecular Cloning: A Laboratory Manual (Cold Spring Harbor Laboratory, New York) (2001), incorporated herein by reference.

It may be desirable to amplify and/or label one or more nucleic acids of interest before determining the presence or absence of one or more alleles in the nucleic acid. Any amplification technique known to those of skill in the art may be used in conjunction with the present invention including, but not limited to, polymerase chain reaction (PCR) techniques. PCR may be carried out using materials and methods known to those of skill in the art (See generally PCR Technology: Principals and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Matilla et al., Nucleic Acids Res. 19: 4967 (1991); Eckert et al., PCR Methods and Applications 1: 17 (1991); PCR (eds. McPherson et al., IRL Press, Oxford); and U.S. Pat. No. 4,683,202, the entire contents of each of which are incorporated herein by reference). Other suitable amplification methods include the ligase chain reaction (LCR) (see Wu and Wallace, Genomics 4: 560 (1989) and Landegren et al., Science 241: 1077 (1988)), transcription amplification (Kwoh et al., Proc. Natl. Acad. Sci. USA 86: 1173 (1989)), self-sustained sequence replication (Guatelli et al., Proc. Nat. Acad. Sci. USA, 87: 1874 (1990)) and nucleic acid-based sequence amplification (NASBA).

Determination of the presence or absence of one or more alleles in a nucleic acid may be achieved using any technique known to those of skill in the art. Any technique that permits the accurate determination of a variation can be used. Preferred techniques permit rapid, accurate determination of multiple variations with a minimum of sample handling. Some examples of suitable techniques include, but are not limited to, direct DNA sequencing, capillary electrophoresis, hybridization, using, for example, allele-specific probes or primers, single-strand conformation polymorphism analysis, nucleic acid arrays, primer specific extension, protein detection, and other techniques well known in the art. Several methods for DNA sequencing are well known and generally available in the art and may be used to determine the allele present in a given individual. See, for example, Sambrook, et al., Molecular Cloning: A Laboratory Manual (Cold Spring Harbor Laboratory, New York) (2001), and Ausubel, et al., Current Protocols in Molecular Biology (John Wiley and Sons, New York) (1997), incorporated herein by reference. For details on the use of nucleic acid arrays (DNA chips) for the detection of, for example, SNPs, see U.S. Pat. No. 6,300,063 issued to Lipshultz, et al., and U.S. Pat. No. 5,837,832 to Chee, et al., HuSNP Mapping Assay, reagent kit and user manual, Affymetrix Part No. 90094 (Affymetrix, Santa Clara, Calif.), all incorporated herein by reference.

The detection methods of the invention can be used to detect the presence or absence of one or more alleles in a nucleic acid or polypeptide in a biological sample in vitro as well as in vivo. For example, in vitro techniques for detection of a nucleic acid molecule of interest include Northern hybridizations, Southern hybridizations and in situ hybridizations. In vitro techniques for detection of a polypeptide of interest include enzyme linked immunosorbent assays (ELISAs), Western blots, immunoprecipitations and immunofluorescence. Alternatively, the polypeptide can be detected in vivo in a subject by introducing into the subject a labeled antibody. For example, the antibody can be labeled with a radioactive marker whose presence and location in a subject can be detected by standard imaging techniques.

Southern or Northern analysis, dot blot, or other membrane based technologies, dipstick assays, and microarrays utilizing fluids or tissue extracts from patients may be used to detect the polymorphisms described herein. The polynucleotide sequences of the present invention, and longer or shorter sequences derived therefrom, may also be used as targets in a microarray, or other genotyping system. These systems can be used to detect the presence or absence of a large number of particular alleles or to monitor the expression of a large number of gene products simultaneously.

In a preferred embodiment, it is possible to use allele-specific probes to determine the genotype of the polymorphisms to determine the haplotype structure in a nucleic acid sample. The design and use of allele-specific probes for analyzing polymorphisms is described by, e.g., U.S. Pat. No. 6,361,947 issued to Dong, et al. Allele-specific probes can be designed that hybridize to a segment of target nucleic acid sample, e.g., DNA or RNA, from one individual but do not hybridize to the corresponding segment from another individual due to the presence of different polymorphic forms (alleles) in the respective segments from the two individuals. Hybridization conditions should be sufficiently stringent such that there is a significant difference in hybridization intensity between alleles, and preferably an essentially binary response, whereby a probe hybridizes to only one of the alleles. Some probes are designed to hybridize to a segment of target nucleic acid molecule such that the polymorphic site aligns with a central position (e.g., in a 15-mer at the 7^{th} position; in a 25-mer at the 13^{th} position) of the probe. This design of probe achieves good discrimination in hybridization between different allelic forms. In a preferred embodiment, a nucleic acid of the invention is specifically hybridized to a target nucleic acid as a means of detecting a polymorphism in the target nucleic acid. These allele-specific probes can also be immobilized on a nucleic acid array. An example of hybridization to a nucleic acid array involves the use of DNA chips (oligonucleotide arrays), for example, those available from Affymetrix, Inc. Santa Clara, Calif. In a preferred embodiment, nucleic acid arrays are used to detect the haplotype markers of the invention in a target DNA sample.

In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of polymorphism. See, for example, Cotton et al (1988) Proc. Natl Acad Sci USA 85:4397; and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an allele of a haplotype marker of the invention is hybridized to a cDNA or other DNA product from a test cell(s). The duplex is treated with a DNA mismatch repair enzyme, and the cleavage products, if any, can be detected from electrophoresis protocols or the like (See, for example, U.S. Pat. No. 5,459,039).

In yet another embodiment, the movement of alleles in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:12753).

For polymorphisms that produce premature termination of protein translation, the protein truncation test (PTT) offers an efficient diagnostic approach (Roest, et al., (1993) Hum. Mol Genet. 2:1719-21; van der Luijt, et al., (1994) Genomics 20:1-4). For PTT, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products.

The polymorphisms and haplotype markers of the invention can also be assessed by hybridization to nucleic acid arrays, some examples of which are described in WO 95/11995. WO 95/11995 also describes subarrays that are optimized for detection of a variant form of a precharacterized polymorphism. Such a subarray contains probes designed to be complementary to a second reference sequence, which is an allelic variant of the first reference sequence. The second group of probes is designed by the same principles, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to 21 bases).

Amplification products generated using the polymerase chain reaction can be analyzed by the use of denaturing gradient gel electrophoresis. Different alleles can be identified based on the different sequence-dependent melting properties and electrophoretic migration of DNA in solution (Erlich, ed., PCR Technology, Principles and Applications for DNA Amplification, W. H. Freeman and Co, New York, 1992, Chapter 7).

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence differences between alleles of target sequences.

An alternative method for identifying and analyzing polymorphisms is based on single-base extension (SBE) of a fluorescently-labeled primer coupled with fluorescence resonance energy transfer (FRET) between the label of the added base and the label of the primer. Typically, the method, such as that described by Chen et al., (Proc. Nat. Acad. Sci. 94:10756-61 (1997)) uses a locus-specific oligonucleotide primer labeled on the 5′ terminus with 5-carboxyfluorescein (FAM). This labeled primer is designed so that the 3′ end is immediately adjacent to the polymorphic site of interest. The labeled primer is hybridized to the locus, and single base extension of the labeled primer is performed with fluorescently labeled dideoxyribonucleotides (ddNTPs) in dye-terminator sequencing fashion, except that no deoxyribonucleotides are present. An increase in fluorescence of the added ddNTP in response to excitation at the wavelength of the labeled primer is used to infer the identity of the added nucleotide.

The presence in an individual of a haplotype marker X may be determined by a variety of indirect or direct methods well known in the art for determining haplotypes or haplotype pairs for a set of polymorphic sites in one or both copies of the individual's genome, including those discussed below. The genotype for a polymorphic site in an individual may be determined by methods known in the art or as described below.

One indirect method for determining whether zero or at least one copy of a haplotype is present in an individual is by prediction based on the individual's genotype determined at one or more of the polymorphic sites (PS) comprising the haplotype and using the determined genotype at each site to determine the haplotypes present in the individual. The presence of zero, one or two copies of a haplotype of interest can be determined by visual inspection of the alleles at the PS that comprise the haplotype. The haplotype pair is assigned by comparing the individual's genotype with the genotypes at the same set of PS corresponding to the haplotype pairs known to exist in the general population or in a specific population group or to the haplotype pairs that are theoretically possible based on the alternative alleles possible at each PS, and determining which haplotype pair is most likely to exist in the individual.

In a related indirect haplotyping method, the presence in an individual of zero copy or at least one copy of a haplotype is predicted from the individual's genotype for a set of PS comprising the selected haplotype using information on haplotype pairs known to exist in a reference population. In one embodiment, this haplotype pair prediction method comprises identifying a genotype for the individual at the set of polymorphic sites comprising the selected haplotype, accessing data containing haplotype pairs identified in a reference population for a set of polymorphic sites comprising the polymorphic sites of the selected haplotype, and assigning to the individual a haplotype pair that is consistent with the individual's genotype. Whether the individual has a haplotype marker X can be subsequently determined based on the assigned haplotype pair. The haplotype pair can be assigned by comparing the individual's genotype with the genotypes corresponding to the haplotype pairs known to exist in the general population or in a specific population group, and determining which haplotype pair is consistent with the genotype of the individual. In some embodiments, the comparing step may be performed by visual inspection. When the genotype of the individual is consistent with more than one haplotype pair, frequency data may be used to determine which of these haplotype pairs is most likely to be present in the individual. If a particular haplotype pair consistent with the genotype of the individual is more frequent in the reference population than others consistent with the genotype, then that haplotype pair with the highest frequency is the most likely to be present in the individual. This determination may also be performed in some embodiments by visual inspection. In other embodiments, the comparison may be made by a computer-implemented algorithm with the genotype of the individual and the reference haplotype data stored in computer-readable formats. For example, as described in WO 01/80156, one computer-implemented algorithm to perform this comparison entails enumerating all possible haplotype pairs which are consistent with the genotype, accessing data containing haplotype pair frequency data determined in a reference population to determine a probability that the individual has a possible haplotype pair, and analyzing the determined probabilities to assign a haplotype pair to the individual.

Typically, the reference population is composed of randomly-selected individuals representing the major ethnogeographic groups of the world. A preferred reference population for use in the methods of the present invention consists of Caucasian individuals, the number of which is chosen based on how rare a haplotype is that one wants to be guaranteed to see. For example, if one wants to have a q % chance of not missing a haplotype that exists in the population at a p % frequency of occurring in the reference population, the number of individuals (n) who must be sampled is given by 2n=log(1−q)/log(1−p) where p and q are expressed as fractions. A particularly preferred reference population includes a 3-generation Caucasian family to serve as a control for checking quality of haplotyping procedures.

If the reference population comprises more than one ethnogeographic group, the frequency data for each group is examined to determine whether it is consistent with Hardy-Weinberg equilibrium. Hardy-Weinberg equilibrium (D. L. Hartl et al., Principles of Population Genomics, Sinauer Associates (Sunderland, Mass.), 3^{rd} Ed., 1997) postulates that the frequency of finding the haplotype pair H₁/H₂ is equal to
p_{H-W}(H₁/H₂)=2p(H₁)p(H₂) if H₁≠H₂ and p_{H-W}(H₁/H₂)=p(H₁)p(H₂) if H₁=H₂.
A statistically significant difference between the observed and expected haplotype frequencies could be due to one or more factors including significant inbreeding in the population group, strong selective pressure on the gene, sampling bias, and/or errors in the genotyping process. If large deviations from Hardy-Weinberg equilibrium are observed in an ethnogeographic group, the number of individuals in that group can be increased to see if the deviation is due to a sampling bias. If a larger sample size does not reduce the difference between observed and expected haplotype pair frequencies, then one may wish to consider haplotyping the individual using a direct haplotyping method such as, for example, CLASPER System™ technology (U.S. Pat. No. 5,866,404), single molecule dilution, or allele-specific long-range PCR (Michalotos-Beloin et al., Nucleic Acids Res. 24:4841-4843, 1996).

In one embodiment of this method for predicting a haplotype pair for an individual, the assigning step involves performing the following analysis. First, each of the possible haplotype pairs is compared to the haplotype pairs in the reference population. Generally, only one of the haplotype pairs in the reference population matches a possible haplotype pair and that pair is assigned to the individual. Occasionally, only one haplotype represented in the reference haplotype pairs is consistent with a possible haplotype pair for an individual, and in such cases the individual is assigned a haplotype pair containing this known haplotype and a new haplotype derived by subtracting the known haplotype from the possible haplotype pair. Alternatively, the haplotype pair in an individual may be predicted from the individual's genotype for that gene using reported methods (e.g., Clark et al. 1990, Mol Bio Evol 7:111-22 or WO 01/80156) or through a commercial haplotyping service such as offered by Genaissance Pharmaceuticals, Inc. (New Haven, Conn.). In rare cases, either no haplotypes in the reference-population are consistent with the possible haplotype pairs, or alternatively, multiple reference haplotype pairs are consistent with the possible haplotype pairs. In such cases, the individual is preferably haplotyped using a direct molecular haplotyping method such as, for example, CLASPER System™ technology (U.S. Pat. No. 5,866,404), SMD, or allele-specific long-range PCR (Michalotos-Beloin et al., supra).

Determination of the number of haplotypes present in the individual from the genotypes is illustrated here for a haplotype containing two polymorphic sites, PSA and PSB. The Table below shows the 9 (3ⁿ, where each of n=2 bi-allelic polymorphic sites may have one of 3 different genotypes present) genotypes that may be detected at PSA and PSB, using both chromosomal copies from an individual. Eight of the nine possible genotypes for the two sites allow unambiguous determination of the number of copies of the haplotype present in the individual and therefore would allow unambiguous determination of whether the individual has a haplotype marker X. However, an individual with the C/G A/C genotype could possess either of the following haplotype pairs: CA/GC or CC/GA, and thus could have either 1 copy of the haplotype (CC/GA haplotype pair) corresponding to a haplotype marker X, or 0 copy (CA/GC haplotype pair) of the haplotype corresponding to a haplotype marker X. For this instance where there is ambiguity in the haplotype pair underlying the determined genotype C/G A/C, frequency information may be used to determine the most probable haplotype pair and therefore the most likely number of copies of the haplotype in the individual. If a particular haplotype pair consistent with the genotype of the individual is more frequent in a reference population than others consistent with the genotype, then that haplotype pair with the highest frequency is the most likely to be present in the individual. The copy number of the haplotype of interest in this haplotype pair can then be determined by visual inspection of the alleles at the PS that comprise the response marker for each haplotype in the pair.

Alternatively, for the ambiguous double heterozygote, genotyping of one or more additional sites in the gene or chromosomal locus may be performed to eliminate the ambiguity in deconvoluting the haplotype pairs underlying the genotype at PSA and PSB. The skilled artisan would recognize that these one or more additional sites would need to have sufficient linkage with the alleles in at least one of the possible haplotypes in the pair to permit unambiguous assignment of the haplotype pair. Although this illustration has been directed to the particular instance of determining the number of this haplotype present in an individual, the process would be analogous for any linked or substitute haplotypes comprising a haplotype marker X.

Possible copy numbers of a hypothetical Haplotype
(GA) based on the genotypes at PSA and PSB

| | | Copy Number of |
|---|---|---|
| PS4 | PS6 | Haplotype GA |
| G/G | C/C | 0 |
| G/G | A/C | 1 |
| C/G | C/C | 0 |
| C/G | A/C | 1 or 0 |
| G/C | A/A | 1 |
| G/G | A/A | 2 |
| C/C | A/A | 0 |
| C/C | A/C | 0 |
| C/C | C/C | 0 |

The individual's genotype for the desired set of PS may be determined using a variety of methods well-known in the art. Such methods typically include isolating from the individual a genomic DNA sample comprising both copies of the gene or locus of interest, amplifying from the sample one or more target regions containing the polymorphic sites to be genotyped, and detecting the nucleotide pair present at each PS of interest in the amplified target region(s). It is not necessary to use the same procedure to determine the genotype for each PS of interest.

In addition, the identity of the allele(s) present at any of the polymorphic sites described herein may be indirectly determined by haplotyping or genotyping another polymorphic site that is in linkage disequilibrium with the polymorphic site that is of interest. Polymorphic sites in linkage disequilibrium with the presently disclosed polymorphic sites may be located in regions of the gene or in other genomic regions not examined herein. Detection of the allele(s) present at a polymorphic site in linkage disequilibrium with the novel polymorphic sites described herein may be performed by, but is not limited to, any of the above-mentioned methods for detecting the identity of the allele at a polymorphic site.

Alternatively, the presence in an individual of a haplotype or haplotype pair for a set of PS comprising a haplotype marker X may be determined by directly haplotyping at least one of the copies of the individual's genomic region of interest, or suitable fragment thereof, using methods known in the art. Such direct haplotyping methods typically involve treating a genomic nucleic acid sample isolated from the individual in a manner that produces a hemizygous DNA sample that only has one of the two “copies” of the individual's genomic region which, as readily understood by the skilled artisan, may be the same allele or different alleles, amplifying from the sample one or more target regions containing the polymorphic sites to be genotyped, and detecting the nucleotide present at each PS of interest in the amplified target region(s). The nucleic acid sample may be obtained using a variety of methods known in the art for preparing hemizygous DNA samples, which include: targeted in vivo cloning (TIVC) in yeast as described in WO 98/01573, U.S. Pat. No. 5,866,404, and U.S. Pat. No. 5,972,614; generating hemizygous DNA targets using an allele specific oligonucleotide in combination with primer extension and exonuclease degradation as described in U.S. Pat. No. 5,972,614; single molecule dilution (SND) as described in Ruaño et al., Proc. Natl. Acad. Sci. 87:6296-6300, 1990; and allele specific PCR (Ruaño et al., 1989, supra; Ruaño et al., 1991, supra; Michalatos-Beloin et al., supra).

As will be readily appreciated by those skilled in the art, any individual clone will typically only provide haplotype information on one of the two genomic copies present in an individual. If haplotype information is desired for the individual's other copy, additional clones will usually need to be examined. Typically, at least five clones should be examined to have more than a 90% probability of haplotyping both copies of the genomic locus in an individual. In some cases, however, once the haplotype for one genomic allele is directly determined, the haplotype for the other allele may be inferred if the individual has a known genotype for the polymorphic sites of interest or if the haplotype frequency or haplotype pair frequency for the individual's population group is known.

While in direct haplotyping of both copies of the gene, the analysis is preferably performed with each copy of the gene being placed in separate containers, it is also envisioned that if the two copies are labeled with different tags, or are otherwise separately distinguishable or identifiable, it could be possible in some cases to perform the haplotyping in the same container. For example, if first and second copies of the gene are labeled with different first and second fluorescent dyes, respectively, and an allele-specific oligonucleotide labeled with yet a third different fluorescent dye is used to assay the polymorphic site(s), then detecting a combination of the first and third dyes would identify the polymorphism in the first gene copy while detecting a combination of the second and third dyes would identify the polymorphism in the second gene copy.

The nucleic acid sample used in the above indirect and direct haplotyping methods is typically isolated from a biological sample taken from the individual, such as a blood sample or tissue sample. Suitable tissue samples include whole blood, saliva, tears, urine, skin and hair.

B. Pharmacogenomics

Knowledge of the particular alleles associated with a response to a particular treatment regime, alone or in conjunction with information on other genetic defects contributing to the particular disease or condition, allows for a customization of the prevention or treatment regime in accordance with the subject's genetic profile. The present invention relates, in particular, to the field of pharmacogenomics, i.e., to the study of how a patient's genes determine his or her response to a drug (e.g., a patient's “drug response phenotype”, or “drug response genotype”). Thus, another aspect of the invention provides methods for tailoring a subject's prophylactic or therapeutic treatment with a treatment regime (such as administration of Alefacept) according to the subject's drug response genotype. The pharmacogenomic methods of the invention allow a clinician or physician to target prophylactic or therapeutic treatments to patients who will most benefit from the treatment and to avoid treatment of patients who will be unresponsive to the treatment and/or may experience toxic drug-related side effects.

Based on the detection of one or more of the polymorphisms described herein in a sample derived from a subject, the response of the subject to a treatment regime may be predicted. For example, as indicated above, the presence of: (a) at least one copy of haplotype marker 1; (b) the presence of two copies of haplotype marker 2; (c) the presence of at least one copy of haplotype marker 4; or (d) the absence of haplotype marker 3 in a sample derived from a subject would indicate that the subject is likely to be unresponsive to treatment with Alefacept. Thus, this subject would be treated with another therapeutic regimen. In contrast, detection in a sample derived from a subject of: (a) the absence of haplotype marker 1; (b) the absence of haplotype marker 2 or the presence of one copy of haplotype marker 2; (c) the presence of at least one copy of haplotype marker 3; or (d) the absence of haplotype marker 4, would indicate that the subject is likely to be responsive to treatment with Alefacept. Thus, a physician treating this subject would elect to proceed with the treatment.

Predictive methods which employ the detection of a combination of any of the polymorphisms or haplotypes identified herein are also encompassed by the present invention. For example, the invention provides a method for identifying a subject who is likely to be unresponsive to treatment with Alefacept by determining: (a) the presence of haplotype marker 1 and haplotype marker 2 or (b) the presence of haplotype marker 1 and haplotype marker 3 in a sample derived from the subject. The invention also provides a method for identifying a subject who is likely to be responsive to treatment with Alefacept by determining: (a) the absence of haplotype marker 1 and haplotype marker 2 or (b) the absence of haplotype marker 1 and haplotype marker 3 in a sample derived from the subject.

In addition, the haplotype marker associations of the invention may be used to develop clinical trials for new treatments for skin diseases, e.g., psoriasis, and other disorders or diseases by allowing stratification of the patient population.

C. Kits

The invention also encompasses kits for detecting the presence of the haplotype markers of the invention in a biological sample, e.g., kits suitable for diagnosing the response of a subject to a treatment regime. The kits include a means for detecting the presence or absence of the haplotype marker of the invention in a sample obtained from a patient. Optionally, the kit may further include a data set of associations of the haplotype marker with the disease, disease susceptibility, or therapy response. In preferred embodiments, the data set of associations is on a computer-readable medium.

The invention further provides kits comprising at least one nucleic acid of the invention, preferably an oligonucleotide, more preferably an oligonucleotide primer or probe that may be used to detect a polymorphism or haplotype marker of the invention. In one embodiment, the kit may contain one or more oligonucleotides, including 5′ and 3′ oligonucleotides that hybridize 5′ and 3′ to at least one allele. PCR amplification oligonucleotides should hybridize between 25 and 2500 base pairs apart, preferably between about 100 and about 500 bases apart, in order to produce a PCR product of convenient size for subsequent analysis.

Often, the kits contain one or more pairs of oligonucleotide primers that hybridize to a target nucleic acid to allow amplification of one or more regions of the target that contain or are a portion of one or more haplotype markers of the invention. In preferred embodiments, the amplification product could be analyzed to determine the genotype of the polymorphisms and/or haplotype marker contained within the target nucleic acid. In some kits, oligonucleotide probes are provided immobilized on a substrate. In preferred embodiments, an oligonucleotide probe immobilized on a substrate hybridizes to a specific allele of a given polymorphism of the invention.

For use in a kit, oligonucleotides may be any of a variety of natural and/or synthetic compositions such as synthetic oligonucleotides, restriction fragments, cDNAs, synthetic peptide nucleic acids (PNAs), and the like. The assay kit and method may also employ labeled oligonucleotides to allow ease of identification in the assays. Examples of labels which may be employed include radio-labels, enzymes, fluorescent compounds, streptavidin, avidin, biotin, magnetic moieties, metal binding moieties, antigen or antibody moieties, and the like.

The kit may, optionally, also include DNA sampling means. DNA sampling means are well known to one of skill in the art and can include, but not be limited to, substrates such as filter papers, (e.g., the AmpliCard™ (University of Sheffield, Sheffield, England S10 2JF; Tarlow, J W, et al., J. of Invest. Dermatol. 103:387-389 (1994)) and the like; DNA purification reagents such as the Nucleon™ kits, lysis buffers, proteinase solutions and the like; PCR reagents, such as 10× reaction buffers, thermostable polymerase, dNTPs, and the like; and allele detection means such as the HinfI restriction enzyme, allele specific oligonucleotides, degenerate oligonucleotide primers for nested PCR from dried blood. Usually, the kit also contains instructions for carrying out the methods of the invention. These kits facilitate the identification of subjects that are likely to respond positively or negatively to a treatment regime; those at risk of developing an inflammatory disease, such as psoriasis, those sensitive to drugs that exacerbate psoriatic symptoms, and those with other phenotypic traits in linkage disequilibrium with the polymorphisms and haplotype markers of the invention, and could also be useful for genetic counseling.

The contents of all references, patents and published patent applications cited throughout this application, as well as the Figures, are incorporated herein by reference.

This invention is further illustrated by the following examples, which should not be construed as limiting.

### EXAMPLES

The following methods were used in the Examples described herein.

A. Study Subjects

In the first study, patients (N=205) with sufficient DNA were selected from 4 studies, 3 of which were placebo-controlled studies and 1 of which was an open label study. Of the total, 145 patients had been treated with Alefacept and 60 patients had been treated with placebo. Of the 145 active patients, there were 119 strong responders and 26 non-responders. Of the 60 placebo patients, there were 30 strong responders and 30 non-responders.

In the second study, the clinical cohort was composed of 68 strong responders, who were randomly selected from a set of active patients who achieved PASI75 in response to Alefacept, and 26 non-responders from the first study.

B. Sample Acquisition and Processing

Based on the definition of the clinical phenotype described here, only strong responders and non-responders were included in the analysis population. Subjects with low DNA quantities were excluded from the analysis set. To minimize undesired noise in the clinical phenotype definition, non-responders who did not achieve PASI25 at any time during the treatment course due to limited efficacy visits and/or usage of prohibited concomitant medications were excluded from the final analysis set.

C. Phenotypes Analyzed

The percent reduction in the Psoriasis Area and Severity Index (PASI) from baseline was the phenotype that was evaluated in the first and second studies. The response categories were defined as follows: (a) a strong responder refers to a patient with a response of greater than or equal to 50% reduction of PASI from baseline at any time; (b) a partial responder refers to a patient with a response of greater than or equal to 25% but <50% reduction of PASI from baseline at any time; or (c) a non-responder refers to a patient with a response of less than 25% reduction of PASI from baseline at any time.

D. Candidate Genes

In the first study, a set of candidate genes for genotyping was selected, focusing on genes involved in T cell activation and inhibition (T-cell receptor, co-receptors, integrins), receptors targeted by the drug (Fc gamma receptors), and genes known to be linked to psoriasis. In the second study, a set of additional 39 candidate genes for genotyping was selected.

Candidate genes for genotyping analysis focused on genes that are linked to the intended disease for treatment, psoriasis, and on genes that encode proteins that interact directly with Amevive and those that are activated secondary to Amevive binding to its target receptors. Genes linked to psoriasis included the PSORS1 locus genes on chromosome 6, namely, HCR, SPR1, STG, SEEK 1, TCF 19 and HLA-C. Genes selected based on Amevive's mode of action included CD2, the cognate receptor for Amevive and CD58, the LFA3 gene that encodes the membrane bound form of Amevive. In addition, genes encoding cell surface receptor proteins involved in T lymphocyte activation and co-stimulation, such as CD3E, CD3G, CD3Z, CD4, CD8A, CD8B1, CTLA4, CCR6, ICAM and ICOS, and those encoding down stream signal proteins, such as NFKB1, NFKB2, LCK, TNF, IL-20, CD2BP, IKBKAP, ZAP70, ITGAL, ITGAM, were studied. Based on the ability of the C-terminus of the Amevive molecule to engage Fc gamma receptors to mediate effector functions (one of the proposed clinical modes of action), genes encoding Fc gamma receptors I (A,B), II (A,B) and III (A,B) as well as genes encoding proteins that mediate down stream signals and Fc receptor mediated effector functions, such as MAPK1, NFATC1, NFATC2, GNLY (granulolysin), GZMB (granzyme-B), were studied.

E. SNP Discovery and Haplotype Marker Generation

SNP discovery and genotyping of the clinical cohort was carried out by sequencing genomic DNA from subjects in the cohort. Regions targeted for sequencing (500 bp upstream of the ATG; each exon plus 100 bp of flanking sequence on each end of the exon; and 100 bp downstream of the termination codon) were amplified from genomic DNA. Tailed PCR primers were designed using the sequence of each of the candidate genes. Amplified PCR products were sequenced using Applied Biosystems Big Dye Terminator chemistry and analyzed on an ABI Prism 3700 DNA Analyzer. Sequences obtained were examined for the presence of polymorphisms using the PolyPhred program. Subsequently, the sequence data was scanned manually for sample preparation and sequencing anomalies and SNPs incorrectly identified in the PolyPhred output file were discarded. Once an SNP was accepted, the genotype of each individual in the clinical cohort was manually verified and stored.

Haplotypes were derived from the SNP genotypes of the clinical cohort using the method described in WO 01/80156. For each assignment of a pair of haplotypes to a subject, a confidence score quantified the likelihood of accuracy of the assignment. SNPs with low frequency were not used in building haplotypes.

F. Statistical Analysis

For each gene, a reduced set of polymorphic sites yielding at least 95% of the genetic diversity of the haplotypes derived above for the locus using all polymorphic sites (Judson, R. et al. Pharmacogenomics, 3(3):379-91 (2002)) was selected for statistical analysis with the clinical endpoint. All possible haplotypes of each gene containing up to a maximum of four polymorphisms from the reduced set were enumerated. This upper limit on the number of polymorphisms was used because for a given gene, haplotypes involving more than 4 or 5 polymorphic sites are rarely the most powerful haplotypes, and because less important polymorphisms are often added to already powerful haplotypes, diluting their effects. Each individual in the analysis cohort was classified as having 0, 1, or 2 copies of the haplotype. Each unique haplotype with a frequency of >5% was then tested for association with the clinical endpoint.

One primary outcome variable was a dichotomized version of the PASI score (See Section C. Phenotypes Analyzed), including non-responders and strong responders. Patients whose response fell between these two ranges were not included in the analysis.

Logistic regression was used to assess associations between haplotypes and the binary outcome of strong response or non-response. Models used in the logistic regression to analyze association between haplotypes and clinical phenotypes were a general association model (0 copy vs. 1 copy vs. 2 copies ), a dominant association model (0 copy vs. 1 or 2 copies), and a recessive association model (0 or 1 copy vs. 2 copies). For all models, a genetic marker term for haplotype copy number was included. Gender and baseline PASI were used as covariates. Since many statistical tests were performed, permutation tests (Good, P, 2000. Permutation Tests: A Practical Guide to Resampling Methods for Testing Hypotheses, 2nd edition. Springer Series in Statistics, New York) were performed to adjust for multiple comparisons, while appropriately accounting for the non-independence of the haplotypes in that gene. In this procedure, the outcome and covariates were held constant, and the set of haplotypes generated was randomly permuted 1,000 times. The minimum p-value from among the many haplotypes was noted for each of the 1,000 permutations. Then an observed p-value's quantile in this distribution was used as the adjusted p-value. For example, if 4.5% of the minimum p-values from the permutations were smaller than a haplotype's raw p-value, then that haplotype's permutation adjusted p-value would be 0.045.

Haplotypes found to have associations with a clinical phenotype are labeled herein based on the location of the contributing SNPs in each gene using the initiation codon (ATG) for the reference mRNA for that gene as the reference for the +1 position. The notation used gives the ATG offset for the SNPs (5′ to 3′) followed by the allele at each position. For example, in the haplotype marker 1 (−255,25,28589/CTG) in the CD8B1 gene, C is the allele at a promoter SNP at −255, and T and G are the alleles at SNPs at positions 25 and 28589, respectively, in the gene.

The following Examples provide Tables containing a summary of the polymorphic sites identified in the CD8B1, HCR, SPR1, and TCF19 genes. In particular, for each gene, a Table is provided with the polymorphic site number (“Polymorphic Site Number”), the ATG offset of the first position of the SNP (“ATG Offset”), the nucleotide position of the first-position of the SNP within the sequence (“Nucleotide Position”), the allele present at the ATG offset and nucleotide position (“Reference Allele”), and the allele that is substituted in place of the reference allele (“Variant Allele”).

In the present analyses, the CD8B1, HCR and SPR1 and the TCF19 genes were discovered to exhibit statistically significant association to the PASI scores.

### Example I

### Identification and Analysis of Haplotype Marker 1 in the CD8B1 Gene

This Example describes the analysis of haplotypes in the CD8B1 gene for association with respect to response to Alefacept and the identification that the copy number of these haplotypes can differentiate strong responders to Alefacept from non-responders. Haplotype marker 1 of Table 1, a three-SNP haplotype, is analyzed in greatest detail.

A. Polymorphic Sites Identified in the CD8B1 Gene

Table 2 depicts the polymorphic sites identified in the CD8B1 gene. As set forth above, Table 2 provides a polymorphic site number (“Polymorphic Site Number”), the ATG offset of the first position of the SNP (“ATG Offset”), the nucleotide position of the first position of the SNP within SEQ ID NO:1 (“Nucleotide Position”), the allele present at the ATG offset and nucleotide position (“Reference Allele”), and the allele that is substituted in place of the reference allele (“Variant Allele”).

TABLE 2
Polymorphic Sites Identified in the CD8B1 Gene

| Polymorphic | ATG | Nucleotide | Reference | Variant |
|---|---|---|---|---|
| Site Number | Offset | Position | Allele | Allele |
| PS2 | −685 | 1272 | A | G |
| PS6 | −255 | 1702 | C | T |
| PS11 | 25 | 1981 | C | T |
| PS13 | 8632 | 9682 | G | A |
| PS15 | 15080 | 12027 | G | A |
| PS21 | 19501 | 16448 | G | A |
| PS26 | 28589 | 25065 | A | G |
| PS27 | 28663 | 25139 | C | T |
| PS28 | 28739 | 25215 | C | T |

Tables 3A and 3B provide the CD8B1 haplotypes that showed the most significant associations to PASI using a dominant and recessive genetic copy number model, respectively. In particular, the “Polymorphic Sites of CD8B1 Gene” set forth in the columns of Tables 3A and 3B correspond with the polymorphic sites of the CD8B1 gene that are identified in Table 2. Each row of Tables 3A and 3B represents a haplotype marker. In addition, “Unadjusted P-Value” and “O.R.” correspond to the raw p-value and odds ratio, respectively, for each haplotype marker within the CD8B1 gene. The asterisks in Tables 3A and 3B denote that the alleles at these sites are not determining and may be either allele, i.e., either the Reference Allele or the Variant Allele, as identified in Table 2. The “Lower CI of O.R.” and “Upper CI of O.R.” represent 95% confidence limits of the odds ratio.

TABLE 3A
Table of CD8B1 haplotypes showing association to PASI using a dominant genetic copy number model

| PS2 | PS6 | PS11 | PS13 | PS15 | PS21 | PS26 | PS27 | PS28 | Unadjusted | O.R. | Lower CI | Upper CI |
|---|---|---|---|---|---|---|---|---|---|---|---|---|
| −685 | −255 | 25 | 8632 | 15080 | 19501 | 28589 | 28663 | 28739 | P-Value | (1 or 2 vs 0) | of O.R. | of O.R. |
| * | C | T | G | * | * | G | * | * | 0.0004 | 0.17 | 0.06 | 0.45 |
| * | C | T | * | * | A | G | * | * | 0.0006 | 0.18 | 0.07 | 0.47 |
| * | * | T | G | * | A | G | * | * | 0.0006 | 0.18 | 0.07 | 0.48 |
| * | C | T | * | * | * | G | * | * | 0.0009 | 0.19 | 0.07 | 0.51 |
| * | * | T | G | * | * | G | * | * | 0.0010 | 0.20 | 0.08 | 0.52 |
| * | * | T | * | * | A | G | * | * | 0.0014 | 0.21 | 0.08 | 0.55 |
| * | C | * | G | * | A | G | * | * | 0.0020 | 0.21 | 0.08 | 0.57 |
| A | * | * | G | A | * | G | * | * | 0.0020 | 0.22 | 0.08 | 0.57 |
| * | * | T | * | * | * | G | * | * | 0.0022 | 0.23 | 0.09 | 0.59 |
| * | * | T | G | A | * | G | * | * | 0.0023 | 0.22 | 0.09 | 0.58 |
| * | C | * | G | * | * | G | C | * | 0.0026 | 0.22 | 0.09 | 0.59 |
| A | C | * | G | * | * | G | * | * | 0.0028 | 0.23 | 0.09 | 0.61 |
| * | * | * | G | A | * | G | C | * | 0.0028 | 0.23 | 0.09 | 0.60 |
| * | C | * | G | A | * | G | * | * | 0.0028 | 0.23 | 0.09 | 0.60 |
| A | * | * | G | A | * | * | C | * | 0.0029 | 0.23 | 0.09 | 0.61 |
| * | C | * | G | * | * | G | * | * | 0.0030 | 0.23 | 0.09 | 0.61 |
| * | * | T | G | A | * | * | C | * | 0.0032 | 0.23 | 0.09 | 0.61 |
| * | * | * | G | A | * | G | * | * | 0.0033 | 0.24 | 0.09 | 0.62 |
| * | C | * | * | * | A | G | C | * | 0.0036 | 0.24 | 0.09 | 0.63 |
| * | * | * | G | * | A | G | C | * | 0.0039 | 0.25 | 0.10 | 0.64 |
| A | C | * | * | A | * | G | * | * | 0.0040 | 0.25 | 0.10 | 0.64 |
| * | C | * | * | * | A | G | * | * | 0.0042 | 0.24 | 0.09 | 0.64 |
| A | * | * | G | * | A | G | * | * | 0.0043 | 0.26 | 0.10 | 0.65 |
| * | C | T | * | A | * | G | * | * | 0.0045 | 0.25 | 0.10 | 0.65 |
| * | * | * | G | * | A | G | * | * | 0.0045 | 0.25 | 0.10 | 0.65 |
| A | * | * | * | A | * | G | * | * | 0.0047 | 0.26 | 0.10 | 0.66 |
| * | C | * | G | A | * | * | C | * | 0.0050 | 0.25 | 0.10 | 0.66 |
| * | C | T | * | * | * | * | C | C | 0.0043 | 0.25 | 0.10 | 0.65 |
| * | * | T | * | * | * | * | C | C | 0.0053 | 0.26 | 0.10 | 0.67 |

B. Haplotype Marker 1 in the CD8B1 Gene

A three-SNP haplotype (−255,25,28589/CTG), referred to herein as “haplotype marker 1,” in the CD8B1 gene differentiates strong responders to Alefacept from non-responders with an OR of 5.2.

The association between haplotype marker 1 with Alefacept response has a dominant genetic pattern in that subjects with 1 or 2 copies are more likely to be non-responders and subjects with 0 copy are more likely to be strong responders. A summary of the haplotype marker 1 association is provided in Table 4.

TABLE 4
Summary of Association Results of Haplotype Marker 1

| | Haplotype Marker 1 |
|---|---|
| | (−255, 25, 28589/CTG) |
| Subject Count (# Copy) | 96(0) 49(1, 2) |
| OR (0 vs. 1, 2) | 5.2 |
| 95% Confidence Interval | 1.97, 13.8 |
| Unadjusted p-value | 0.0009 |
| Permutation adjusted p-value | 0.021 |
| Copy number of haplotype marker | 1 or 2 copies |
| associated with non-response | |
| Frequency of 0 copies of the haplotype | 66.2% |
| marker in Alefacept Treated Cohort | |
| Percent of non-responders with 1 or 2 copies | 61.5% |
| Percent of strong responders with 1 or 2 copies | 27.7% |

C. Association Analysis of Haplotype Marker 1 with Drug Response

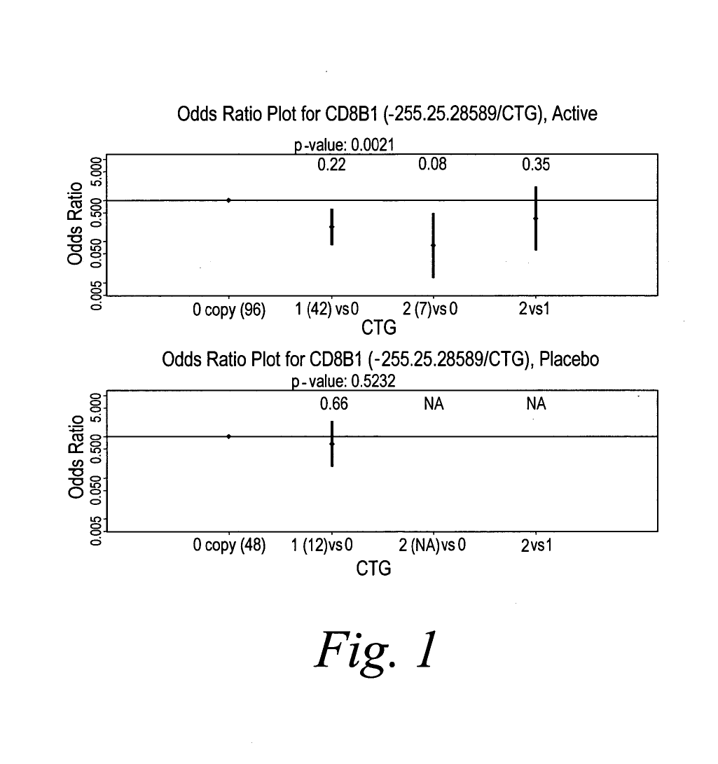

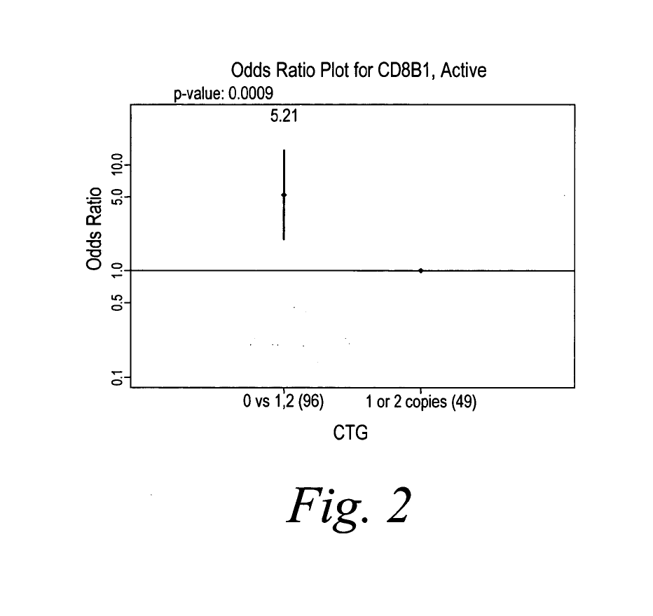

FIG. 1 depicts the results of the association analysis of haplotype marker 1 with drug response as an OR plot in a general model (comparing 0, 1, and 2 copies of the marker). FIG. 2 shows an OR plot of the association of the haplotype marker 1 using a dominant model of inheritance. This association has a raw p-value of 0.0021 when 0, 1 or 2 copies of the haplotype (general model) were tested with respect to response in the Alefacept treated patient group. The copy number of the haplotype marker had no effect on placebo response and the p-value for response to placebo is non-significant (p=0.523) (lower panel, FIG. 1). The association remained statistically significant (permutation adjusted p=0.021) after correcting the raw p value for multiple comparisons. When analyzed in the dominant model, people with zero copies of the marker were 5.2 times more likely to respond to Alefacept compared to subjects with either 1 or 2 copies of the marker. Gender imbalance existed in the patient cohort, therefore a subset analysis for males, the larger gender group, was performed and marker main effect remained significant (p=0.0044), with an OR of 6.1 for subjects with zero copies versus 1 or 2 copies of the haplotype marker.

The marker distribution between strong and non-responders to Alefacept is summarized in Table 5. The CD8B1 marker (1 or 2 copies of CTG) identifies 62% of the non-responders and 28% of the strong responders with an OR of 5.2. By applying these test characteristics to the distribution of response trials (23% non-responders and 55% strong responders), CD8B1 haplotype marker 1 is predicted to have a positive predictive value (PPV) (i.e., the probability of responding strongly, given that one has the strong response copy number) of 81.6% and a negative predictive value (NPV) (i.e., the probability of not responding, given that one has the non-response copy number) of 48.3% for non-response to Alefacept.

TABLE 5
Distribution of Haplotype Marker 1

| Copy Number | Non-Responder | Strong Responder |
|---|---|---|
| 0 | 10 | 86 |
| 1 or 2 | 16 | 33 |
| Total no. of Subjects | 26 | 119 |

### Example II

### Identification and Analysis of the Haplotype Marker 2 and Haplotype Marker 5 in the HCR Gene

This Example describes the identification and analysis of haplotypes in HCR which are associated with response to treatment with Alefacept. Haplotype markers 2 and 5, three-SNP haplotypes, were analyzed in most detail.

A. Polymorphic Sites Identified in the HCR Gene

Table 6 depicts the polymorphic sites identified in the HCR gene. As set forth above, Table 6 provides a polymorphic site number, the ATG offset of the first position of the SNP, the nucleotide position of the first position of the SNP within SEQ ID NO:2, the allele present at the ATG offset and nucleotide position, and the allele that is substituted in place of the reference allele.

TABLE 6
Polymorphic Sites Identified in the HCR Gene

| Polymorphic | ATG | Nucleotide | Reference | Variant |
|---|---|---|---|---|
| Site Number | Offset | Position | Allele | Allele |
| PS5 | 2173 | 2195 | G | A |
| PS6 | 2175 | 2197 | C | T |
| PS10 | 2360 | 2382 | G | C |
| PS13 | 5782 | 5804 | G | A |
| PS14 | 5787 | 5809 | T | A |
| PS16 | 6174 | 6196 | C | A |
| PS17 | 6666 | 6688 | A | G |
| PS18 | 8277 | 8299 | C | T |
| PS19 | 8440 | 8462 | C | T |
| PS22 | 8476 | 8498 | C | T |
| PS32 | 11565 | 11587 | T | G |
| PS38 | 11941 | 11963 | G | T |
| PS40 | 12152 | 12174 | G | A |
| PS43 | 13553 | 13575 | C | T |
| PS47 | 13892 | 13914 | A | T |
| PS50 | 14287 | 14309 | C | G |

Tables 7A and 7B provide the HCR haplotypes that showed the most significant associations to PASI using a dominant and recessive genetic copy number model, respectively. As described above, the “Polymorphic Sites of the HCR Gene” set forth in the columns of Tables 7A and 7B correspond with the polymorphic sites of the HCR gene that are identified in Table 6. Each row of Tables 7A and 7B represents a haplotype marker. In addition, the “Unadjusted P-Value” and “O.R.” correspond to the raw p-value and odds ratio data, respectively, for each haplotype marker within the HCR gene. The asterisks in Tables 7A and 7B denote that the alleles at these sites are not determining and may be either allele, i.e., either the Reference Allele or the Variant Allele, as identified in Table 6. The “Lower CI of O.R.” and “Upper CI of O.R.” represent 95% confidence limits of the odds ratio.

TABLE 7A
Table of HCR haplotypes showing association to
PASI using a dominant genetic copy number model

| PS5 | PS6 | PS10 | PS13 | PS14 | PS16 | PS17 | PS18 | PS19 | PS22 | PS32 | PS38 |
|---|---|---|---|---|---|---|---|---|---|---|---|
| 2173 | 2175 | 2360 | 5782 | 5787 | 6174 | 6666 | 8277 | 8440 | 8476 | 11565 | 11941 |
| * | * | * | * | * | A | * | * | T | * | * | * |
| * | * | * | * | * | * | * | * | * | * | * | * |
| * | * | * | * | * | * | * | * | * | * | * | * |

| PS40 | PS43 | PS47 | PS50 | Unadjusted | O.R. | Lower CI | Upper CI |
|---|---|---|---|---|---|---|---|
| 12152 | 13553 | 13892 | 14287 | P-Value | (1 or 2 vs 0) | of O.R. | of O.R. |
| * | * | A | * | 0.0031 | 4.3 | 1.6 | 11.2 |
| * | * | * | G | 0.0037 | 4.0 | 1.6 | 10.3 |
| G | * | * | G | 0.0069 | 3.6 | 1.4 | 9.0 |

TABLE 7B
Table of HCR haplotypes showing association to PASI
using a recessive genetic copy number model

| PS5 | PS6 | PS10 | PS13 | PS14 | PS16 | PS17 | PS18 | PS19 | PS22 | PS32 | PS38 |
|---|---|---|---|---|---|---|---|---|---|---|---|
| 2173 | 2175 | 2360 | 5782 | 5787 | 6174 | 6666 | 8277 | 8440 | 8476 | 11565 | 11941 |
| * | * | * | * | * | * | * | * | * | * | G | * |
| * | C | * | * | T | * | * | * | * | * | G | * |
| * | * | * | G | * | * | * | * | * | * | G | * |
| * | C | * | G | * | * | * | * | * | * | G | * |
| * | C | * | * | * | * | * | * | * | * | G | * |
| * | * | * | * | * | * | * | * | * | * | G | * |
| G | * | * | * | * | * | * | * | * | * | G | * |
| G | C | * | * | * | * | * | * | * | * | G | * |
| * | C | * | * | * | * | * | * | * | * | G | * |
| * | * | * | G | * | * | * | * | * | * | * | * |
| * | * | * | * | * | * | A | * | * | * | G | * |
| * | C | * | * | T | A | * | * | * | * | * | * |
| * | * | * | G | * | * | A | * | * | * | G | * |
| * | C | * | G | T | * | * | * | * | * | * | * |
| * | C | * | * | T | * | * | * | * | * | * | * |
| * | * | * | * | * | * | * | * | * | * | * | * |
| * | * | * | * | * | A | * | * | * | * | * | * |
| * | C | * | * | * | * | * | * | * | * | G | * |
| * | * | * | * | * | * | * | * | * | C | G | * |
| * | * | * | * | * | * | * | * | * | * | G | G |
| G | * | * | * | * | * | * | * | * | * | * | * |
| * | C | * | * | T | * | * | * | * | * | * | * |
| * | * | * | G | * | * | * | * | * | C | G | * |
| * | * | * | G | * | * | * | * | * | * | * | * |
| * | * | * | G | * | * | * | * | * | * | G | G |
| G | * | * | * | * | * | A | * | * | * | G | * |
| G | * | * | G | * | * | * | * | * | * | * | * |
| * | C | * | * | * | A | * | * | * | * | * | * |
| * | * | * | * | * | * | A | * | * | * | G | * |
| G | * | * | * | * | * | * | * | * | C | G | * |
| * | * | * | * | * | * | * | C | * | C | G | * |
| * | * | * | * | * | * | * | * | * | * | * | * |
| G | * | * | * | * | * | * | * | * | * | G | G |
| * | C | * | * | T | * | * | * | * | * | * | * |
| * | * | * | * | * | * | * | * | * | * | G | G |
| * | * | * | * | * | * | * | * | * | C | G | * |
| * | C | * | G | * | * | * | * | * | * | * | * |
| G | * | * | * | * | * | * | * | * | * | * | * |
| G | * | c | * | T | * | * | * | * | * | * | * |
| * | * | c | * | T | * | * | * | * | * | * | * |
| G | C | * | * | * | A | * | * | * | * | * | * |
| G | C | * | * | T | * | * | * | * | * | * | * |
| * | C | * | * | * | A | * | * | * | * | * | * |

| PS40 | PS43 | PS47 | PS50 | Unadjusted | O.R. | Lower CI | Upper CI |
|---|---|---|---|---|---|---|---|
| 12152 | 13553 | 13892 | 14287 | P-Value | (2 vs 1 or 0) | of O.R. | of O.R. |
| * | C | * | C | 0.0003 | 0.17 | 0.06 | 0.44 |
| * | * | * | * | 0.0003 | 0.17 | 0.07 | 0.45 |
| * | * | * | C | 0.0004 | 0.17 | 0.07 | 0.46 |
| * | * | * | * | 0.0004 | 0.18 | 0.07 | 0.46 |
| * | C | * | * | 0.0005 | 0.18 | 0.07 | 0.47 |
| * | * | * | C | 0.0006 | 0.19 | 0.07 | 0.49 |
| * | * | * | C | 0.0006 | 0.18 | 0.07 | 0.49 |
| * | * | * | * | 0.0008 | 0.19 | 0.07 | 0.50 |
| * | * | * | * | 0.0009 | 0.20 | 0.08 | 0.51 |
| * | C | * | C | 0.0014 | 0.21 | 0.08 | 0.55 |
| * | C | * | * | 0.0017 | 0.22 | 0.08 | 0.56 |
| * | * | * | * | 0.0018 | 0.20 | 0.08 | 0.55 |
| * | * | * | * | 0.0020 | 0.23 | 0.09 | 0.58 |
| * | * | * | * | 0.0020 | 0.23 | 0.09 | 0.59 |
| G | * | * | * | 0.0021 | 0.23 | 0.09 | 0.59 |
| * | C | * | C | 0.0021 | 0.22 | 0.09 | 0.58 |
| * | C | * | C | 0.0022 | 0.20 | 0.07 | 0.56 |
| G | * | * | * | 0.0023 | 0.23 | 0.09 | 0.59 |
| * | C | * | * | 0.0023 | 0.22 | 0.09 | 0.59 |
| * | C | * | * | 0.0023 | 0.23 | 0.09 | 0.59 |
| * | C | * | C | 0.0023 | 0.22 | 0.08 | 0.58 |
| * | C | * | * | 0.0024 | 0.23 | 0.09 | 0.60 |
| * | * | * | * | 0.0024 | 0.23 | 0.09 | 0.59 |
| * | * | * | C | 0.0025 | 0.23 | 0.09 | 0.60 |
| * | * | * | * | 0.0027 | 0.24 | 0.09 | 0.61 |
| * | * | * | * | 0.0027 | 0.23 | 0.09 | 0.60 |
| * | * | * | C | 0.0028 | 0.23 | 0.09 | 0.61 |
| * | C | * | * | 0.0029 | 0.23 | 0.09 | 0.61 |
| * | * | * | * | 0.0030 | 0.24 | 0.09 | 0.62 |
| * | * | * | * | 0.0032 | 0.23 | 0.09 | 0.61 |
| * | * | * | * | 0.0036 | 0.25 | 0.10 | 0.63 |
| * | * | * | C | 0.0037 | 0.25 | 0.10 | 0.64 |
| * | * | * | * | 0.0037 | 0.24 | 0.09 | 0.63 |
| * | * | * | * | 0.0040 | 0.26 | 0.10 | 0.65 |
| * | * | * | * | 0.0040 | 0.25 | 0.10 | 0.64 |
| * | * | * | * | 0.0040 | 0.25 | 0.10 | 0.64 |
| * | C | * | * | 0.0041 | 0.25 | 0.10 | 0.65 |
| * | * | * | C | 0.0042 | 0.25 | 0.10 | 0.64 |
| * | * | * | * | 0.0045 | 0.22 | 0.08 | 0.62 |
| * | * | * | * | 0.0046 | 0.23 | 0.08 | 0.64 |
| * | * | * | * | 0.0046 | 0.24 | 0.09 | 0.65 |
| * | * | * | * | 0.0047 | 0.26 | 0.10 | 0.66 |
| * | * | * | * | 0.0053 | 0.26 | 0.10 | 0.67 |

B. Haplotype Marker 2 in the HCR Gene

A summary of the marker (2175,5787,11565/CTG), referred to herein as “haplotype marker 2,” is provided in Table 8. Haplotype marker 2 has a recessive pattern in that subjects with 2 copies are more likely to be non-responders. A second marker (5782,11565,14287/GGC), also referred to herein as “haplotype marker 5,” with similar distribution and effect on response as marker (2175,5787,11565/CTG) is also summarized in Table 9.

TABLE 8
Summary of the Association Results for Haplotype Marker 2

| | Haplotype Marker 2 |
|---|---|
| | (2175, 5787, 11565/CTG) |
| Subject Count (# Copy) | 105(0, 1) 40(2) |
| OR (0, 1 vs 2) | 5.7 |
| 95% Confidence Interval | 2.2, 15 |
| Unadjusted p-value | 0.0003 |
| Permutation adjusted p-value | 0.031 |
| Copy number of haplotype associated | 2 copies |
| with non-response | |
| Frequency of 0 or 1 copies of the | 72.4% |
| marker in Alefacept Treated Cohort | |
| Percent of non-responders with 2 copies | 57.7% |
| Percent of strong responders with 2 copies | 21.0% |

TABLE 9
Summary of the Association Results of Haplotype Marker 5

| | Haplotype Marker 5 |
|---|---|
| | 5782, 11565, 14287/GGC |
| Subject Count (# Copy) | 109(0, 1) 36(2) |
| OR (0, 1 vs 2) | 5.7 |
| 95% Confidence Interval | 2.2, 15.1 |
| Unadjusted p-value | 0.0004 |
| Permutation adjusted p-value | 0.033 |
| Copy number of haplotype marker | 2 copies |
| associated with non-response | |
| Frequency of 0 or 1 copies of the haplotype | 75.2% |
| marker in Alefacept Treated Cohort | |
| Percent of non-responders with 2 copies | 53.8% |
| Percent of strong responders with 2 copies | 18.5% |

C. Association Analysis of Haplotype Marker 2 with Drug Response

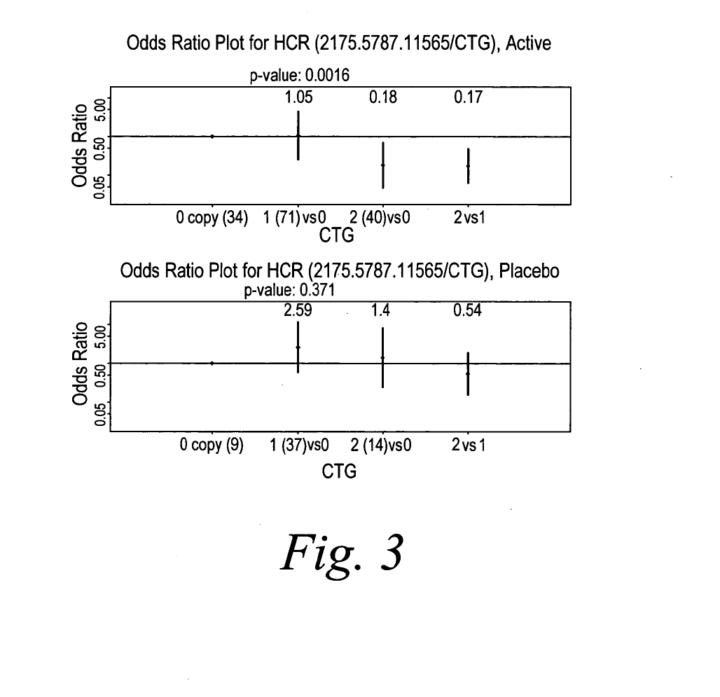

FIG. 3 depicts the results of the association analysis of haplotype marker 2, with drug response as an OR plot. The OR of response to Alefacept for the population of patients with 2 copies of the marker is 5.5 compared to those with 0 or 1 copies. The selected haplotype has a raw p-value of 0.0016 when 0, 1 or 2 copies of the haplotype marker were tested against response in the Alefacept treated patient group. The copy number of the haplotype marker has no effect on placebo response and the p-value for response to placebo is non-significant (p=0.371) (lower panel, FIG. 3). The association remained statistically significant (permutation adjusted p=0.031) after correcting the raw p value for multiple comparisons.

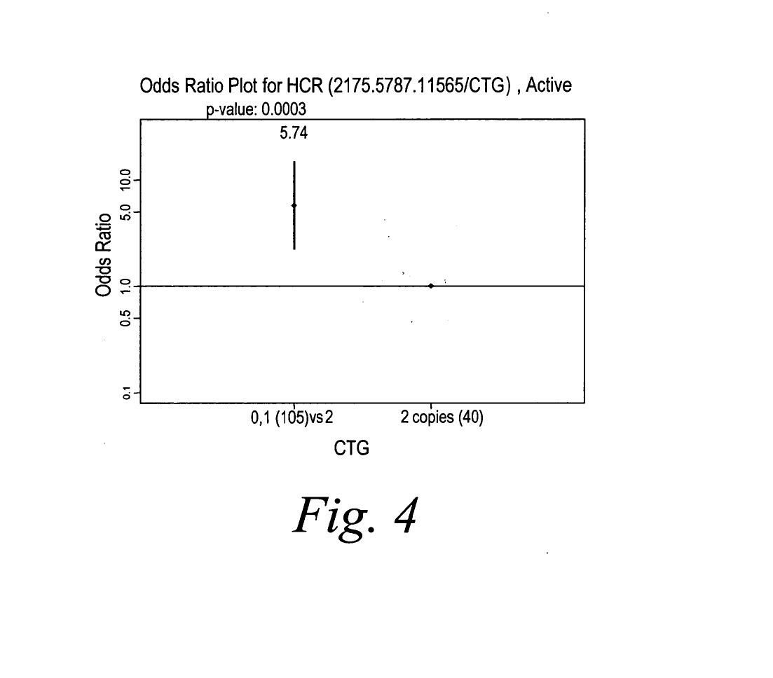

Subjects with 0 or 1 copies of the haplotype marker may be collapsed into 1 group due to a similar likelihood of response, as seen in FIG. 3. This implies a recessive model of inheritance where subjects with 0 or 1 copy of the haplotype marker are 5.7 times more likely to respond to Alefacept compared to those with 2 copies of the haplotype marker (FIG. 4).

The marker distribution between strong and non-responders to Alefacept is summarized in Table 10. This HCR marker (2 copies of CTG) identifies 58% of the non-responders and 21% of the strong responders with an OR of 5.7. If these test characteristics are applied to the distribution of response in the Alefacept trials (23% non-responders and 55% strong responders), HCR haplotype marker 2 would be predicted to have a PPV of 81.1% and NPV of 52% for non-response to Alefacept.

TABLE 10
Distribution of Haplotype Marker 2

| Copy Number | Non-Responder | Strong Responder |
|---|---|---|
| 0 or 1 | 11 | 94 |
| 2 | 15 | 25 |
| Total no. of Subjects | 26 | 119 |

### Example III

### Identification and Analysis of the Haplotype Marker 3 in the SPR1 Gene

This Example describes the identification and analysis of haplotypes in SPR1 which are strongly associated with response to treatment with Alefacept. Haplotype marker 3, a three-SNP haplotype, is analyzed in greatest detail.

A. Polymorphic Sites Identified in the SPR1 Gene

Table 11 depicts the polymorphic sites identified in the SPR1 gene. As set forth above, Table 11 provides a polymorphic site number, the ATG offset of the first position of the SNP, the nucleotide position of the first position of the SNP within SEQ ID NO:3, the allele present at the ATG offset and nucleotide position, and the allele that is substituted in place of the reference allele.

TABLE 11
Polymorphic Sites Identified in the SPR1 Gene

| Polymorphic | ATG | Nucleotide | Reference | Variant |
|---|---|---|---|---|
| Site Number | Offset | Position | Allele | Allele |
| PS2 | −1119 | 1128 | G | A |
| PS6 | −845 | 1402 | G | A |
| PS7 | −455 | 1792 | G | A |
| PS12 | −384 | 1863 | T | C |
| PS16 | −228 | 2019 | A | G |
| PS20 | 161 | 2407 | C | T |
| PS21 | 627 | 2873 | G | A |
| PS22 | 739 | 2985 | G | A |
| PS24 | 913 | 3159 | C | T |
| PS27 | 1171 | 3417 | A | G |

Table 12 provides the SPR1 haplotypes that showed the most significant associations to PASI using a dominant genetic copy number model. In particular, the “Polymorphic Sites of SPR1 Gene” set forth in the columns of Table 12 correspond with the polymorphic sites of the SPR1 gene that are identified in Table 11. Each row of Table 12 represents a haplotype marker. In addition, “Unadjusted P-Value” and “O.R.” correspond to the raw p-value and odds ratio, respectively, for each haplotype marker within the SPR1 gene. The asterisks in Table 12 denote that the alleles at these sites are not determining and may be either allele, i.e., either the Reference Allele or the Variant Allele, as identified in Table 11. The “Lower CI of O.R.” and “Upper CI of O.R.” represent 95% confidence limits of the odds ratio.

TABLE 12
Table of SPR1 haplotypes showing association to PASI using a dominant genetic copy number model.

| PS2 | PS6 | PS7 | PS12 | PS16 | PS20 | PS21 | PS22 | PS24 | PS27 | Unadjusted | O.R. | Lower CI | Upper CI |
|---|---|---|---|---|---|---|---|---|---|---|---|---|---|
| −1119 | −845 | −455 | −384 | −228 | 161 | 627 | 739 | 913 | 1171 | p | (1 or 2 vs 0) | of O.R. | of O.R. |
| * | G | G | * | * | * | * | * | * | A | 0.0002 | 7.1 | 2.5 | 20.0 |
| * | G | G | * | A | * | * | * | * | A | 0.0003 | 6.1 | 2.3 | 16.4 |
| G | G | G | * | * | * | * | * | * | A | 0.0005 | 6.1 | 2.2 | 16.9 |
| * | G | * | * | A | * | * | G | * | A | 0.0006 | 13.6 | 3.1 | 60.4 |
| * | G | * | * | A | * | * | * | * | A | 0.0010 | 13.0 | 2.8 | 59.4 |
| * | G | G | * | * | * | * | * | C | * | 0.0014 | 5.6 | 1.9 | 16.0 |
| * | G | * | * | * | * | * | G | * | A | 0.0015 | 13.4 | 2.7 | 67.0 |
| G | G | * | * | A | * | * | * | * | A | 0.0016 | 9.7 | 2.4 | 39.6 |
| * | G | G | * | A | * | * | * | C | * | 0.0020 | 4.8 | 1.8 | 13.1 |
| * | G | G | * | * | T | * | * | * | A | 0.0020 | 4.6 | 1.7 | 12.0 |
| * | * | G | C | * | T | A | * | * | * | 0.0024 | 4.5 | 1.7 | 11.7 |
| * | G | * | * | * | * | * | * | * | A | 0.0025 | 12.6 | 2.4 | 65.1 |
| G | G | * | * | * | * | * | G | * | A | 0.0025 | 9.5 | 2.2 | 40.8 |
| * | G | G | * | * | * | A | * | * | A | 0.0030 | 4.4 | 1.6 | 11.6 |
| * | G | * | * | A | * | * | G | C | * | 0.0031 | 12.1 | 2.3 | 63.3 |
| * | * | G | * | * | T | A | * | C | * | 0.0034 | 4.3 | 1.6 | 11.2 |
| * | G | G | C | * | T | * | * | * | * | 0.0034 | 4.2 | 1.6 | 11.0 |
| G | G | G | * | * | * | * | * | C | * | 0.0035 | 4.7 | 1.7 | 13.4 |
| * | * | * | * | A | * | * | G | * | A | 0.0037 | 19.2 | 2.6 | 140.3 |
| G | G | * | * | * | * | * | * | * | A | 0.0042 | 8.9 | 2.0 | 39.9 |
| * | * | G | * | * | * | A | * | * | A | 0.0046 | 4.7 | 1.6 | 13.8 |
| * | G | * | * | A | * | * | * | C | * | 0.0048 | 11.5 | 2.1 | 62.5 |
| * | G | G | * | * | T | * | * | C | * | 0.0050 | 4.0 | 1.5 | 10.5 |

B. Haplotype Marker 3 in the SPR1 Gene

A three-SNP haplotype marker (−845,−455,1171/GGA), referred to herein as “haplotype marker 3,” was selected for detailed analysis. Haplotype marker 3 has a dominant pattern in that subjects with 0 copies are more likely to be non-responders. A summary of haplotype marker 3 is provided in Table 13.

TABLE 13
Summary of the Association Results of Haplotype Marker 3

| | Haplotype Marker 3 |
|---|---|
| | (−845, −455, 1171/GGA) |
| Subject Count (# Copy) | 28(0) 117(1, 2) |
| OR (1, 2 vs 0) | 7.1 |
| 95% Confidence Interval | 2.5, 20 |
| Unadjusted p-value | 0.0002 |
| Adjusted p-value | 0.028 |
| Copy number of haplotype marker | 0 copy |
| associated with non-response | |
| Frequency of 1 or 2 copies of the haplotype | 80.7% |
| marker in Alefacept Treated Cohort | |
| Percent of non-responders with 0 copy | 46.2% |
| Percent of strong respbnders with 0 copy | 13.4% |

C. Association Analysis of the Haplotype Marker 3 with Drug Response

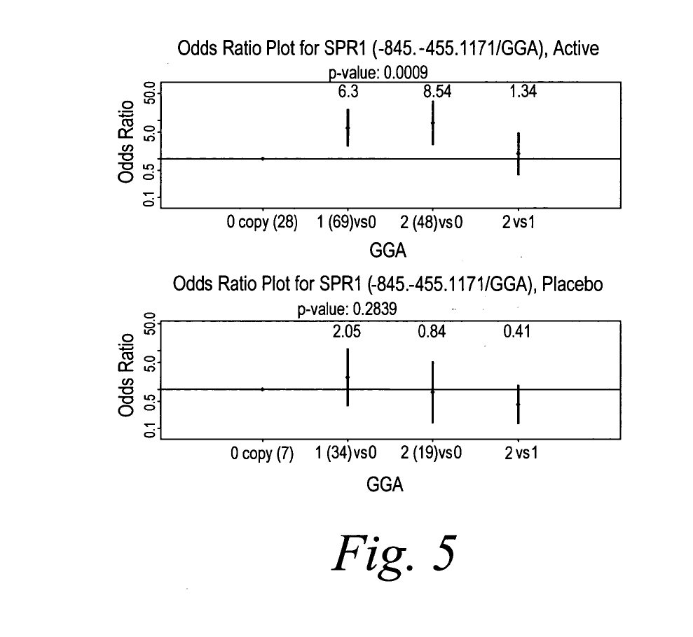

FIG. 5 depicts the results of the association analysis of haplotype marker 3 with drug response as an OR plot. The selected haplotype has a raw p-value of 0.0009 when 0, 1 or 2 copies of the marker were tested against response in the Alefacept treated patient group. The copy number of the marker has no effect on placebo response and the p-value for response to placebo is non-significant (p=0.2839) (FIG. 5, lower panel). The association remained statistically significant (permutation adjusted p=0.028) after correcting the raw p value for multiple comparisons using a permutation test.

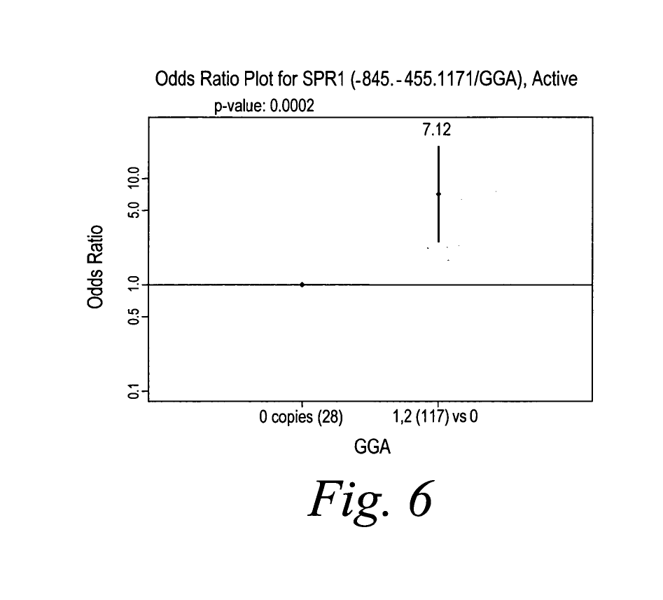

Subjects with 1 or 2 copies of the haplotype marker were collapsed into one group due to a similar likelihood of response as seen in FIG. 5. This represents a dominant model of inheritance where subjects with 1 or 2 copies are 7 times more likely to respond to Alefacept compared to those with 0 copies of the haplotype marker (FIG. 6).

The marker distribution between strong and non-responders to Alefacept is summarized in Table 14. This SPR1 marker (0 copies of GGA) identifies 46% of the non-responders and 13% of the strong responders with an OR of 7.1. If these test characteristics are applied to the distribution of response seen in the Alefacept trials (23% non-responders and 55% strong responders), SPR1 haplotype marker 3 would be predicted to have a PPV of 80% and NPV of 61% for non-response to Alefacept.

TABLE 14
Distribution of Haplotype Marker 3

| | | Strong |
|---|---|---|
| Copy Number | Non-Responder | Responder |
| 0 | 14 | 103 |
| 1 or 2 | 12 | 16 |
| Total no. of Subjects | 26 | 119 |

### Example IV

### Identification and Analysis of the Haplotype Marker 4 in the TCF19 Gene

This Example describes the identification and analysis of haplotypes in TCF19 which are strongly associated with response to Alefacept. Haplotype marker 4 was analyzed in greatest detail.

A. Polymorphic Sites Identified in the TCF19 Gene

Table 15 depicts the polymorphic sites identified in the TCF19 gene. In particular, Table 15 provides a polymorphic site number, the ATG offset of the first position of the SNP, the nucleotide position of the first position of the SNP within SEQ ID NO:4, the allele present at the ATG offset and nucleotide position, and the allele that is substituted in place of the reference allele

TABLE 15
Polymorphic Sites Identified in the TCF19 Gene

| Polymorphic | ATG | Nucleotide | Reference | Variant |
|---|---|---|---|---|
| Site Number | Offset | Position | Allele | Allele |
| PS3 | −303 | 1568 | T | C |
| PS5 | −210 | 1661 | C | T |
| PS6 | 316 | 2186 | T | C |
| PS7 | 2059 | 3929 | C | T |
| PS9 | 2365 | 4235 | G | A |
| PS11 | 2456 | 4326 | C | T |
| PS13 | 3340 | 5210 | A | G |

Tables 16A and 16B provide the TCF19 haplotypes that showed the most significant associations to PASI using a dominant and recessive genetic copy number model, respectively. In particular, the “Polymorphic Sites of TCF19 Gene” set forth in the columns of Tables 16A and 16B correspond with the polymorphic sites of the TCF19 gene that are identified in Table 15. Each row of Tables 16A and 16B represents a haplotype marker. In addition, “Unadjusted P-Value” and “O.R.” correspond to the raw p-value and odds ratio data, respectively, for each haplotype marker within the TCF19 gene. The asterisks in Table 16 denote that the alleles at these sites are not determining and may be either allele, i.e., either the Reference Allele or the Variant Allele, as identified in Table 15. The “Lower CI of O.R.” and “Upper CI of O.R.” represent 95% confidence limits of the odds ratio.

TABLE 16A
Table of TCF19 haplotypes showing association to
PASI using a dominant genetic copy number model.

| PS3 | PS5 | PS6 | PS7 | PS9 | PS11 | PS13 | Unadjusted | O.R. | Lower CI | Upper CI |
|---|---|---|---|---|---|---|---|---|---|---|
| −303 | −210 | 316 | 2059 | 2365 | 2456 | 3340 | P-Value | (1 or 2 vs 0) | of O.R. | of O.R. |
| * | * | * | * | G | * | G | 0.0015 | 0.16 | 0.05 | 0.49 |
| * | C | * | * | * | * | G | 0.0018 | 0.10 | 0.02 | 0.42 |
| * | * | * | * | * | * | G | 0.0038 | 0.12 | 0.03 | 0.50 |
| * | * | * | * | G | C | G | 0.0052 | 0.22 | 0.08 | 0.64 |
| C | C | * | * | * | * | * | 0.0080 | 0.21 | 0.06 | 0.66 |
| * | * | * | C | * | * | A | 0.0097 | 4.35 | 1.43 | 13.24 |

TABLE 16B
Table of TCF19 haplotypes showing association to
PASI using a recessive genetic copy number model.

| PS3 | PS5 | PS6 | PS7 | PS9 | PS11 | PS13 | Unadjusted | O.R. | Lower CI | Upper CI |
|---|---|---|---|---|---|---|---|---|---|---|
| −303 | −210 | 316 | 2059 | 2365 | 2456 | 3340 | P-Value | (2 vs 0 or 1) | of O.R. | of O.R. |
| * | C | * | * | * | C | G | 0.0037 | 0.13 | 0.03 | 0.52 |
| * | * | * | * | * | * | A | 0.0038 | 8.53 | 2.00 | 36.40 |
| * | C | C | * | * | C | * | 0.0051 | 0.21 | 0.07 | 0.62 |
| * | * | * | * | * | C | G | 0.0067 | 0.17 | 0.05 | 0.61 |

B. Haplotype Marker 4 in the TCF19 Gene

The association analysis of the TCF19 gene markers and PASI scores identified a haplotype marker of TCF19 (2365,3340/GG), referred to herein as “haplotype marker 4,” with a statistically significant association to response to Alefacept. A summary of haplotype marker 4 is provided in Table 17.

TABLE 17
Summary of the Association Results of Haplotype Marker 4

| | Haplotype Marker 4 |
|---|---|
| | 2365, 3340/GG |
| Copy Category | 0 vs. 1, 2 |
| Subject Count (# Copy) | 51 (0), 43 (1, 2) |
| OR (0 vs. 1, 2) | 6.3 |
| 95% Confidence Interval for OR | 2.0, 20 |
| Unadjusted p-value | 0.0015 |
| Permutation adjusted p-value | 0.01 |
| Copy number of the haplotype marker | 1, 2 copies |
| associated with no response | |
| Frequency of 1 or 2 copies of the haplotype | 38% |
| marker in Alefacept-treated Cohort | |
| Percent of non-responders with 1 or 2 copies | 73.1% |
| Percent of strong responders with 1 or 2 copies | 35.3% |

C. Association Analysis of Haplotype Marker 4 with Drug Response

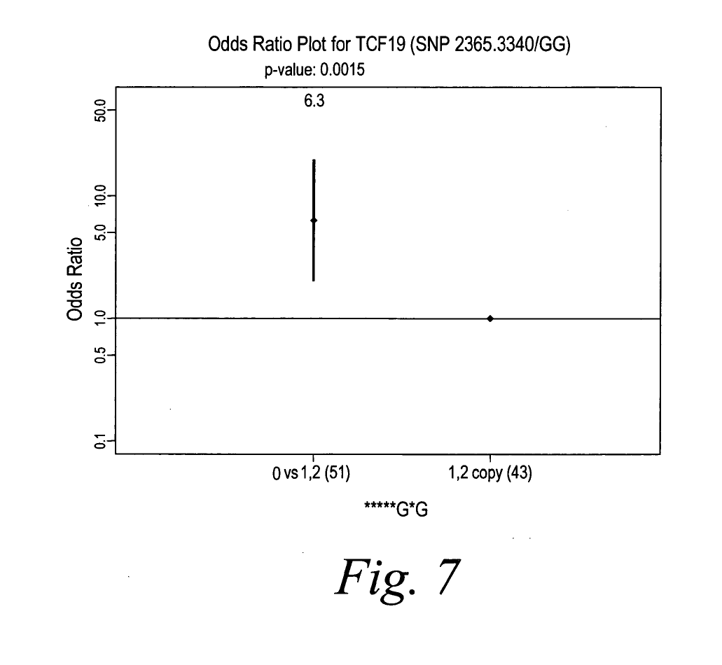

The results of the association analysis of haplotype marker 4 for Alefacept response are also depicted in an OR plot for the dominant model in FIG. 7. It indicates that people with 0 copies of this marker have 6.3 times higher odds of responding to Alefacept treatment than those with 1 or 2 copies of the marker. Haplotype marker 4 has a raw p-value of 0.0015 and a permutation adjusted p-value of 0.01. A summary of the haplotype marker 4 distribution is provided in Table 18.

TABLE 18
Distribution of Haplotype Marker 4

| Copy Number | Non-Responder | Strong Responder |
|---|---|---|
| 0 | 7 | 44 |
| 1 or 2 | 19 | 24 |
| Total no. of Subjects | 26 | 68 |

Haplotype marker 4 correctly identifies non-responders 71% of the time. If the test characteristics are applied to the distribution of response seen in the Alefacept trials (23% non-responders and 55% strong responders), this haplotype marker would be predicted to have a PPV of 86% and NPV of 53% of non-response to Alefacept.

### Example V

### Multigene Analysis

This Example describes the multigene analysis of haplotype markers in the HCR, SPR1, CD8B1 and the TCF19 genes with significant association to response to Alefacept.

The haplotype markers with significant association to response to Alefacept in HCR, SPR1, CD8B1 and the TCF19 genes are summarized in Table 19, and were considered for inclusion in two-gene models.

TABLE 19
Single gene haplotype markers included in multi-gene analyses

| | | No. Copies for | | |
|---|---|---|---|---|
| Haplotype | Genetic | Nonresponder | Unadjusted | Permutation |
| Marker | Model | Group | p-value | p-value |
| Haplotype | Recessive | 2 | 0.0003 | 0.031 |
| Marker 2 | | | | |
| Haplotype | Recessive | 2 | 0.0004 | 0.033 |
| Marker 5 | | | | |
| Haplotype | Dominant | 0 | 0.0002 | 0.028 |
| Marker 3 | | | | |
| Haplotype | Dominant | 1 or 2 | 0.0009 | 0.021 |
| Marker 1 | | | | |
| Haplotype | Dominant | 1 or 2 | 0.0015 | 0.01 |
| Marker 4 | | | | |

Since HCR and SPR1 are highly linked on the PSORS1 locus, the markers from each gene tend to be found in the same patients. Hence, this pair of genes was not analyzed in a 2-gene model.

In the two-gene modeling, combinations of copy numbers and haplotype marker for each gene (already collapsed to reflect the recessive or dominant model providing the best single gene statistical results) were evaluated. Logistic regressions were run, initially considering all four possible combinations of copy numbers for the pair of haplotype markers. Subsequently the four copy number groups were collapsed to two (either the worst group vs. the other three or the best group vs. the other three). Of the models run, none had a significant interaction p-value. However, in all the models, the 2-degree of freedom test for the addition of the main effect of the second marker and an interaction effect with the first marker were significant, regardless of the order in which the markers were added. Table 20 gives p-values for main effects, the interaction, and the 2 degrees of freedom test.

TABLE 20
Summary of Interaction Models from Multigene Analysis

| | | M1 Main | M2 Main | | | |
|---|---|---|---|---|---|---|
| | | Effect | Effect | Interaction | 2 df p-value | 2 df p-value |
| M1 | M2 | p-value | p-value | p-value | (M1 then M2) | (M2 then M1) |
| HCR-CTG | CD8B1-CTG | 0.0009 | 0.0022 | 0.8409 | 0.0062 | 0.003 |
| HCR-GGC | CD8B1-CTG | 0.0016 | 0.0028 | 0.5677 | 0.0097 | 0.0047 |
| SPR1-GGA | CD8B1-CTG | 0.0007 | 0.0102 | 0.6137 | 0.0101 | 0.0032 |
| TCF19-GG | CD8B1-CTG | 0.0027 | 0.0214 | 0.2958 | 0.0146 | 0.0035 |
| TCF19-GG | HCR-CTG | 0.0124 | 0.0043 | 0.3184 | 0.0128 | 0.0099 |
| TCF19-GG | SPR1-GGA | 0.1308 | 0.0032 | 0.1923 | 0.0074 | 0.0128 |

The marker distribution between strong responders and non-responders is summarized in the tables below.

As indicated in Table 21, the CD8B1/HCR-CTG multi-gene marker, composed of 1 or 2 copies of haplotype marker 1 and 2 copies of haplotype marker 2, correctly identifies 38% of the non-responders and falsely assigns only 5% of the strong responders as non-responders with an OR of 12. Thus, subjects who test negative for this multi-gene marker are 12 times more likely to respond to Alefacept treatment than subjects who test positive. If these test characteristics are applied to the distribution of response seen in the Alefacept trials (23% non-responders and 55% strong responders), this marker would be predicted to have a positive predictive value (PPV) of 78.8% and a negative predictive value (NPV) of 75% for non-response to Alefacept.

TABLE 21
Distribution of CD8B1/HCR-CTG multigene marker

| Copy numbers | | |
|---|---|---|
| (CD8B1/HCR) | Non-Responder | Strong Responder |
| all other combinations | 16 | 113 |
| 1 or 2/2 | 10 | 6 |
| Total no. of Subjects | 26 | 119 |

As indicated in Table 22, the CD8B1/SPR1 multi-gene marker, made up of 1 or 2 copies of haplotype marker 1 and 0 copy of haplotype marker 3, correctly identifies 31% of the non-responders and falsely assigns only 3% of the strong responders as non-responders with an OR of 11. Thus, subjects who test negative for this multi-gene marker are 11 times more likely to respond to Alefacept treatment than those who test positive. If these test characteristics are applied to the distribution of response seen in the Alefacept trials (23% non-responders and 55% strong responders), this marker would be predicted to have a PPV of 76.8% and a NPV of 77.8% for non-response to Alefacept.

TABLE 22
Distribution of CD8B1/SPR1 multigene marker

| Copy numbers | | |
|---|---|---|
| (CD8B1/SPR1) | Non-Responder | Strong Responder |
| all other combinations | 18 | 115 |
| 1 or 2/0 | 8 | 4 |
| Total no. of Subjects | 26 | 119 |

As indicated in Table 23, the TCF19/CD8B1 multi-gene marker, composed of 1 or 2 copies of haplotype marker 4 and 1 or 2 copies of haplotype marker 1, correctly identifies 91% of the responders and falsely identifies 50% of the non-responders as strong responders with an OR of 13.1. Thus, subjects who test positive for this multi-gene marker are 13 times more likely to respond to Alefacept treatment than those who test negative. If these test characteristics are applied to the distribution of response seen in the Alefacept trials (23% non-responders and 55% strong responders), this marker would be predicted to have a PPV of 80.6% and a NPV of 70.6% for non-response to Alefacept.

TABLE 23
Distribution of TCF19/CD8B1 multigene marker

| Copy numbers | | |
|---|---|---|
| (TCF19/CD8B1) | Non-Responder | Strong Responder |
| all other combinations | 13 | 6 |
| 1 or 2/1 or 2 | 13 | 62 |
| Total no. of Subjects | 26 | 68 |

As indicated in Table 24, the TCF19/HCR multi gene marker composed of 1 or 2 copies of haplotype marker 4 and 2 copies of haplotype marker 2, correctly identifies 90% of the responders and falsely identifies 58% of the non-responders as strong responders with an OR of 6.9. Thus, subjects who test positive for this marker are almost 7 times more likely to respond to Alefacept treatment than those who test negative. If these test characteristics are applied to the distribution of response seen in the Alefacept trials (23% non-responders and 55% strong responders), this marker would be predicted to have a PPV of 79.4% and a NPV of 62.5% for non-response to Alefacept.

TABLE 24
Distribution of TCF19/HCR multigene marker

| Copy numbers | | |
|---|---|---|
| (TCF19/HCR) | Non-Responder | Strong Responder |
| all other combinations | 11 | 7 |
| 1 or 2/2 | 15 | 61 |
| Total no. of Subjects | 26 | 68 |

As indicated in Table 25, the TCF19/SPR1 multi-gene marker composed of 1 or 2 copies of haplotype marker 4 and 0 copy of haplotype marker 3, correctly identifies 91% of the responders and falsely identifies 65% of the non-responders as strong responders with an OR of 7.5. Thus, subjects who test positive for this multi-gene marker are almost eight times more likely to respond to Alefacept treatment than those who test negative. If these test characteristics are applied to the distribution of response seen in the Alefacept trials (23% non-responders and 55% strong responders), this marker would be predicted to have a PPV of 76.9% and a NPV of 61.5% for non-response to Alefacept.

TABLE 25
Distribution of TCF19/SPR1 multigene marker

| Copy numbers | | |
|---|---|---|
| (TCF19/SPR1) | Non-Responder | Strong Responder |
| all other combinations | 9 | 6 |
| 1 or 2/0 | 17 | 62 |
| Total no. of Subjects | 26 | 68 |

Table 26 displays a simulation of the effects of using these genetic markers as a screening test for Alefacept therapy. In a sample of 100 patients (reflecting the response rates seen in trials of Alefacept), there would be 23 non-responders, 22 partial responders, and 55 strong responders. For each single- or multi-gene marker, the “In” group is composed of patients who would be treated based on the test results. The “Out” group is composed of those who would not be treated. Although partial responders were not included in the statistical analyses, their haplotypes are known, and hence can be included in this table. For example, using the CD8B1/SPR1 marker, 91 patients would be treated, for whom the distribution of response would be 17.6%, 24.2% and 58.2% non-, partial and strong responders, respectively; 9 patients would be denied treatment, of whom 77.8%, 0% and 22.2% would be non-, partial and strong responders.

TABLE 26
Simulated use of Haplotype Markers to Screen for treatment with Alefacept

| | Number of Patients | | Distribution of Responders in Treated and Untreated Patients | | | | | |
|---|---|---|---|---|---|---|---|---|
| | (N = 100) | | Non | | Partial | | Strong | |
| | IN | OUT | IN(%) | OUT(%) | IN(%) | OUT(%) | IN(%) | OUT(%) |
| Without test | 100 | 0 | 23% | | 22% | | 55% | |
| CD8B1 | 64 | 36 | 14.1% | 38.9% | 23.4% | 19.4% | 62.5% | 41.7% |
| HCR | 68 | 32 | 14.7% | 40.6% | 22.1% | 21.9% | 63.2% | 37.5% |
| SPR1 | 77 | 23 | 15.6% | 47.8% | 22.1% | 21.7% | 62.3% | 30.4% |
| CD8B1/HCR | 87 | 13 | 16.1% | 69.2% | 24.1% | 7.7% | 59.8% | 23.1% |
| CD8B1/SPR1 | 91 | 9 | 17.6% | 77.8% | 24.2% | 0.0% | 58.2% | 22.2% |
| TCF19* | 42 | 36 | 14.3% | 47.2% | | | 85.7% | 52.8% |
| TCF19/CD8B1* | 62 | 17 | 19.4% | 70.6% | | | 80.6% | 29.4% |
| TCF19/HCR* | 63 | 16 | 20.6% | 62.5% | | | 79.4% | 37.5% |
| TCF19/SPR1* | 65 | 13 | 23.1% | 61.5% | | | 76.9% | 38.5% |

*Excluding partial-responders in the analysis.

What is claimed:

1. A method of determining a subject's responsiveness to treatment with a T-cell depleting agent, comprising determining the nucleotide present at one or more polymorphic sites within a T cell activation or inhibition haplotype in a sample derived from said subject, thereby determining a subject's responsiveness to treatment with the T-cell depleting agent based on the nucleotide present in said subject at one or more polymorphic sites in said T cell activation or inhibition haplotype.

2. The method of claim 1, wherein the T-cell depleting agent is Alefacept.

3. The method of claim 1, wherein said T cell activation or inhibition haplotype is a haplotype in a gene selected from the group consisting of CD8B1, HCR, SPR1, and TCF19.

4. The method of claim 1, wherein said T cell activation or inhibition haplotype is a haplotype in the CD8B1 gene and wherein the p-value for the association between the haplotype and responsiveness to treatment is less than or equal to about 0.005.

5. The method of claim 1, wherein said T cell activation or inhibition haplotype is a haplotype in the SPR1 gene and wherein the p-value for the association between the haplotype and responsiveness to treatment is less than or equal to about 0.005.

6. The method of claim 1, wherein said T cell activation or inhibition haplotype is a haplotype in the TCF19 gene and wherein the p-value for the association between the haplotype and responsiveness to treatment is less than or equal to about 0.010.

7. The method of claim 1, wherein said T cell activation or inhibition haplotype is a haplotype in the HCR gene and wherein the p-value for the association between the haplotype and responsiveness to treatment is less than or equal to about 0.007.

8. The method of claim 1, wherein the T cell activation or inhibition haplotype is haplotype marker 1 and the method further comprises determining, in a sample derived from said subject, the nucleotide present at one or more polymorphic sites within a T cell activation or inhibition haplotype selected from haplotype marker 2, haplotype marker 3, haplotype marker 4 or haplotype marker 5.

9. The method of claim 1, further comprising determining the copy number of the T cell activation or inhibition haplotype using the nucleotide present in said subject at one or more polymorphic sites in said T cell activation or inhibition haplotype.

10. A kit comprising an oligonucleotide selected from the group consisting of one or more oligonucleotides suitable for genotyping an SNP in a T cell activation or inhibition haplotype in the CD8B1, HCR, SPR1, and TCF19 genes, whereby the copy number of the T cell activation or inhibition haplotype provides a statistically significant correlation with whether a group of subjects suffering from a T cell associated disease will respond or not respond to a T cell depleting agent.

11. The kit of claim 10, wherein said T cell activation or inhibition haplotype is a haplotype in the CD8B1 gene and wherein the p-value for the association between the haplotype and responsiveness to treatment is less than or equal to about 0.005.

12. The kit of claim 10, wherein said T cell activation or inhibition haplotype is a haplotype in the HCR gene and wherein the p-value for the association between the haplotype and responsiveness to treatment is less than or equal to about 0.007.

13. The kit of claim 10, wherein said T cell activation or inhibition haplotype is a haplotype in the SPR1 gene and wherein the p-value for the association between the haplotype and responsiveness to treatment is less than or equal to about 0.005.

14. The kit of claim 10, wherein said T cell activation or inhibition haplotype is a haplotype in the TCF19 gene and wherein the p-value for the association between the haplotype and responsiveness to treatment is less than or equal to about 0.010.

15. The kit set forth in claim 10, wherein the T cell depleting agent is Alefacept.

16. The kit set forth in claim 10, wherein the T cell associated disease is psoriasis.

17. A kit for detecting the presence of a T cell activation or inhibition haplotype correlated with response or nonresponse to a T-cell depleting agent, the kit comprising a set of oligonucleotides designed for genotyping the polymorphic sites within the T cell activation or inhibition haplotype, wherein the T cell activation or inhibition haplotype is a haplotype in a gene selected from the group consisting of CD8B1, HCR, SPR1, and TCF19.

18. The kit of claim 17, wherein said haplotype is selected from the group consisting of:
(a) the CD8B1 haplotypes shown in Tables 3A and 3B;
(b) the HCR haplotypes shown in Tables 7A and 7B;
(c) the SPR1 haplotypes shown in Table 12;
(d) the TCF19 haplotypes shown in Tables 16A and B;
(e) a linked haplotype to any one of:
(i)the CD8B1 haplotypes shown in Tables 3A and 3B,
(ii) the HCR haplotypes shown in Tables 7A and 7B,
(iii) the SPR1 haplotypes shown in Table 12, or
(iv) the TCF19 haplotypes shown in Tables 16A and B; and
(f) a substitute haplotype for any one of:
(i) the CD8B1 haplotypes shown in Tables 3A and 3B,
(ii) the HCR haplotypes shown in Tables 7A and 7B,
(iii) the SPR1 haplotypes shown in Table 12, or
(iv) the TCF19 haplotypes shown in Tables 16A and B.

19. The kit of claim 17, wherein said haplotype is:
(a) a haplotype marker selected from the group consisting of haplotype marker 1, haplotype marker 2, haplotype marker 3, and haplotype marker 4 and haplotype marker 5;
(b) a linked haplotype to haplotype marker 1, haplotype marker 2, haplotype marker 3, haplotype marker 4 or haplotype marker 5; or
(c) a substitute haplotype for haplotype marker 1, haplotype marker 2, haplotype marker 3, haplotype marker 4 or haplotype marker 5.

20. The kit set forth in claim 19, wherein linkage disequilibrium between the linked haplotype marker and the haplotype marker has a Δ² selected from the group consisting of at least 0.75, at least 0.80, at least 0.85, at least 0.90, at least 0.95, and 1.0.

21. The kit of claim 20, wherein Δ² is at least 0.95.

22. The kit set forth in claim 17, wherein the T cell depleting agent is Alefacept.

23. A kit comprising an oligonucleotide selected from the group consisting of one or more oligonucleotides suitable for genotyping an SNP in the CD8B1, HCR, SPR1, and TCF19 genes for diagnosing the response of a subject suffering from a disease to a treatment regime.

24. The kit of claim 23, wherein the SNP is selected from the polymorphisms at: positions −685, −255, 25, 8632, 15080, 19501, 28589, 28663 and 28739 in the CD8B1 gene, positions 2173, 2175, 2360, 5782, 5787, 6174, 6666, 8277, 8440, 8476, 11565, 11941, 12152, 13553, 13892, 14287 in the HCR gene, positions −119, −845, −455, −384, −228, 161, 627, 739, 913 and 1171 in the SPR1 gene, and −303, −210, 316, 2059, 2365, 2456 and 3340 in the TCF19 gene.

25. The kit of claim 23, further comprising instructions of use.

26. The kit of claim 23, wherein the oligonucleotide is capable of detectably hybridizing to the SNP.

27. A single-stranded oligonucleotide suitable for genotyping an SNP in a T cell activation or inhibition haplotype in the CD8B1, HCR, SPR1, or TCF19 genes.

28. The single-stranded oligonucleotides of claim 27, wherein the SNP is selected from the polymorphisms at: positions −685, −255, 25, 8632, 15080, 19501, 28589, 28663 and 28739 in the CD8B1 gene, positions 2173, 2175, 2360, 5782, 5787, 6174, 6666, 8277, 8440, 8476, 11565, 11941, 12152, 13553, 13892, 14287 in the HCR gene, positions −119, −845, −455, −384, −228, 161, 627, 739, 913 and 11711 in the SPR1 gene, and −303, −210, 316, 2059, 2365, 2456 and 3340 in the TCF19 gene.

29. A method of determining a subject's responsiveness to treatment with a T-cell depleting agent, comprising analyzing a sample derived from said subject to determine the subject's copy number for a T cell activation or inhibition haplotype.

30. The method of claim 29, wherein the T-cell depleting agent is Alefacept.

31. The method of claim 29, wherein said T cell activation or inhibition haplotype is a haplotype in a gene selected from the group consisting of CD8B1, HCR, SPR1, and TCF19.

32. The method of claim 31, wherein said haplotype is selected from the group consisting of:
(a) the CD8B1 haplotypes shown in Tables 3A and 3B;
(b) the HCR haplotypes shown in Tables 7A and 7B;
(c) the SPR1 haplotypes shown in Table 12;
(d) the TCF19 haplotypes shown in Tables 16A and B;
(e) a linked haplotype to any one of:
(i) the CD8B1 haplotypes shown in Tables 3A and 3B,
(ii) the HCR haplotypes shown in Tables 7A and 7B,
(iii) the SPR1 haplotypes shown in Table 12, or
(iv) the TCF19 haplotypes shown in Tables 16A and B; and
(f) a substitute haplotype for any one of:
(i) the CD8B1 haplotypes shown in Tables 3A and 3B,
(ii) the HCR haplotypes shown in Tables 7A and 7B,
(iii) the SPR1 haplotypes shown in Table 12, or
(iv) the TCF19 haplotypes shown in Tables 16A and B.
